SEQUENCE LISTING (77 sequences)

SEQ ID NO: 1 - protein, 25 aa, Artificial Sequence
  Synthetic sequence - KRAS G12V Immunogenic Peptide
MTEYKLVVVGAVGVGKSALTIQLIQ

SEQ ID NO: 2 - protein, 16 aa, Artificial Sequence
  Synthetic sequence - CDR3 alpha
CAVGRSNSGGYQKVTF

SEQ ID NO: 3 - protein, 15 aa, Artificial Sequence
  Synthetic sequence - CDR3 beta
CAWSALAGARDTQYF

SEQ ID NO: 4 - DNA, 48 bp, Artificial Sequence
  Synthetic sequence - CDR3 alpha native nucleic acid
tgtgctgtgggccgatcgaattctgggggttaccagaaagttaccttt

SEQ ID NO: 5 - DNA, 48 bp, Artificial Sequence
  Synthetic sequence - CDR3 alpha codon optimized nucleic acid
tgcgccgtgggcagatctaatagcggcggctaccagaaagtgaccttt

SEQ ID NO: 6 - protein, 310 aa, Artificial Sequence
  Synthetic sequence - Vbeta amino acid
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSALAGARDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - DNA, 45 bp, Artificial Sequence
  Synthetic sequence - CDR3 beta native nucleic acid
tgtgcctggagtgcgttggcgggagctagagatacgcagtatttt

SEQ ID NO: 8 - DNA, 45 bp, Artificial Sequence
  Synthetic sequence - CDR3 beta codon optimized nucleic acid
tgtgcttggagtgctctggcaggcgccagagatacccagtatttt

SEQ ID NO: 9 - protein, 276 aa, Artificial Sequence
  Synthetic sequence - Valpha amino acid
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGRSNSGGYQKVTFGIGTKLQVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - DNA, 1827 bp, Artificial Sequence
  Synthetic sequence - Entire TCR transgene (Nucleic acid codon optimized)
atgctttgttctttgctggctctgctgctgggcaccttctttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagtgctctggcaggcgccagagatacccagtattttggccctggcaccagactgaccgtgctggaagatctgaagaacgtgttcccacctgaggtggccgtgttcgagccttctgaggccgagatcagccacacacagaaagccacactcgtgtgtctggccaccggcttttaccccgatcacgtggaactgtcttggtgggtcaacggcaaagaggtgcacagcggcgtctgcacagatccccagcctctgaaagaacagcccgctctgaacgacagccggtactgcctgtctagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggaaaagccactctgtacgccgtgctggtgtctgccctggtgctgatggccatggtcaagcggaaggatagcagaggcggaagcggcgccacaaacttcagcctgcttaaacaggccggcgacgtggaagagaaccccggacctatgctgctggaactgatccctctgctggggattcacttcgtgctgagaaccgctagagcccagagcgtgacccagcctgatatccacatcaccgtgtctgagggcgcctctctggaactgcggtgcaattacagctacggcgccactccttacctgttttggtacgtgcagagcccaggccagggactgcaactcctgctgaagtactttagcggcgacaccctggtgcagggcatcaagggattcgaggccgagttcaagagaagccagtccagcttcaacctgcggaagccttccgtgcattggagtgatgccgccgagtacttttgcgccgtgggcagatctaatagcggcggctaccagaaagtgacctttggcatcggcaccaagctgcaagtgatccccaacattcagaaccccgatcctgcagtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 11 - protein, 608 aa, Artificial Sequence
  Synthetic sequence - Entire TCR transgene AA (beta, P2A, alpha)
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSALAGARDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGRSNSGGYQKVTFGIGTKLQVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - protein, 16 aa, Artificial Sequence
  Synthetic sequence - CDR3 alpha
CAVTVVNAGNNRKLIW

SEQ ID NO: 13 - protein, 15 aa, Artificial Sequence
  Synthetic sequence - CDR3 beta
CASSLGLPGTDTQYF

SEQ ID NO: 14 - DNA, 48 bp, Artificial Sequence
  Synthetic sequence - CDR3 alpha native nucleic acid
tgtgccgtgacggtggttaatgctggcaacaaccgtaagctgatttgg

SEQ ID NO: 15 - DNA, 48 bp, Artificial Sequence
  Synthetic sequence - CDR3 alpha codon optimized nucleic acid
tgcgccgtgaccgtggtcaacgccggcaacaacagaaagctgatctgg

SEQ ID NO: 16 - protein, 313 aa, Artificial Sequence
  Synthetic sequence - Vbeta amino acid
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGLPGTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - DNA, 45 bp, Artificial Sequence
  Synthetic sequence - CDR3 beta native nucleic acid
tgtgccagcagtttagggctaccagggacagatacgcagtatttt

SEQ ID NO: 18 - DNA, 45 bp, Artificial Sequence
  Synthetic sequence - CDR3 beta codon optimized nucleic acid
tgtgccagcagcctgggactgcccggcaccgatacacagtatttt

SEQ ID NO: 19 - protein, 276 aa, Artificial Sequence
  Synthetic sequence - Valpha amino acid
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVTVVNAGNNRKLIWGLGTSLAVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - DNA, 1836 bp, Artificial Sequence
  Synthetic sequence - Entire TCR transgene (Nucleic acid codon optimized)
atgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttttgtgccagcagcctgggactgcccggcaccgatacacagtattttggccctggcaccagactgaccgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggcggaagcggcgccacaaacttctcactgctgaaacaggccggcgacgtggaagagaaccctggacctatgctgctcctgctgatccctgtgctgggcatgatcttcgccctgagagatgctagagcccagtccgtgtctcagcacaaccaccatgtgatcctgagcgaagccgcctctctggaactgggctgcaattacagctacggcggcaccgtgaatctgttttggtacgtgcagtaccccggccagcatctccagctgctgctgaagtactttagcggcgaccctctggtcaagggcatcaagggattcgaggccgagttcatcaagagcaagttcagcttcaacctgcggaagcccagcgtgcagtggagcgatacagccgagtatttctgcgccgtgaccgtggtcaacgccggcaacaacagaaagctgatctggggcctgggcaccagcctggctgtgaaccccaatattcagaaccccgatcctgcagtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 21 - protein, 611 aa, Artificial Sequence
  Synthetic sequence - Entire TCR transgene AA (beta, P2A, alpha)
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGLPGTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVTVVNAGNNRKLIWGLGTSLAVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 32 aa, Artificial Sequence
  Synthetic sequence - HER2 ITD Immunogenic Peptide
SPKANKEILDEAYVMAYVMAGVGSPYVSRLLG

SEQ ID NO: 23 - protein, 12 aa, Artificial Sequence
  Synthetic sequence - CDR3 alpha
CAVSVNTDKLIF

SEQ ID NO: 24 - protein, 14 aa, Artificial Sequence
  Synthetic sequence - CDR3 beta
CSAPPLAGDETQYF

SEQ ID NO: 25 - DNA, 36 bp, Artificial Sequence
  Synthetic sequence - CDR3 alpha native nucleic acid
tgtgctgtgagtgtgaacaccgacaagctcatcttt

SEQ ID NO: 26 - DNA, 36 bp, Artificial Sequence
  Synthetic sequence - CDR3 alpha codon optimized nucleic acid
tgcgccgtgtctgtgaacaccgacaagctgatcttc

SEQ ID NO: 27 - protein, 272 aa, Artificial Sequence
  Synthetic sequence - Valpha amino acid
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVSVNTDKLIFGTGTRLQVFPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - DNA, 42 bp, Artificial Sequence
  Synthetic sequence - CDR3 beta native nucleic acid
tgcagtgctcctcccctagcgggagatgagacccagtacttc

SEQ ID NO: 29 - DNA, 42 bp, Artificial Sequence
  Synthetic sequence - CDR3 beta nucleic acid - codon optimized
tgtagcgcccctccactggccggcgacgagacacaatatttt

SEQ ID NO: 30 - protein, 308 aa, Artificial Sequence
  Synthetic sequence - Vbeta Amino Acid
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAPPLAGDETQYFGPGTRLLVLDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - DNA, 1809 bp, Artificial Sequence
  Synthetic sequence - Entire Nucleic Acid TCR Transgene
atgttgcttttgttgctgctcctcggacctggctctggactgggagctgtggtttctcagcacccctcttgggtcatctgcaagagcggcaccagcgtgaagatcgagtgcagaagcctggacttccaggccaccacaatgttctggtacagacagttccccaagcagagcctgatgctgatggccacctctaacgagggcagcaaggccacatatgagcagggcgtcgagaaggacaagttcctgatcaaccacgccagcctgacactgagcaccctgacagtgacaagcgcccatcctgaggacagcagcttctacatctgtagcgcccctccactggccggcgacgagacacaatattttggccctggcaccagactgctggtgctggacctgaagaacgtgttcccacctgaggtggccgtgttcgagccttctgaggccgagatcagccacacacagaaagccacactcgtgtgtctggccaccggcttctatcccgatcacgtcgaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcaaggcgtgctgtctgccaccatcctgtacgagatcctgctgggcaaagccactctgtacgccgtgctggtgtctgccctggtcctgatggctatggtcaagcggaaggatagcagaggcggaagcggcgccacaaacttctcactgctgaaacaggcaggcgacgtggaagagaaccccggacctatgctgctgcttctggtgcctgccttccaagtgatctttaccctcggcggcacaagagcccagagcgtgacacagctggatagccaggtgccagtgttcgaagaggcccctgtggaactgcggtgcaactacagcagcagcgtgtccgtgtacctgttttggtacgtgcagtaccccaaccagggcctgcagctgctcctgaagtatctgagcggcagcaccctggtggaatccatcaatggcttcgaggccgaattcaacaagagccagaccagcttccacctgagaaagcccagcgtgcacatcagcgataccgccgagtacttctgcgccgtgtctgtgaacaccgacaagctgatcttcggcaccggcacaaggctccaggtgttccccaacattcagaaccccgatcctgcagtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 32 - protein, 602 aa, Artificial Sequence
  Synthetic sequence - Entire TCR Amino Acid Sequence
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAPPLAGDETQYFGPGTRLLVLDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVSVNTDKLIFGTGTRLQVFPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - protein, 25 aa, Artificial Sequence
  Synthetic sequence - KRAS WT peptide
MTEYKLVVVGAGGVGKSALTIQLIQ

SEQ ID NO: 34 - protein, 28 aa, Artificial Sequence
  Synthetic sequence - HER2 WT Peptide
SPKANKEILDEAYVMAGVGSPYVSRLLG

SEQ ID NO: 35 - protein, 21 aa, Artificial Sequence
  Synthetic sequence -Thoseaasigna virus 2A (T2A) peptide
GSGEGRGSLLTCGDVEENPGP

SEQ ID NO: 36 - protein, 22 aa, Artificial Sequence
  Synthetic sequence - Porcine teschovirus-1 2A (P2A) peptide
GSGATNFSLLKQAGDVEENPGP

SEQ ID NO: 37 - protein, 23 aa, Artificial Sequence
  Synthetic sequence - Equine rhinitis A virus (ERAV) 2A (E2A)
GSGQCTNYALLKLAGDVESNPGP

SEQ ID NO: 38 - protein, 25 aa, Artificial Sequence
  Synthetic sequence - Foot-and-mouth disease virus 2A (F2A) peptide
GSGVKQTLNFDLLKLAGDVESNPGP

SEQ ID NO: 39 - protein, 10 aa, Artificial Sequence
  Synthetic sequence - mutant peptide
TERWDNLIYY

SEQ ID NO: 40 - protein, 10 aa, Artificial Sequence
  Synthetic sequence - mutant peptide
AERWDNLIYY

SEQ ID NO: 41 - DNA, 27 bp, Artificial Sequence
  Synthetic sequence - Human TCR Cbeta1 Reverse
ccacttccagggctgccttcagaaatc

SEQ ID NO: 42 - DNA, 27 bp, Artificial Sequence
  Synthetic sequence - Human TCR Cbeta2 Reverse
tgggatggttttggagctagcctctgg

SEQ ID NO: 43 - DNA, 25 bp, Artificial Sequence
  Synthetic sequence - Human TCR Calpha Reverse
cagccgcagcgtcatgagcagatta

SEQ ID NO: 44 - DNA, 20 bp, Artificial Sequence
  Synthetic sequence - JV298 primer
tcgcttctgttcgcgcgctt

SEQ ID NO: 45 - DNA, 20 bp, Artificial Sequence
  Synthetic sequence - JV300 primer
aacaggcacacgctcttgtc

SEQ ID NO: 46 - DNA, 20 bp, Artificial Sequence
  Synthetic sequence - guide rna
agagtctctcagctggtaca

SEQ ID NO: 47 - DNA, 20 bp, Artificial Sequence
  Synthetic sequence - TCR beta guide rna
ggagaatgacgagtggaccc

SEQ ID NO: 48 - protein, 6 aa, Artificial Sequence
  Synthetic sequence - KRAS G12V clone 3 CDR1alpha
YGATPY

SEQ ID NO: 49 - protein, 7 aa, Artificial Sequence
  Synthetic sequence - KRAS G12V clone 3 CDR2alpha
YFSGDTL

SEQ ID NO: 50 - protein, 20 aa, Artificial Sequence
  Synthetic sequence - KRAS G12V clone 3 TCRalpha signal peptide
MLCSLLALLLGTFFGVRSQT

SEQ ID NO: 51 - protein, 6 aa, Artificial Sequence
  Synthetic sequence - KRAS G12V clone 3 CDR1beta
GTSNPN

SEQ ID NO: 52 - protein, 5 aa, Artificial Sequence
  Synthetic sequence - KRAS G12V clone 3 CDR2beta
SVGIG

SEQ ID NO: 53 - protein, 18 aa, Artificial Sequence
  Synthetic sequence - KRAS G12V clone 3 TCRbeta signal peptide
MLLELIPLLGIHFVLRTA

SEQ ID NO: 54 - protein, 6 aa, Artificial Sequence
  Synthetic sequence - KRAS G12V clone 9 CDR2alpha
YGGTVN

SEQ ID NO: 55 - protein, 7 aa, Artificial Sequence
  Synthetic sequence - KRAS G12V clone 9 CDR2alpha
YFSGDPL

SEQ ID NO: 56 - protein, 20 aa, Artificial Sequence
  Synthetic sequence - KRAS G12V clone 9 TCRalpha signal peptide
MGSWTLCCVSLCILVAKHTD

SEQ ID NO: 57 - protein, 5 aa, Artificial Sequence
  Synthetic sequence - KRAS G12V clone 9 CDR1beta
SGHDY

SEQ ID NO: 58 - protein, 6 aa, Artificial Sequence
  Synthetic sequence - KRAS G12V clone 9 CDR2beta
FNNNVP

SEQ ID NO: 59 - protein, 21 aa, Artificial Sequence
  Synthetic sequence - KRAS G12V clone 9 TCRbeta signal peptide
MLLLLIPVLGMIFALRDARAQ

SEQ ID NO: 60 - protein, 6 aa, Artificial Sequence
  Synthetic sequence - Her2 ITD TCR CDR1alpha
SSVSVY

SEQ ID NO: 61 - protein, 7 aa, Artificial Sequence
  Synthetic sequence - Her2 ITD TCR CDR2alpha
YLSGSTL

SEQ ID NO: 62 - protein, 20 aa, Artificial Sequence
  Synthetic sequence - Her2 ITD TCRalpha signal peptide
MLLLLVPAFQVIFTLGGTRA

SEQ ID NO: 63 - protein, 6 aa, Artificial Sequence
  Synthetic sequence - Her2 ITD TCR CDR1beta
DFQATT

SEQ ID NO: 64 - protein, 7 aa, Artificial Sequence
  Synthetic sequence - Her2 ITD TCR CDR2beta
SNEGSKA

SEQ ID NO: 65 - protein, 15 aa, Artificial Sequence
  Synthetic sequence - Her2 ITD TCRbeta signal peptide
MLLLLLLLGPGSGLG

SEQ ID NO: 66 - protein, 118 aa, Artificial Sequence
  Synthetic sequence - KRAS G12V clone 3 TCR Valpha (mature)
RAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGRSNSGGYQKVTFGIGTKLQVIPN

SEQ ID NO: 67 - protein, 140 aa, Artificial Sequence
  Synthetic sequence - KRAS G12V clone 3 TCR Calpha
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - protein, 112 aa, Artificial Sequence
  Synthetic sequence - KRAS G12V clone 3 TCR Vbeta (mature)
IHQWPATLVQPVGSPLSLECTVEGTSNPNLYWYRQAAGRGLQLLFYSVGIGQISSEVPQNLSASRPQDRQFILSSKKLLLSDSGFYLCAWSALAGARDTQYFGPGTRLTVLE

SEQ ID NO: 69 - protein, 178 aa, Artificial Sequence
  Synthetic sequence - KRAS G12V clone 3 TCR Cbeta
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 70 - protein, 115 aa, Artificial Sequence
  Synthetic sequence - KRAS G12V clone 9 TCR Valpha (mature)
SVSQHNHHVILSEAASLELGCNYSYGGTVNLFWYVQYPGQHLQLLLKYFSGDPLVKGIKGFEAEFIKSKFSFNLRKPSVQWSDTAEYFCAVTVVNAGNNRKLIWGLGTSLAVNPN

SEQ ID NO: 71 - protein, 140 aa, Artificial Sequence; identical to SEQ ID NO: 67
  Synthetic sequence - KRAS G12V clone 9 TCR Calpha

SEQ ID NO: 72 - protein, 115 aa, Artificial Sequence
  Synthetic sequence - KRAS G12V clone 9 TCR Vbeta (mature)
AGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGLPGTDTQYFGPGTRLTVLE

SEQ ID NO: 73 - protein, 178 aa, Artificial Sequence; identical to SEQ ID NO: 69
  Synthetic sequence - KRAS G12V clone 9 TCR Cbeta

SEQ ID NO: 74 - protein, 112 aa, Artificial Sequence
  Synthetic sequence -  HER2 ITD TCR Valpha (mature)
QSVTQLDSQVPVFEEAPVELRCNYSSSVSVYLFWYVQYPNQGLQLLLKYLSGSTLVESINGFEAEFNKSQTSFHLRKPSVHISDTAEYFCAVSVNTDKLIFGTGTRLQVFPN

SEQ ID NO: 75 - protein, 140 aa, Artificial Sequence; identical to SEQ ID NO: 67
  Synthetic sequence -  HER2 ITD TCR Calpha

SEQ ID NO: 76 - protein, 115 aa, Artificial Sequence
  Synthetic sequence -  HER2 ITD TCR Vbeta (mature)
AVVSQHPSWVICKSGTSVKIECRSLDFQATTMFWYRQFPKQSLMLMATSNEGSKATYEQGVEKDKFLINHASLTLSTLTVTSAHPEDSSFYICSAPPLAGDETQYFGPGTRLLVL

SEQ ID NO: 77 - protein, 178 aa, Artificial Sequence; identical to SEQ ID NO: 69
  Synthetic sequence - HER2 ITD TCR Cbeta

(19) United States
(12) Patent Application Publication
(10) Pub. No.: US 2021/0340201 A1
(43) Pub. Date: Nov. 4, 2021

(54) IMMUNOTHERAPY TARGETING KRAS OR HER2 ANTIGENS

(71) Applicant: FRED HUTCHINSON CANCER RESEARCH CENTER, Seattle, WA (US)

(72) Inventors: Joshua VEATCH, Seattle, WA (US); Stanley R. RIDDELL, Sammamish, WA (US)

(21) Appl. No.: 17/270,400

(22) PCT Filed: Aug. 21, 2019
(86) PCT No.: PCT/US2019/047550

§ 371 Date: Feb. 22, 2021

Related U.S. Application Data
(60) Provisional application No. 62/721,439, filed on Aug. 22, 2018.

(51) Int. Cl. C07K 14/47; A61P 35/00; A61K 35/17; C07K 14/725

(52) U.S. Cl. CPC C07K 14/4748; A61P 35/00; A61K 38/00; C07K 14/7051; A61K 35/17

(57) ABSTRACT

Binding proteins and high affinity recombinant T cell receptors (TCRs) specific for KRAS G12V or Her2-ITD neoantigens are provided herein. Compositions and recombinant host cells encoding and/or expressing the binding proteins and/or high affinity recombinant TCRs are also provided. The compositions and recombinant host cells may be used to treat a subject having non-small cell lung cancer (NSCLC), colorectal cancer, pancreas cancer, ovarian cancer, breast cancer, biliary tract cancer, an indication wherein a KRAS G12V neoantigen is a therapeutic target, or an indication wherein a Her2-ITD neoantigen is a therapeutic target. Related vaccines, vaccine therapies, and vaccination regimens are also provided.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_472WO_SEQUENCE_LISTING.txt. The text file is 61.6 KB, was created on Aug. 21, 2019, and is being submitted electronically via EFS-Web.

### TECHNICAL FIELD

The present disclosure relates to the field of biomedicine and, specifically, to compositions and methods useful for treating diseases characterized by or associated with KRAS or HER2 antigens, such as cancers. Certain embodiments of the present disclosure relate to compositions and methods for cellular immunotherapy comprising immune cells modified to encode and/or express antigen-specific binding proteins.

### BACKGROUND

T cells can eliminate cancer cells through recognition of peptides derived from the processing of non-mutated or mutated proteins and presented bound to cell surface major histocompatibility complex (MHC) molecules. T cells specific for neoantigens encoded by mutated genes have been implicated as important mediators of antitumor immunity in patients receiving checkpoint blocking antibodies (see McGranahan N, et al. Science. 2016; 351(6280):1463-69) and adoptive T cell transfer (see Lu Y-C, et al. Clinical Cancer Research. 2014; 20(13):3401-10). Neoantigens are attractive targets for T cells because they are not subject to central and peripheral tolerance mechanisms that limit the frequency and function of T cells specific for self-antigens (see Schumacher Tenn., et al. Science. 2015; 348(6230):69-74). Indeed, the burden of somatic mutations present in non-small cell lung cancer (NSCLC) and other cancer types correlates with response to immune checkpoint inhibitors (see Rizvi N A, et al. Science. 2015; 348(6230):124-8 and Yatim N, et al. Science. 2015; 350(6258):328-34), suggesting reinvigoration of endogenous neoantigen-reactive T cells contribute to efficacy. Clinical response in patients with melanoma and cervical cancer treated with tumor infiltrating lymphocytes (TILs) has also correlated with the presence of neoantigen-reactive T cells in the TIL product (see Lu Y-C, et al. Clinical Cancer Research. 2014; 20(13):3401-10). Most neoantigens are random, patient-specific, and/or heterogeneously expressed in tumors, which limits their utility as targets for adoptive transfer with engineered T cells across multiple patients (see Schumacher Tenn., et al. Science. 2015; 348(6230):69-74), and can allow escape of tumor cells that lose immunogenic neoantigens during NSCLC progression (see Anagnostou V, et al. Cancer discovery. 2017; 7(3):264-76). In contrast, recurrent oncogenic driver mutations are expressed clonally and homogenously in cancers from many patients. Unfortunately, T cell responses to very few driver mutations have been described, perhaps as a consequence of immune selection based on a human leukocyte antigen (HLA) genotype (see Marty R, et al. Cell. 2017) or the development of irreversible T cell exhaustion that precludes their isolation using functional assays (see Philip M, et al. Nature. 2017; 545(7655):452).

Efforts to identify neoantigens recognized by T cells, including those arising from oncogenic mutations, have largely focused on epitopes presented on class I MHC to CD8⁺ T cells due to their direct cytotoxic function. A role for CD4⁺ class II MHC-restricted T cells in human antitumor immunity is increasingly appreciated, despite the absence of class II MHC on many tumors. CD4⁺ T cells can recognize tumor antigen presented by professional antigen presenting cells and support the priming and expansion of CD8⁺ T cells in lymphoid tissues and the effector function of CD8⁺ T cells and innate immune cells in the tumor microenvironment. Recent work in mouse models has suggested that CD4⁺ T cells at the site of the tumor are a critical component of immune mediated tumor rejection (see Spitzer M H, et al. Cell. 2017; 168(3):487-502. e15), and that vaccination to augment class II MHC-restricted CD4⁺ T cells to neoantigens can have potent therapeutic effects (see Kreiter S, et al. Nature. 2015; 520(7549):692-6). Furthermore, CD4⁺ T cell responses to neoantigens are common in patients with melanoma (see Linnemann C, et al. Nature medicine. 2015; 21(1):81), and a recent study in melanoma patients vaccinated with candidate neoantigen peptides intending to induce CD8⁺ T cell responses instead led to CD4⁺ T cell responses to 60% of the peptides, with evidence of antitumor activity (see Ott P A, et al. Nature. 2017; 547(7662):217). The association of peritumoral CD4⁺ T cells with improved prognosis in NSCLC (see Al-Shibli K I, et al. Clinical cancer research. 2008; 14(16):5220-7; Hiraoka K, et al. British journal of cancer. 2006; 94(2):275; and Wakabayashi O, et al. Cancer science. 2003; 94(11):1003-9) suggests that anti-tumor CD4⁺ T cell responses could have clinical relevance. Nonetheless, the role of CD4⁺ neoantigen-specific T cells in human antitumor immunity is largely unknown, and few reports have specifically examined neoantigen-specific CD4⁺ T cell responses in NSCLC.

### BRIEF DESCRIPTION OF THE DRAWINGS

The embodiments disclosed herein will become more fully apparent from the following description and appended claims, taken in conjunction with the accompanying drawings.

FIGS. 1A-1E show detection and testing of CD4+ neoantigen-reactive T cells in lung cancer patients. (A) Schema for detecting neoantigen reactive T cells. Peripheral blood mononuclear cells (PBMC) from five different lung cancer patients were stimulated with pools of peptides containing mutations (B). IFN-γ secreting cells were quantitated in stimulated cultures by ELISpot after incubation with single mutant or wild-type peptides. All experiments included two or three technical replicates. (C) Representative IFN-γ intracellular staining of CD4+ and CD8+ T cells from patient 1490 after incubation with mutant SREK peptide. (D) Representative IFN-γ intracellular staining of CD4+ and CD8+ T cells from patient 1347 incubated with ZNF292 peptide. (E) Quantitation of neoantigen-specific CD4+ or CD8+ IFN-γ+ cells as a fraction of total T cells in cultures from patients 1490 and 1347.

FIGS. 2A-2D show detection and testing of neoantigen-specific CD8 T cells from tumor infiltrating lymphocytes from patient 1490. Tumor infiltrating lymphocytes from tumor resection of patient 1490 were incubated with peptides containing mutant or wild-type sequences from PWP2, and IFN-γ secretion was measured by interferon capture (A). (B) TCR Vβ clonotype frequency of PWP2-reactive CD8⁺ TCR Vβ in non-adjacent lung tissue and in tumor following tumor infiltrating lymphocyte culture, and following IFN-γ capture of TIL product. T cell line containing PWP2-specific cells were incubated with indicated concentrations of mutant (TERWDNLIYY (SEQ ID NO:39)) or wild-type (AERWDNLIYY (SEQ ID NO:40)) peptide and IFN-γ secretion was measured by ELISA (C, D).

FIGS. 3A-3G show CD4+ T cell lines specific for mutant peptides relative to wild-type peptides. Monoclonal CD4+ T cell lines from patients 1347 and 1490 enriched for antigen specific cells by IFN-γ capture were expanded in vitro and then incubated with autologous B cells and the indicated concentration of mutant or wild-type peptide. IFN-γ secretion was measured by ELISA. (A) Reactivity of T cells from patient 1347 reactive to MP3KP peptides. (B-D) Reactivity of T cells from patient 1490 to SREK1 peptides. (E, F) Reactivity of T cells from patient 1490 to GUCY1A3 peptides. (G) Reactivity of T cells from patient 1490 to AGO2 peptides.

FIGS. 4A-4K show activity and testing of CD4+ T cells specific for KRAS G12V peptides. (A) Three CD4+ T cell clones from patient 1139 (clone #s 3, 5, and 9) were incubated in the presence of the indicated concentration of the N terminal 26 amino acids of KRAS with either V12 (mutant) or G12 (wild-type) and IFN-γ production was measured by ELISA. (B) T cell clones were incubated with KRAS G12V peptide in the presence of the indicated class II HLA-blocking antibodies. (C) T cell clones were incubated with B-LCL cell lines that were pulsed with KRAS G12V peptide or control and expressed individual class II HLA alleles shared with patient 1139 (HLA DQB1-1104/1301 DQB1 0301/0603). (D) HLA DRB1-11:04+ LCL were incubated with KRAS G12V peptide or transfected with RNA encoding wildtype or G12V KRAS sequences. (E) T-cell clones were incubated with HLADRB1*11:04+ LCLs pulsed with KRAS G12V peptide (1 μg/mL) or transfected with RNA encoding wild-type or KRAS G12V sequences, and IFNγ production was measured by ELISA. (F) CD4+ T cells from two normal donors were transduced with lentiviral vectors encoding T cell receptor (TCR) Vα and Vβ genes from T cell clones #3 and #9 and then incubated HLA-DRB1-1104+ LCL cells pulsed with KRAS G12V peptide. IFN-γ secretion was measured by ELISA. (G-K) CD4+ T cells from 2 normal donors were transduced with lentiviral vectors encoding T-cell receptor Vα and Vβ genes from T-cell clones #3 (aka TCR132) and #9 (aka TCR136) with concurrent CRISPR-mediated disruption of exon 1 of the endogenous TCRα (J), and then incubated HLA-DRB1*1104+ LCL cells pulsed with KRASG12V peptide (G, H) or B-LCL cells transfected with mutant or wild-type KRAS sequences (I), and IFNγ production was measured by ELISA (G-I, K).

FIGS. 5A-5L show CD4+ T cells specific for the Her2 exon 20 insertion (ERBB2 (Her2) internal tandem duplication (ITD); also referred to herein as Her2-ITD). (A, B) A CD4+ T cell line from patient 1238 (50,000 cells) was co-cultured with autologous B cells (100,000 cells) in the presence of the indicated concentrations of Her2-ITD (SPKANKEILDEAYVMAYVMAGVGSPYVSRLLG; SEQ ID NO:22) or the corresponding wild-type peptide (SPKANKEILDEAYVMAGVGSPYVSRLLG; SEQ ID NO:34) and IFN-γ production was measured by ELISA. (C, D) The CD4+ T cell line from patient 1238 was incubated with Her2-ITD peptide in the presence of the indicated class II MHC blocking antibodies. (E, F) The CD4+ T cell line was incubated with autologous B cells pulsed with Her2-ITD peptide or transfected with RNA encoding wild-type or Her2-ITD sequences. (G) The CD4+ T cell line was incubated with Her2-ITD peptide pulsed B-LCL cell lines expressing individual class II HLA alleles shared with patient 1238 (HLA-DQB1-1202/1502 DQB1 0301/0501). (H-J) CD4+ T cells from two normal donors were transduced with TCR sequences obtained from Her2-ITD specific T cells, incubated with B cells pulsed with Her2-ITD peptide (H, I) or with B-LCL cells transfected with wild-type or mutant Her-2 sequences (J), and IFN-γ production was measured in the supernatant. (K) The expression of the transferred TCR, measured by staining with a Vβ2-specific antibody, was improved by CRISPR-mediated deletion of the endogenous TCRα constant region gene (TRAC). (L) Tumor and non-adjacent lung were subjected to deep TCR Vβ sequencing and the Her2-ITD specific Vβ was quantitated as a percentage of TCR Vβ templates p=0.004 for enrichment in the tumor relative to lung by Fisher's exact test.

FIGS. 6A and 6B show that multiple exemplary Her2-ITD reactive T cell lines share a common TCR Vβ clonotype. (A) Schematic illustration of Her2 exon 20 insertion (internal tandem duplication (ITD)) adapted from PloS One 12.2 (2017): e0171225. (B) Ten different Her2-ITD-reactive T cell lines derived from patient 1238 were analyzed by TCR Vβ deep sequencing and percentages of TCR Vβ templates (y-axis) are shown for each T cell line.

FIGS. 7A and 7B show that variant allele frequency and mRNA expression did not correlate with immunogenicity of expressed mutations. (A) Mutations identified to be immunogenic and non-immunogenic from the five patients in this series were compared for mRNA expression in TPM defined by the mean expression in the cancer genome atlas database for lung adenocarcinoma with the top and bottom 20% of the distributions removed for patients 1139, 1238, 1490, and 511, and by measured mRNA expression in a patient derived xenograft from patient 1347 (p=0.5 by Mann-Whitney test). (B) A fraction of variant allele sequencing reads for immunogenic and non-immunogenic screened mutations from the five patients, p=0.78 by Mann-Whitney test.

FIG. 8 shows KRAS G12V-specific CD4+ T cell clonotypes derived from the blood of a healthy HLA-DRB1-1104 donor.

### DETAILED DESCRIPTION

In some aspects, the present disclosure provides binding proteins and/or high affinity recombinant TCRs directed to KRAS G12V or Her2-ITD neoantigens. Compositions and recombinant host cells including (i.e., encoding and/or expressing) the binding proteins and/or high affinity recombinant TCRs are also provided. Compositions and recombinant host cells according to the present disclosure are useful to treat a subject having non-small cell lung cancer (NSCLC), colorectal cancer, pancreas cancer, other indications (also referred to herein as a disease or disorder) wherein a KRAS G12V neoantigen is a therapeutic target, and indications wherein a Her2-ITD neoantigen is a therapeutic target. In some embodiments, compositions and recombinant host cells (e.g., immune cells, such as T cells, that are modified to encode and/or express a KRAS G12V-specific binding protein or high affinity recombinant TCR as disclosed herein) with specificity for a KRAS G12V neoantigen are useful to treat a subject having biliary tract cancer. In certain embodiments, compositions and recombinant host cells with specificity for a Her2-ITD neoantigen (e.g., immune cells, such as T cells, that are modified to encode and/or express a Her2-ITD-specific binding protein or high affinity recombinant TCR as disclosed herein) may be used to treat a subject having a disease or disorder associated with the Her2-ITD neoantigen, such as, for example, ovarian cancer or breast cancer. Immunogenic compositions such as, for example, vaccines, as well as related uses are also provided.

It will be readily understood that the embodiments, as generally described herein, are exemplary. The following description of various embodiments is not intended to limit the scope of the present disclosure, but is merely representative of various embodiments. Moreover, the order of steps or actions of certain methods disclosed herein may be changed by those skilled in the art without departing from the scope of the present disclosure. In other words, unless a specific order of steps or actions is required for proper operation of the embodiment, the order or use of specific steps or actions may be modified.

Prior to setting forth this disclosure in more detail, it may be helpful to an understanding thereof to provide definitions of certain terms to be used herein. Additional definitions are set forth throughout this disclosure.

Unless specifically defined otherwise, the technical terms, as used herein, have their normal meaning as understood in the art.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated.

“About,” as used herein, when referring to a measurable value is meant to encompass variations of ±20%, ±10%, ±5%, ±1%, or ±0.1% from the specified or indicated value, range, or structure, unless otherwise indicated.

It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., an a-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” refers to a protein that, in a single chain, has at least two distinct domains, wherein the domains are not naturally found together in a protein. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a protein expressed or produced by a host cell (e.g., a T cell) locates to the cell surface, where the fusion protein is anchored to the cell membrane (e.g., via a transmembrane domain) and comprises an extracellular portion (e.g., containing a binding domain) and an intracellular portion (e.g., containing a signaling domain, effector domain, co-stimulatory domain or combinations thereof).

“Junction amino acids” or “junction amino acid residues” refer to one or more (e.g., about 2-10) amino acid residues between two adjacent motifs, regions, or domains of a polypeptide, such as between a binding domain and an adjacent constant domain or between a TCR chain and an adjacent self-cleaving peptide. Junction amino acids may result from the construct design of a fusion protein (e.g., amino acid residues resulting from the use of a restriction enzyme site during the construction of a nucleic acid molecule encoding a fusion protein).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. If single-stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W. H. Freeman and Company. In certain embodiments, proline shares certain properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. Variant proteins, peptides, polypeptides, and amino acid sequences of the present disclosure can, in certain embodiments, comprise one or more conservative substitutions relative to a reference amino acid sequence.

As understood in the art, “similarity” between two polypeptides is determined by comparing the amino acid sequence and conserved amino acid substitutes thereto of the polypeptide to the sequence of a second polypeptide (e.g., using GENEWORKS™, Align, Clustal™, the BLAST algorithm, or the like).

Variants of polynucleotides and polypeptides of this disclosure are also contemplated. Variant nucleic acid molecules or polynucleotide are at least 70%, 75%, 80%, 85%, 90%, and are preferably at least 90%, 91%, 92%, 93%, 94%, 95%, 96%, 97%, 98%, 99%, or 99.9% A identical to a defined or reference polynucleotide or polypeptide (respectively) as described herein, or that, for a polynucleotide, hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule. For additional details and explanation of stringency of hybridization reactions, see Ausubel, F. M. (1995), Current Protocols in Molecular Biology. John Wiley & Sons, Inc. Moreover, the person skilled in the art may follow the instructions given in the manual Boehringer Mannheim GmbH (1993) The DIG System Users Guide for Filter Hybridization, Boehringer Mannheim GmbH, Mannheim, Germany and in Liebl, W., Ehrmann, M., Ludwig, W., and Schleifer, K. H. (1991) International Journal of Systematic Bacteriology 41: 255-260 on how to identify DNA sequences by means of hybridization.

Variants can also refer to fragments (e.g., a portion resulting from truncation, cleavage, or the like) of a defined or reference sequence, and fragments can be of any length shorter than the length of the defined or reference sequence.

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “active portion” or “functional fragment” or “active fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release). In certain embodiments, a functional portion refers to a “signaling portion” of an effector molecule, effector domain, costimulatory molecule, or costimulatory domain.

In certain embodiments, a variant binding protein or a portion or fragment thereof (e.g., binding domain) can comprise one or more amino acid substitutions relative to a parent or reference binding protein or domain, wherein the one or more amino acid substitutions remove, change, or attenuate a potential undesired feature or characteristic, if present, from the parent or reference binding domain or protein; e.g., an amino acid sequence that is potentially immunogenic, or an amino acid sequence that may provide an undesired glycosylation site, an undesired deamidation site, an undesired oxidation site, an undesired isomerization site, or a reduction in thermodynamic stability, or that may result in mis-pairing or mis-folding in a binding protein (e.g., unpaired cysteine residues in close proximity). Amino acid sequences, patterns, and motifs that may provide for an undesired feature or characteristic are known (see, e.g., Seeliger et al., mAbs 7(3): 505-515 (2015)).

In certain embodiments, an amino acid substitution comprises a substitution to remove a somatic mutation, such as, for example, a reversion to a germ line-encoded amino acid. For example, in certain embodiments, a variant of a reference CDR amino acid sequence, or of a TCR variable domain sequence or TCR constant region sequence, comprises a substitution to remove or attenuate a potential undesired feature or characteristic. It will be understood that such variants are selected so as not to compromise, or substantially compromise, a desired function (e.g., binding specificity and/or affinity for a peptide antigen:HLA complex).

“Sequence identity,” or “percent sequence identity” as used herein, refers to the percentage of amino acid residues in one sequence that are identical with the amino acid residues in another reference polypeptide sequence after aligning the sequences and introducing gaps (e.g., gaps can be introduced in one or both of a first and a second amino acid or nucleic acid sequence for optimal alignment), if necessary, to achieve, in preferred methods, the maximum percent sequence identity, and not considering any conservative substitutions as part of the sequence identity. Further, non-homologous sequences may be disregarded for comparison purposes. The percent sequence identity referenced herein is calculated over the length of the reference sequence, unless indicated otherwise. Within the context of this disclosure, it will be understood that where sequence analysis software is used for analysis, the results of the analysis are based on the “default values” of the program referenced. “Default values” mean any set of values or parameters which originally load with the software when first initialized. For example, percent sequence identity values can be generated using the NCBI BLAST 2.0 software as defined by Altschul, et al. (1997) “Gapped BLAST and PSI-BLAST: a new generation of protein database search programs,” Nucleic Acids Res. 25:3389-3402, with the parameters set to default values. Other programs for determining or calculating sequence alignments and percent identity include, for example, BLASTP, BLASTN, and BLASTX.

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant) or avidity; or an assay measuring phosphorylation or activation of, or by, an immune cell protein such as, for example, Lck, ZAP70, Fyn, or the like, including the assays described herein. The ability of a polypeptide or encoded polypeptide of this disclosure (or a functional variant of the same) to initiate, continue, participate in, propagate, or amplify a cell signaling event or events (e.g., T cell signaling in response to antigen-binding) may be determined by examining the activity, structure, chemical state (e.g., phosphorylation), or interactions of or between the variant polypeptide and an immune cell protein that directly acts (e.g., binds to) therewith, or by examining the activity, localization, structure, expression, secretion, chemical state (e.g., phosphorylation), or interactions of or between other biomolecules known or thought to participate in or be affected by the cell signaling event or events. The ability of a polypeptide or encoded polypeptide of this disclosure (or a functional variant of the same) to initiate, continue, participate in, propagate, or amplify a cell signaling event or events may also be determined by using functional assays of host cell activity, including those described herein for measuring the ability of a host cell to release cytokines, proliferate, selectively kill target cells, or treat a subject having a disease or condition expressing or otherwise associated with an antigen bound by a binding protein of this disclosure.

Variant polypeptides of the present disclosure can, in certain embodiments, include chemical modifications, for example, isotopic labels or covalent modifications such as glycosylation, phosphorylation, acetylation, decarboxylation, citrullination, hydroxylation and the like. Methods to modify polypeptides are known in the art. Modifications are designed so as not to abolish or substantially impair a desired biological activity of the variant.

An “altered domain” or “altered protein” refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, or TCRβ constant domain) of at least 85% (e.g., 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%).

As used herein, the terms “endogenous” or “native” refer to a gene, protein, or activity that is normally present in a host cell.

As used herein, “heterologous,” “non-endogenous,” and “exogenous” refer to any gene, protein, compound, molecule, or activity that is introduced through manipulation (e.g., genetic manipulation). In certain embodiments, heterologous, non-endogenous, or exogenous molecules (e.g., receptors, ligands, etc.) may not be endogenous to a host cell or subject, but instead nucleic acids encoding such molecules may have been added to a host cell by conjugation, transformation, transfection, transduction, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a molecule or activity found in or derived from a host cell, species, or strain. For example, a heterologous, non-endogenous, or exogenous molecule or gene encoding the molecule may be homologous to a native host or host cell molecule or gene that encodes the molecule, respectively, but may have an altered structure, sequence, expression level, or combinations thereof. A non-endogenous molecule may be from the same species, a different species, or a combination thereof.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably-linked” refers to the association of two or more nucleic acid molecules on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” refers to genetic elements that are not closely associated with one another and the function of one does not affect the other.

The term “construct” refers to any polynucleotide that contains a recombinant nucleic acid molecule. A construct may be present in a vector (e.g., a bacterial vector or a viral vector) or may be integrated into a genome. A “vector” is a nucleic acid molecule that is capable of transporting another nucleic acid molecule. Vectors may be, for example, plasm ids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector) or expression of nucleic acid molecules to which they are linked (expression vectors).

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus,” and “vector” are often used interchangeably.

The term “introduced” in the context of inserting a nucleic acid molecule into a cell, means “transfection,” “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous, non-endogenous, or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, a host cell can be modified to express two or more heterologous, non-endogenous, or exogenous nucleic acid molecules encoding desired TCR specific for a KRAS G12V or Her2-ITD neoantigen peptide (e.g., TCRα and TCRβ). When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the terms “host” or “host cell” refer to a cell (e.g., an immune system cell such as, for example, a T cell) or microorganism targeted for genetic modification with a heterologous or exogenous nucleic acid molecule to produce a polypeptide of interest (e.g., KRAS G12V or Her2-ITD-specific binding protein). In certain embodiments, a host cell may optionally already possess or be modified to include other genetic modifications that confer desired properties related or unrelated to biosynthesis of the heterologous or exogenous protein (e.g., inclusion of a detectable marker; deleted, altered or truncated endogenous TCR; increased co-stimulatory factor expression; etc.). Exemplary host cells and types of cells suitable for use as host cells are described further herein.

“T cell receptor” (TCR) refers to an immunoglobulin superfamily member (having a variable binding domain, a constant domain, a transmembrane region, and a short cytoplasmic tail; see, e.g., Janeway, et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like other immunoglobulins, the extracellular portion of TCR chains (e.g., α-chain and β-chain) contain two immunoglobulin domains, a variable domain (e.g., α-chain variable domain or Vα, β-chain variable domain or Vβ; typically amino acids 1 to 116 based on Kabat numbering (Kabat, et al., “Sequences of Proteins of Immunological Interest,” US Dept. Health and Human Services, Public Health Service National Institutes of Health, 1991, 5th ed.)) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also like other immunoglobulins, the variable domains contain complementary determining regions (CDRs) separated by framework regions (FRs) (see, e.g., Jores, et al., Proc. Nat'l Acad. Sci. U.S.A. 57:9138, 1990; Chothia, et al., EMBO J. 7:3745, 1988; see also Lefranc, et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, cat, dog, goat, horse, or other mammal. In certain embodiments, a TCR complex comprises a TCR or a functional portion thereof; a dimer comprising two CD3ζ chains, or functional portions or variants thereof; a dimer comprising a CD3δ chain and a CDε chain, or functional portions or variants thereof; and a dimer comprising a CD3γ chain and a CDε chain, or functional portions or variants thereof, any one or more of which may be endogenous or heterologous to the T cell.

“CD3” is a multi-protein complex of six chains (see, Borst J, et al., J Biol Chem, 258(8):5135-41, 1983 and Janeway, et al., p. 172 and 178, 1999 supra). In mammals, the complex includes a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is thought to allow these chains to associate with positively charged regions of TCR chains. The intracellular tails of the CD3γ, CD3δ, and CD3ε chains each contain a single conserved motif known as an immunoreceptor tyrosine-based activation motif or ITAM, whereas each CD3ζ chain has three. Without being bound by theory, it is believed the ITAMs are important for the signaling capacity of a TCR complex. CD3 as used in the present disclosure may be from various animal species, including human, mouse, rat, or other mammals.

As used herein, “TCR complex” refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain. A “component of a TCR complex,” as used herein, refers to a TCR chain (i.e., TCRα, TCRβ, TCRγ, or TCRδ), a CD3ζ chain (i.e., CD3γ, CD3δ, CD3ε, or CD3ζ) or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Major histocompatibility complex” (MHC) refers to glycoproteins that deliver peptide antigens to a cell surface. MHC class I molecules are heterodimers having a membrane spanning a chain (with three α domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

“CD4” refers to an immunoglobulin co-receptor glycoprotein that assists the TCR in communicating with antigen-presenting cells (see, Campbell & Reece, Biology 909 (Benjamin Cummings, Sixth Ed., 2002); UniProtKB P01730). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHCII α1/β1).

As used herein, the term “CD8 co-receptor” or “CD8” means the cell surface glycoprotein CD8, either as an alpha-alpha homodimer or an alpha-beta heterodimer. The CD8 co-receptor assists in the function of cytotoxic T cells (CD8+) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In humans, there are five (5) different CD8 beta chains (see UniProtKB identifier P10966) and a single CD8 alpha chain (see UniProtKB identifier P01732).

“Chimeric antigen receptor” (CAR) refers to a fusion protein engineered to contain two or more naturally occurring amino acid sequences linked together in a way that does not occur naturally or does not occur naturally in a host cell, which fusion protein can function as a receptor when present on a surface of a cell. CARs of the present disclosure include an extracellular portion comprising an antigen binding domain (i.e., obtained or derived from an immunoglobulin or immunoglobulin-like molecule, such as a scFv or scTCR derived from an antibody or TCR specific for a cancer antigen, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014)). In certain embodiments, a binding protein comprises a CAR comprising an antigen-specific TCR binding domain (see, e.g., Walseng et al., Scientific Reports 7:10713, 2017; the TCR CAR constructs and methods of which are hereby incorporated by reference in their entirety).

The term “variable region” or “variable domain” refers to the domain of a TCR α-chain or β-chain (or γ-chain and δ-chain for γδ TCRs), or of an antibody heavy or light chain, that is involved in binding to antigen. The variable domains of the α-chain and β-chain (Vα and Vβ, respectively) of a native TCR generally have similar structures, with each domain comprising four generally conserved framework regions (FRs) and three CDRs. Variable domains of antibody heavy (VH) and light (VL) chains each also generally comprise four generally conserved framework regions (FRs) and three CDRs. In some instances, variable domains of both of a TCR α-chain or β-chain (or γ-chain and δ-chain for γδ TCRs), or of an antibody heavy or light chain, are involved in binding. In some instances, a variable domain of one of a TCR α-chain or β-chain (or γ-chain and δ-chain for γδ TCRs), or of an antibody heavy or light chain, is involved in binding.

The terms “complementarity determining region,” and “CDR,” are synonymous with “hypervariable region” or “HVR,” and are known in the art to refer to sequences of amino acids within TCR or antibody variable regions, which confer antigen specificity and/or binding affinity and are separated in primary sequence from one another by framework amino acids. In general, there are three CDRs in each variable region (i.e., three CDRs in each of the TCRα-chain and β-chain variable regions; 3 CDRs in each of the antibody heavy chain and light chain variable regions). In the case of TCRs, CDR3 is thought to be the main CDR responsible for recognizing processed antigen. In general, CDR1 and CDR2 mainly, or in some cases, exclusively, interact with the MHC. Variable domain sequences can be aligned to a numbering scheme (e.g., Kabat, EU, International Immunogenetics Information System (IMGT), Contact, and Aho), which can allow equivalent residue positions to be annotated and for different molecules to be compared using Antigen receptor Numbering And Receptor Classification (ANARCI) software tool (2016, Bioinformatics 15:298-300). In certain embodiments of the present disclosure, CDRs are determined using IMGT numbering. IMGT determination of CDRs from a TCR sequence can be achieved using, for example, IMGT V-Quest (imgt.org/IMGTindex/V-QUEST.php). It will be understood that a CDR from a, for example, TCR Vα or Vβ region or domain may have a particular sequence according to a particular numbering scheme, and may have a shorter, longer, or shifted (e.g., partially overlapping) sequence by a different numbering scheme.

“Antigen” or “Ag” as used herein refers to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

A “neoantigen,” as used herein, refers to a host cellular product containing a structural change, alteration, or mutation that creates a new antigen or antigenic epitope that has not previously been observed in the subject's genome (i.e., in a sample of healthy tissue from the subject) or been “seen” or recognized by the host's immune system, which: (a) can be processed by the cell's antigen-processing and transport mechanisms and presented on the cell surface in association with an MHC (e.g., HLA) molecule; and (b) can elicit an immune response (e.g., a cellular (T cell) response). Neoantigens may originate, for example, from coding polynucleotides having alterations (substitution, addition, deletion) that result in an altered or mutated product, or from the insertion of an exogenous nucleic acid molecule or protein into a cell, or from exposure to environmental factors (e.g., chemical, radiological) resulting in a genetic change. Neoantigens may arise separately from a tumor antigen, or may arise from or be associated with a tumor antigen. “Tumor neoantigen” (or “tumor specific neoantigen”) refers to a protein comprising a neoantigenic determinant associated with, arising from, or arising within a tumor cell or plurality of cells within a tumor. Tumor neoantigenic determinants are found on, for example, antigenic tumor proteins or peptides that contain one or more somatic mutations or chromosomal rearrangements encoded by the DNA of tumor cells, as well as proteins or peptides from viral open reading frames associated with virus-associated tumors (e.g., cervical cancers, some head and neck cancers). The terms “antigen” and “neoantigen” are used interchangeably herein when referring to a KRAS antigen comprising a mutation (e.g., G12V) or a HER2-ITD antigen as disclosed herein.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics. Epitopes can be comprised of consecutive amino acids (e.g., a linear epitope), or amino acids from different parts of a protein that are brought into proximity by protein folding (e.g., a discontinuous or conformational epitope), or non-contiguous amino acids that are in close proximity irrespective of protein folding and/or processing by the cellular immune system.

A “binding domain” (also referred to as a “binding region” or “binding moiety”), as used herein, refers to a molecule, such as a peptide, oligopeptide, polypeptide, or protein that possesses the ability to specifically and non-covalently associate, unite, or combine with a target molecule (e.g., KRAS G12V peptide (SEQ ID NO:1, or an immunogenic fragment thereof comprising or consisting of about 7, 8, 9, 10, 11, 12, 13, 14, 15, 16, 27, 28, 19, 20, 21, 22, 23, or 24 contiguous amino acids of SEQ ID NO:1); KRAS G12V peptide:MHC complex, wherein the MHC allele can be DRB1-1101 or DRB1-1104, Her2-ITD (SEQ ID NO:22; or an immunogenic fragment thereof comprising or consisting of at 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO:22), or Her2-ITD peptide:MHC complex, wherein the MHC allele can be DQB1-05:01 or DQB1-05:02). A binding domain includes any naturally occurring, synthetic, semisynthetic, or recombinantly produced binding partner for a biological molecule or other target of interest. In some embodiments, the binding domain is an antigen-binding domain, such as an antibody or TCR or functional binding domain or antigen-binding fragment thereof. Exemplary binding domains include single chain antibody variable regions (e.g., single domain antibodies, sFv, scFv, and Fab), receptor ectodomains (e.g., TNF-α), ligands (e.g., cytokines and chemokines), antigen-binding regions of TCRs, such as single chain TCRs (scTCRs), synthetic polypeptides selected for the specific ability to bind to a biological molecule, aptamers, or single domain antibodies (e.g., camelid or fish-derived single domain antibodies; see, e.g., Arbabi-Ghahroudi M (2017) Front. Immunol. 8:1589).

A “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids, about four to about 20 amino acids, about eight to about 15 amino acids, about 15 to about 25 amino acids, or another suitable number of amino acids. Exemplary linkers include glycine-serine linkers, wherein one or more consecutive glycines are followed by a serine, which sequence may be repeated two, three, four, or more times.

Any binding domain of the present disclosure may be engineered in a single chain format so that the C-terminal end of a first domain is linked by a short peptide sequence to the N-terminal end of a second domain, or vice versa (e.g., in the case of a scTCR, (N)Vβ(C)-linker-(N)Vα(C) or (N)Vα(C)-linker-(N)Vβ(C). In certain embodiments, the binding domain is chimeric, human, or humanized.

As used herein, the term “KRAS G12V-specific binding protein” refers to a protein or polypeptide that specifically binds to and/or that is specific for a KRAS G12V neoantigen. By way of background, KRAS (also called C-K-RAS, CFC2, K-RAS2A, K-RAS2B, K-RAS4A, K-RAS4B, KI-RAS, KRAS1, KRAS2, NS, NS3, RALD, RASK2, K-ras, KRAS proto-oncogene, GTPase, and c-Ki-ras2) is a p21 GTPase that is involved in signal transduction of cell proliferation. Mutations in KRAS that disrupt negative growth signaling can lead to continuous proliferation of the cell. It has been reported that a KRAS G12V mutation is found in 4% of NSCLCs, 10% of colorectal cancers, 30% of pancreas cancers and 8% of ovarian cancers (see Forbes S, et al. Current protocols in human genetics. 2016:10.1.1-.1.37).

In some embodiments, a binding protein or polypeptide binds to KRAS G12V, such as a KRAS G12V peptide complexed with an MHC or HLA molecule, e.g., on a cell surface, with a, or at least about a, particular affinity. A KRAS G12V-specific binding protein may bind to a KRAS G12V neoantigen, a variant thereof, or a fragment thereof. For example, the KRAS G12V-specific binding protein may bind to an amino acid sequence according to SEQ ID NO:1, or to an amino acid sequence having at least 90%, 91%, 92%, 93%, 94%, 95%, 96%, 97%, 98%, 99%, or more sequence identity to SEQ ID NO:1, wherein the residue corresponding to residue 12 of SEQ ID NO:1 is valine (V). In certain embodiments, a KRAS G12V-specific binding protein binds a KRAS G12V-derived peptide:HLA complex (or KRAS G12V-derived peptide:MHC complex) with an affinity that is about the same as, at least about the same as, or is greater than at or about the affinity exhibited by an exemplary KRAS G12V-specific binding protein provided herein, such as any of the KRAS G12V-specific TCRs provided herein, for example, as measured by the same assay. K_{d} can be measured to assess the affinity of a KRAS G12V-specific binding protein.

The term “Her2-ITD-specific binding protein” refers to a protein or polypeptide that specifically binds to and/or that is specific for the Her2-ITD neoantigen. In some embodiments, a protein or polypeptide binds to a Her2-ITD antigen, such as a Her2-ITD neoantigen peptide, when complexed with an MHC or HLA molecule, e.g., on a cell surface, with a, or at least about a, particular affinity. A Her2-ITD-specific binding protein may bind to a Her2-ITD neoantigen, a variant thereof, or a fragment thereof. For example, the Her2-ITD-specific binding protein may bind to an amino acid sequence of SEQ ID NO:22, or an amino acid sequence having at least 90%, 91%, 92%, 93%, 94%, 95%, 96%, 97%, 98%, 99%, or more sequence identity to SEQ ID NO:22. In certain embodiments, a Her2-ITD-specific binding protein binds a Her2-ITD-derived peptide:HLA complex (or Her2-ITD-derived peptide:MHC complex) with an affinity that is about the same as, at least about the same as, or is greater than at or about the affinity exhibited by an exemplary Her2-ITD specific binding protein provided herein, such as any of the Her2-ITD-specific TCRs provided herein, for example, as measured by the same assay. K_{d} can be measured to assess the affinity of a Her2-ITD-specific binding protein.

Assays for assessing affinity or apparent affinity or relative affinity are known. For example, apparent affinity of a TCR for antigen:HLA can be measured by assessing binding to various concentrations of tetramers, for example, by flow cytometry using labeled tetramers. In some examples, apparent K_{d} of a TCR is measured using 2-fold dilutions of labeled tetramers at a range of concentrations, followed by determination of binding curves by non-linear regression, apparent K_{d} being determined as the concentration of ligand that yielded half-maximal binding. In certain embodiments, a KRAS G12V- or Her2-ITD-specific binding protein includes a KRAS G12V- or Her2-ITD-specific immunoglobulin superfamily binding protein or binding portion thereof, respectively.

As used herein, “specifically binds” refers to an association or union of a binding protein (e.g., a T cell receptor or a chimeric antigen receptor) or a binding domain (or a fusion protein thereof), to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample. Binding domains (or fusion proteins thereof) may be classified as “high affinity” binding domains (or fusion proteins thereof) or “low affinity” binding domains (or fusion proteins thereof). “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ at least 10¹² M⁻¹, or at least 10¹³ M⁻¹. “Low affinity” binding domains refer to those binding domains with a Kₐ of up to 10⁷ M⁻¹, up to 10⁶ M⁻¹, or up to 10⁵ M. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domain with stronger binding to a target antigen than a wild type (or parent) binding domain. For example, enhanced affinity may be due to a Kₐ (equilibrium association constant) for the target antigen that is higher than the wild type binding domain, or due to a K_{d} for the target antigen that is less than that of the wild type binding domain, or due to an off-rate (K_{off}) for the target antigen that is less than that of the wild type binding domain. A variety of assays are known for identifying binding domains of the present disclosure that specifically bind a particular target, as well as determining binding domain or fusion protein affinities, such as western blot, ELISA, and BIACORE® analysis (see also, e.g., Scatchard, et al., Ann. N. Y. Acad. Sci. 57:660, 1949; and U.S. Pat. Nos. 5,283,173, 5,468,614, or the equivalent).

The KRAS G12V neoantigen- or Her2-ITD neoantigen-specific binding proteins, TCRs, or domains as described herein, and variants thereof, may be functionally characterized according to any of a large number of art accepted methodologies for assaying host cell activity, including determination of host cell binding, activation or induction and also including determination of host cell responses that are antigen-specific. Examples include determination of host cell proliferation, host cell cytokine release, antigen-specific host cell stimulation, MHC restricted host cell stimulation, cytotoxic T lymphocyte (CTL) activity (e.g., by detecting ⁵¹Cr release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998; see also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); and Green and Reed, Science 281:1309 (1998) and references cited therein).

By way of further illustration, in the case of a host cell that expresses a binding protein of the present disclosure, avidity of the host cell for antigen can be determined by, for example, exposing the host cell to the peptide, or to a peptide:HLA complex (e.g., organized as a tetramer or other multimer), or to an antigen-presenting cell (APC) that presents the peptide to the host cell, optionally in a peptide:HLA complex, and then measuring an activity of the host cell, such as, for example, production or secretion of cytokines (e.g., IFN-γ; TNFα); increased expression of host cell signaling or activation components (e.g., CD137 (4-1BB)); proliferation of the host cell; or killing of the APC (e.g., using a labeled-chromium release assay).

“MHC-peptide tetramer staining” refers to an assay used to detect antigen-specific cells expressing a binding protein comprising a TCR variable domain or binding domain, which assay comprises a tetramer of MHC molecules, each comprising (presenting) an identical peptide having an amino acid sequence that is cognate (e.g., identical or related to) at least one neoantigen (e.g., KRAS G12V or Her2-ITD), wherein the complex is capable of associating with TCRs specific for the cognate neoantigen. Each of the MHC molecules may be tagged with a biotin molecule. Biotinylated MHC/peptide complexes can be multimerized (e.g., tetramerized) by the addition of streptavidin, which can, in some embodiments, be fluorescently labeled. The tetramer may be detected by flow cytometry via the fluorescent label. In certain embodiments, an MHC-peptide tetramer assay is used to detect or select a binding protein or TCR of the instant disclosure. Levels of cytokines may be determined according to methods described herein and practiced in the art, including for example, ELISA, ELISpot, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation, and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

A target molecule, which is specifically bound by a binding domain of the present disclosure, may be found on or in association with a cell of interest (“target cell”). Exemplary target cells include any undesired cell in a subject or sample from a subject, or a cell for research purposes, that expresses an antigen (KRAS G12V; HER2 ITD) of the present disclosure, such as, for example, a cancer cell, a cell associated with an autoimmune disease or disorder or with an inflammatory disease or disorder, and an infectious organism or cell (e.g., bacteria, virus, or virus-infected cell). A cell of an infectious organism, such as a mammalian parasite, is also contemplated as a target cell.

In certain embodiments, any host cell of the present disclosure (e.g., recombinant host cell expressing and/or encoding a heterologous binding protein as provided herein) can be an immune system cell. As used herein, the terms “immune system cell” and “immune cell” refer to any cell of the immune system that originates from a hematopoietic stem cell in the bone marrow, which gives rise to two major lineages, a myeloid progenitor cell (which gives rise to myeloid cells such as monocytes, macrophages, dendritic cells, megakaryocytes, and granulocytes) and a lymphoid progenitor cell (which gives rise to lymphoid cells such as T cells, B cells, and natural killer (NK) cells). Exemplary immune system cells include a CD4+ T cell, a CD8+ T cell, a CD4− CD8− double negative T cell, a stem cell memory T cell, a γδ T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” is an immune system cell that matures in the thymus and produces TCRs. T cells can be naïve (not exposed to antigen; increased expression of CD62L, CCR7, CD28, CD3, CD127, and CD45RA, and decreased expression of CD45RO as compared to TC_{M}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or TC_{M}). Effector T cells (TE) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4+ CD25+ (Foxp3+) regulatory T cells and Treg17 cells, as well as Tr1, Th3, CD8+CD28−, and Qa-1 restricted T cells.

In certain embodiments, a host cell is a human hematopoietic progenitor cell. A “hematopoietic progenitor cell” is a cell derived from hematopoietic stem cells (HSCs) or fetal tissue that is capable of further differentiation into mature cell types (e.g., cells of the T cell lineage). In certain embodiments, CD24^{lo} Lin⁻ CD117⁺ hematopoietic progenitor cells are useful. As defined herein, hematopoietic progenitor cells may include embryonic stem cells, which are capable of further differentiation to cells of the T cell lineage. Hematopoietic progenitor cells may be from various animal species, including human, mouse, rat, or other mammals. A “thymocyte progenitor cell” or “thymocyte” is a hematopoietic progenitor cell present in the thymus.

“Hematopoietic stem cells” or “HSCs” refer to undifferentiated hematopoietic cells that are capable of self-renewal either in vivo, essentially unlimited propagation in vitro, and capable of differentiation to other cell types including cells of the T cell lineage. HSCs may be isolated, for example, but not limited to, from fetal liver, bone marrow, and cord blood.

“Embryonic stem cells,” “ES cells,” or “ESCs” refer to undifferentiated embryonic stem cells that have the ability to integrate into and become part of the germ line of a developing embryo. Embryonic stem cells are capable of differentiating into hematopoietic progenitor cells and any tissue or organ. Embryonic stem cells that are suitable for use herein include cells from the J1 ES cell line, 129J ES cell line, murine stem cell line D3 (American Type Culture Collection), the R1 or E14K cell lines derived from 129/Sv mice, cell lines derived from Balb/c and C57B1/6 mice, and human embryonic stem cells (e.g., from WICELL® Research Institute, WI; or ES cell International, Melbourne, Australia).

“Cells of T cell lineage” refer to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, and CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCRγδ; or mature and functional or activated T cells.

The term “isolated” refers to material that is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” refers to the segment of DNA involved in producing a polypeptide chain. It includes regions preceding and following the coding region “leader and trailer” as well as intervening sequences (introns) between individual coding segments (exons).

As used herein to describe a cell, microorganism, nucleic acid molecule, or vector, the term “recombinant” or “modified” or “engineered” refers to a cell, microorganism, nucleic acid molecule, or vector that has been modified by introduction of an exogenous nucleic acid molecule (e.g., DNA, RNA) or protein, or refers to a cell or microorganism that has been altered such that expression of an endogenous nucleic acid molecule or gene is controlled, deregulated, or constitutive, where such alterations or modifications may be introduced by genetic engineering. Genetic alterations may include, for example, modifications introducing nucleic acid molecules (which may include an expression control element, such as a promoter) encoding one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

Additional definitions are provided throughout the present disclosure.

### Binding Proteins Specific for KRAS G12V Neoantigens

In one aspect, the present disclosure provides binding proteins (e.g., an immunoglobulin superfamily binding protein or a portion thereof) that include a TCR Vα domain and a Vβ domain, wherein the binding protein is configured to bind to, is capable of binding to, and/or is specific for a KRAS G12V neoantigen.

In certain embodiments, a KRAS G12V-specific binding protein is configured to bind to, capable of binding to, or is specific for an MTEYKLVVVVGAVGVGKSALTIQLIQ (SEQ ID NO:1):HLA complex, or a peptide:HLA complex wherein the peptide comprises or consists of about 7, 8, 9, 10, 11, 12, 13, 14, 15, 16, 27, 28, 19, 20, 21, 22, 23, or 24 contiguous amino acids of SEQ ID NO:1). In some embodiments, the HLA comprises DRB1-1101 or DRB1-1104.

In some embodiments, the TCR Vα domain comprises a CDR3 amino acid sequence that is at least about 85% (i.e., at least about 85%, 86%, 87%, 88%, 89%, 90%, 91%, 92%, 93%, 94%, 95%, 96%, 97%, 98%, 99%, 99.5%, or 100%) identical to the amino acid sequence set forth in SEQ ID NO:2 or SEQ ID NO:12. In certain embodiments, the TCR Vα domain CDR3 amino acid sequence comprises or consists of the amino acid sequence set forth in SEQ ID NO:2 or SEQ ID NO:12. In certain embodiments, the TCR Vβ domain comprises a CDR3 amino acid sequence that is at least about 85%, 86%, 87%, 88%, 89%, 90%, 91%, 92%, 93%, 94%, 95%, 96%, 97%, 98%, 99%, 99.5%, or 100% identical to the amino acid sequence set forth in SEQ ID NO:3 or SEQ ID NO:13. In certain embodiments, the TCR Vα domain CDR3 amino acid sequence comprises or consists of the amino acid sequence set forth in SEQ ID NO:3 or SEQ ID NO:13.

In any of the presently disclosed embodiments, a KRAS G12V-specific binding protein comprises a CDR1a amino acid sequence that is at least about 85% identical to the amino acid sequence set forth in SEQ ID NO:48 or 54, a CDR2a amino acid sequence that is at least about 85% identical to the amino acid sequence set forth in to SEQ ID NO:49 or 55, a CDR1β amino acid sequence that is at least about 85% identical to the amino acid sequence set forth in to SEQ ID NO:51 or 57, and/or a CDR2β amino acid sequence that is at least about 85% identical to the amino acid sequence set forth in to SEQ ID NO:52 or 58.

In further embodiments, a KRAS G12V-specific binding protein comprises: CDR1α, CDR2α, CDR3α, CDR1β, CDR2β, and CDR3β amino acid sequences as set forth in SEQ ID NOs:48, 49, 2, 51, 52, and 3, respectively; or as set forth in SEQ ID NOs:54, 55, 12, 57, 58, and 13, respectively.

In certain embodiments, a KRAS G12V-specific binding protein comprises a TCR Vα domain that comprises or consists of an amino acid sequence that is at least about 85%, 86%, 87%, 88%, 89%, 90%, 91%, 92%, 93%, 94%, 95%, 96%, 97%, 98%, 99%, 99.5%, or 100% identical to the amino acid sequence set forth in SEQ ID NO:9 or SEQ ID NO:19. In certain embodiments, a KRAS G12V-specific binding protein comprises a TCR Vα domain that comprises or consists of an amino acid sequence that is at least about 85%, 86%, 87%, 88%, 89%, 90%, 91%, 92%, 93%, 94%, 95%, 96%, 97%, 98%, 99%, 99.5%, or 100% identical to the amino acid sequence set forth in SEQ ID NO:6 or SEQ ID NO:16. In further embodiments, any one or more of the β or α CDR amino acid sequences as provided herein can be present in the Vβ domain and/or the Vα domain, respectively.

In certain embodiments, at least three or four of the complementary determining regions (CDRs) may have no change in sequence, and the CDRs that do have sequence changes may have only up to two amino acid substitutions, up to a contiguous five amino acid deletion, or a combination thereof.

In certain embodiments, a KRAS G12V-specific binding protein comprises a TCR Vα domain and a TCR Vβ domain according to SEQ ID NOS:6 and 9, respectively, or according to SEQ ID NOs:16 and 19, respectively.

In any of the embodiments described herein, a binding protein (e.g., KRAS G12V-specific binding protein; HER2-ITD-specific binding protein as discussed herein) can comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In certain embodiments, a binding protein of this disclosure comprises a mature Vβ domain, a mature Vα domain, or both. In some embodiments, a binding protein of this disclosure comprises a mature TCR β-chain, a mature TCR α chain, or a mature TCR β-chain and a mature TCR α chain.

Exemplary binding proteins and fusion proteins of this disclosure expressed by a cell may include a signal peptide (e.g., as binding pre-proteins), and the cell may remove the signal peptide to generate a mature binding protein. In certain embodiments, a binding protein comprises two components, such as an α chain and a β chain, which can associate on the cell surface to form a functional binding protein. The two associated components may comprise mature proteins.

A signal or leader peptide can, in some embodiments, comprise or consist of an amino acid sequence that is at least about 85% (i.e., 85%, 86%, 87%, 88%, 89%, 90%, 91%, 92%, 93%, 94%, 95%, 96%, 97%, 98%, 99%, 99.5%, or 100%) identical to the amino acid sequence set forth in any one of SEQ ID NOs:50, 53, 56, or 59. However, it will be understood that any of the presently disclosed TCRVα and TCRVβ domains, or a binding protein comprising the same, may lack an exemplary signal or leader peptide sequence, or can comprise a different signal or leader peptide sequence.

Accordingly, it will be understood that the present disclosure contemplates KRAS G12V-specific binding proteins that comprise TCRVα and/or TCRVβ domains wherein, for example, the amino acid sequence contained within SEQ ID NO:6, 9, 16, or 19 that corresponds to SEQ ID NO: 50, 53, 56, or 59, respectively, may be absent.

In certain embodiments, a KRAS G12V-specific binding protein comprises a TCR Vα domain having at least about 85% (i.e., 85%, 86%, 87%, 88%, 89%, 90%, 91%, 92%, 93%, 94%, 95%, 96%, 97%, 98%, 99%, 99.5%, or 100%) identity to, comprising, or consisting of the amino acid sequence set forth in SEQ ID NO:68 or 72, and/or comprises a TCR Vβ domain having at least about 85% identity to, comprising, or consisting of the amino acid sequence set forth in SEQ ID NO:66 or 70.

In certain embodiments, a binding protein comprises a TCR variable domain comprising an amino acid sequence encoded by a human TCR V, D, and/or J allele. By way of background, during lymphocyte development, Vα exons are assembled from different variable and joining gene segments (V-J), and Vβ exons are assembled from different variable, diversity, and joining gene segments (V-D-J). The TCRα chromosomal locus has 70-80 variable gene segments and 61 joining gene segments. The TCRβ chromosomal locus has 52 variable gene segments, and two separate clusters of each containing a single diversity gene segment, together with six or seven joining gene segments. Functional Vα and Vβ gene exons are generated by the recombination of a variable gene segment with a joining gene segment for Vα, and a variable gene segment with a diversity gene segment and a joining gene segment for Vβ. Nucleotide and amino acid sequences according to TCR gene segments of various alleles are known in the art and are can be found on the ImMunoGeneTics website; for example, at imgt.org/IMGTrepertoire/LocusGenes/listIG_TR/TR/human/Hu_TRgroup.html.

It will be understood that while a polynucleotide encoding a binding protein can comprise a same nucleotide sequence according to a TCR gene segment as disclosed herein, any nucleotide sequence that encodes the amino acid sequence of the referenced gene segment may be used.

In any of the herein disclosed embodiments, the TCR Vα domain of a KRAS G12V-specific binding protein comprises an amino acid sequence (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 43, 44, 45, 46, 47, 48, 49, 50, 51, 52, 53, 54, 55, 56, 57, 58, 59, or 60 consecutive amino acids, or more) according to TRAV8-3 or TRAV8-1, or an amino acid sequence that is at least 85% identical thereto. In certain embodiments, the TCR V_{β} domain of a KRAS G12V-specific comprises an amino acid sequence of TRBV30 or TRBV12-4. Nucleotide and amino acid sequences of human T cell receptor variable region alleles (e.g., TRAV, TRBV, TRAJ, TRBJ, TRBD), including of the alleles provided herein, are known and are available, for example, through the IMGT (ImMunoGeneTics) Information System®; e.g., at imgt.org/IMGTrepertoire/Proteins/alleles/list_alleles.php?species=Homo %20sa piens&group=TRAV; at imgt.org/IMGTrepertoire/Proteins/alleles/list_alleles.php?species=Homo %20sa piens&group=TRBV; at imgt.org/IMGTrepertoire/Proteins/alleles/list_alleles.php?species=Homo %20sa piens&group=TRAJ; at imgt.org/IMGTrepertoire/Proteins/alleles/list_alleles.php?species=Homo %20sa piens&group=TRBJ; and at imgt.org/IMGTrepertoire/Proteins/alleles/list_alleles.php?species=Homo %20sa piens&group=TRBD.

In some embodiments, a KRAS G12V-specific binding protein comprises an amino acid sequence encoded by a TCR α-chain joining (Jα) domain gene segment and an amino acid sequence encoded by TCR β-chain joining (Jβ) gene segment. A TCR Jα domain can comprise an amino acid sequence according to TRAJ13 or TRAJ38, or an amino acid sequence that is at least 85% identical thereto. A TCR Jβ domain can comprise an amino acid sequence according to TRBJ2-4 or TRBJ2-3, or an amino acid sequence that is at least 85% identical thereto.

These human T cell receptor variable domain allele polynucleotide and amino acid sequences are incorporated by reference herein.

In any of the presently disclosed embodiments (i.e., KRAS G12V-specific binding protein; Her2-ITD-specific binding protein), a binding protein can further comprise a TCR β chain constant domain (Cβ), a TCR α chain constant domain (Cα), or both. Exemplary amino acid sequences of human TCR Cα and Cβ can be found at, for example, UniProtKb P01848 (Cα) and UniProtKb P01850 and A0A5B9 (Cβ). Exemplary amino acid sequences of murine TCR constant regions can be found at UniProtKb A0A0A6YWV4, A0A075B5J4, and A0A075B5J3. These amino acid sequences are incorporated by reference herein.

In any of the presently disclosed embodiments (i.e., KRAS G12V-specific binding protein; Her2-ITD-specific binding protein), the binding protein further comprises a Cβ and a Cα, wherein the Vβ and the Cβ together comprise a TCR β chain, and wherein the Vα and the Cα together comprise a TCR α chain, and wherein the TCR β chain and the TCR α chain are capable of associating to form a dimer.

In further embodiments, a TCR Cβ comprises a cysteine amino acid in place of a native serine at amino acid position 57 (e.g., GV(S→C)TD) and a TCR Cα comprises a cysteine amino acid in place of a native threonine at amino acid position 48 (e.g., DK(T→C)VL; see. e.g., Cohen et al., Cancer Res. 67(8):3898-3903 (2007)).

In certain embodiments, a TCR Cα has at least about 85% (i.e., 85%, 86%, 87%, 88%, 89%, 90%, 91%, 92%, 93%, 94%, 95%, 96%, 97%, 98%, 99%, 99.5%, or 100%) identity to, comprises, or consists of the amino acid sequence set forth in SEQ ID NO:67 or 71. In certain embodiments, a TCR Cβ has at least about 85% identity to, comprises, or consists of the amino acid sequence set forth in SEQ ID NO:69 or 73.

Also contemplated are binding proteins that comprise a TCRα chain and/or a TCRβ chain having at least 85% identity to an amino acid sequence comprised in SEQ ID NO:11 or 21, respectively wherein the amino acid sequence according to SEQ ID NO: 50, 53, 56, or 59, respectively, may be absent. Such binding proteins comprise a “mature” TCRα and/or TCRβ chain.

In certain embodiments, a KRAS G12V-specific binding protein (or a HER2-ITD specific binding protein) may be a TCR, a chimeric antigen receptor, or an antigen-binding fragment of a TCR. In certain embodiments, the TCR, the chimeric antigen receptor, or the antigen-binding fragment of the TCR may be chimeric, humanized, or human. In further embodiments, the antigen-binding fragment of the TCR comprises or consists of a single-chain TCR (scTCR).

Also provided herein are high affinity recombinant TCRs that are configured to bind to, are capable of binding to, and/or are specific for a KRAS G12V neoantigen. A high affinity recombinant TCR can comprise a Vα domain that is at least about 85% (i.e., at least about 85%, 86%, 87%, 88%, 89%, 90%, 91%, 92%, 93%, 94%, 95%, 96%, 97%, 98%, 99%, 99.5%, or 100%) identical to an amino acid sequence of SEQ ID NO:6, 9, 16, or 19. In some embodiments, a high affinity recombinant TCR comprises a TCR Vα domain having at least about 85% identity to, comprising, or consisting of the amino acid sequence set forth in SEQ ID NO:68 or 72, and/or comprises a TCR Vβ domain having at least about 85% identity to, comprising, or consisting of the amino acid sequence set forth in SEQ ID NO:66 or 70.

In some embodiments, the TCR Vα domain comprises a CDR3 amino acid sequence that is at least about 85% (i.e., at least about 85%, 86%, 87%, 88%, 89%, 90%, 91%, 92%, 93%, 94%, 95%, 96%, 97%, 98%, 99%, 99.5%, or 100%) identical to the amino acid sequence set forth in SEQ ID NO:2 or SEQ ID NO:12. In certain embodiments, the TCR Vα domain CDR3 amino acid sequence comprises or consists of the amino acid sequence set forth in SEQ ID NO:2 or SEQ ID NO:12. In certain embodiments, the TCR Vβ domain comprises a CDR3 amino acid sequence that is at least about 85%, 86%, 87%, 88%, 89%, 90%, 91%, 92%, 93%, 94%, 95%, 96%, 97%, 98%, 99%, 99.5%, or 100% identical to the amino acid sequence set forth in SEQ ID NO:3 or SEQ ID NO:13. In certain embodiments, the TCR Vα domain CDR3 amino acid sequence comprises or consists of the amino acid sequence set forth in SEQ ID NO:3 or SEQ ID NO:13.

In certain embodiments, at least three or four of the complementary determining regions (CDRs) may have no change in sequence, and the CDRs that do have sequence changes may have only up to two amino acid substitutions, up to a contiguous five amino acid deletion, or a combination thereof.

In any of the presently disclosed embodiments, a KRAS G12V-specific high affinity recombinant TCR comprises a CDR1α amino acid sequence according to SEQ ID NO:48 or 54, a CDR2α amino acid sequence according to SEQ ID NO:49 or 55, a CDR1β amino acid sequence according to SEQ ID NO:51 or 57, and/or a CDR2β amino acid sequence according to SEQ ID NO:52 or 58.

In further embodiments, a KRAS G12V-specific high affinity recombinant TCR comprises: CDR1α, CDR2α, CDR3α, CDR1β, CDR2β, and CDR3β amino acid sequences as set forth in SEQ ID NOs:48, 49, 2, 51, 52, and 3, respectively; or as set forth in SEQ ID NOs:54, 55, 12, 57, 58, and 13, respectively.

In any of the presently disclosed embodiments, embodiments, a KRAS G12V-specific binding protein or high affinity recombinant TCR is capable of binding to an MTEYKLVVVGAVGVGKSALTIQLIQ (SEQ ID NO:1):DRB1-1101 or (SEQ ID NO:1):DRB1-1104 complex, or a peptide:DRB1-1101 or DRB1-1104 complex wherein the peptide comprises or consists of about 7, 8, 9, 10, 11, 12, 13, 14, 15, 16, 27, 28, 19, 20, 21, 22, 23, or 24 contiguous amino acids of SEQ ID NO:1.

In any of the presently disclosed embodiments, a KRAS G12V-specific binding protein or high affinity recombinant TCR can bind to an MTEYKLVVVGAVGVGKSALTIQLIQ (SEQ ID NO:1):DRB1-1101 or (SEQ ID NO:1):DRB1-1104 complex, or a peptide:DRB1-1101 or DRB1-1104 complex wherein the peptide comprises or consists of about 7, 8, 9, 10, 11, 12, 13, 14, 15, 16, 27, 28, 19, 20, 21, 22, 23, or 24 contiguous amino acids of SEQ ID NO:1, on a cell surface independent or in the absence of CD8 and/or CD4.

### Binding Proteins Specific for Her2-ITD Neoantigens

Another aspect of the present disclosure is directed to a binding protein including a TCR Vα domain and a Vβ domain, wherein the binding protein is configured to bind to, is capable of binding to, and/or is specific for a Her2-ITD neoantigen.

In some embodiments, a TCR Vα domain of a Her2-ITD-specific binding protein comprises a CDR3 amino acid sequence that is at least about 85% (i.e., at least about 85%, 86%, 87%, 88%, 89%, 90%, 91%, 92%, 93%, 94%, 95%, 96%, 97%, 98%, 99%, 99.5%, or 100%) identical to the amino acid sequence of SEQ ID NO:23. In certain embodiments, a TCR Vβ domain comprises a CDR3 amino acid sequence that is at least about 85%, 86%, 87%, 88%, 89%, 90%, 91%, 92%, 93%, 94%, 95%, 96%, 97%, 98%, 99%, 99.5%, or 100% identical to the amino acid sequence of SEQ ID NO:24. In certain embodiments, the TCR Vα domain comprises a CDR3 amino acid sequence as set forth in SEQ ID NO:23 and the TCR Vβ domain comprises a CDR3 amino acid sequence as set forth in SEQ ID NO:24.

In certain embodiments, a Her2-ITD-specific binding protein comprises a TCR Vα CDR1 according to SEQ ID NO:60, a TCR Vα CDR2 according to SEQ ID NO:61, a TCR Vβ CDR1 according to SEQ ID NO:63, and/or a TCR Vβ CDR2 according to SEQ ID NO:64.

In certain embodiments, Her2-ITD-specific binding protein comprises TCR Vα CDRs 1-3 and TCR Vβ CDRs 1-3 according to SEQ ID NOs:60, 61, 23, 63, 64, and 24, respectively.

In any of the presently disclosed embodiments, a Her-ITD-specific binding protein may be configured to bind to, is capable of binding to, and/or is specific for an SPKANKEILDEAYVMAYVMAGVGSPYVSRLLG (SEQ ID NO:22):HLA complex, or a peptide:HLA complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO:22. In certain embodiments, the HLA comprises DQB1-05:01 or DQB1-05:02. In any of the presently disclosed embodiments, a Her-ITD-specific binding protein can bind to an SPKANKEILDEAYVMAYVMAGVGSPYVSRLLG (SEQ ID NO:22):HLA complex, or to a peptide:HLA complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO:22, on a cell surface independent or in the absence of CD8 and/or CD4.

In any of the presently disclosed embodiments, a Her2-ITD-specific binding protein comprises a TCR Vα domain that is at least about 85%, 86%, 87%, 88%, 89%, 90%, 91%, 92%, 93%, 94%, 95%, 96%, 97%, 98%, 99%, 99.5%, or 100% identical to the amino acid sequence of SEQ ID NO:27. In some embodiments, the binding protein comprises a Vβ domain that is at least about 85%, 86%, 87%, 88%, 89%, 90%, 91%, 92%, 93%, 94%, 95%, 96%, 97%, 98%, 99%, 99.5%, or 100% identical to the amino acid sequence of SEQ ID NO:30. In certain embodiments, at least three or four of the CDRs of the binding protein comprise no change in sequence, and the CDRs that do have sequence changes may have only up to two amino acid substitutions, up to a contiguous five amino acid deletion, or a combination thereof.

In certain embodiments, a Her2-ITD-specific binding protein comprises a TCR Vα domain that comprises or consists of an amino acid sequence having at least 85% identity to the amino acid sequence set forth in SEQ ID NO:74, and/or a TCR Vβ domain that comprises or consists of an amino acid sequence having at least 85% identity to the amino acid sequence set forth in SEQ ID NO:76.

In certain embodiments, at least three or four of the CDRs of the binding protein comprise no change in sequence, and the CDRs that do have sequence changes may have only up to two amino acid substitutions, up to a contiguous five amino acid deletion, or a combination thereof.

In certain embodiments, the TCR Vα domain of a Her2-ITD-specific binding protein comprises an amino acid sequence according to TRAV8-6, or an amino acid sequence that is at least 85% identical thereto. In certain embodiments, the TCR Vβ domain of a Her2-ITD-specific binding protein comprises an amino acid sequence according to TRBV20.

In certain embodiments, the binding protein comprises, or further comprises, an amino acid sequence encoded by a TCR Jα domain gene segment, or an amino acid sequence that is at least 85% identical thereto, and an amino acid sequence encoded by a TCR Jβ domain gene segment, or an amino acid sequence that is at least 85% identical thereto. A J_{α} domain can comprise an amino acid sequence according to TRAJ34. A J_{β} domain can comprise an amino acid sequence according to TRBJ2-5, or a sequence that is at least 85% identical thereto.

In certain embodiments, the binding protein further comprises a Cα amino acid sequence having at least 85% identity to the amino acid sequence set forth in SEQ ID NO:75, and/or a Cβ amino acid sequence having at least 85% identity to the amino acid sequence set forth in SEQ ID NO:77.

In certain embodiments, the binding protein comprises a Cβ and a Cα, wherein the Vβ and the Cβ comprise a TCR β chain, and wherein the Vα and the Cα comprise a TCR α chain, and wherein the TCR β chain and the TCR α chain are capable of associating to form a dimer.

In any of the presently disclosed embodiments, a Her2-ITD-specific binding protein may be or comprise a TCR, a chimeric antigen receptor, or an antigen-binding fragment of a TCR. In certain embodiments, the TCR, the chimeric antigen receptor, or the antigen-binding fragment of the TCR is chimeric, humanized, or human. In some embodiments, the antigen-binding fragment of the TCR comprises a scTCR.

Another aspect of the disclosure is directed to a high affinity recombinant TCR that is configured to bind to, capable of binding to, or specific for a Her2-ITD neoantigen. In certain embodiments, the high affinity recombinant TCR comprises an α-chain including a Vα domain having an amino acid sequence that is at least about 85%, 86%, 87%, 88%, 89%, 90%, 91%, 92%, 93%, 94%, 95%, 96%, 97%, 98%, 99%, 99.5%, or 100% identical to the amino acid sequence of SEQ ID NO:74. Furthermore, in any of the presently disclosed embodiments, the TCR is capable of binding to an SPKANKEILDEAYVMAYVMAGVGSPYVSRLLG (SEQ ID NO:22):DQB1-05:01 or (SEQ ID NO:22):DQB1-05:02 complex, or to a peptide:DQB1-05:01 or peptide:DQB1-05:02 complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO:22, on a cell surface independent or in the absence of CD8 and/or CD4.

In certain embodiments, the high affinity recombinant TCR comprises a p-chain including a Vβ domain having an amino acid sequence that is at least about 85%, 86%, 87%, 88%, 89%, 90%, 91%, 92%, 93%, 94%, 95%, 96%, 97%, 98%, 99%, 99.5%, or 100% identical to the amino acid sequence of SEQ ID NO:76. In in any of the presently disclosed embodiments, the TCR is capable of binding to an SPKANKEILDEAYVMAYVMAGVGSPYVSRLLG (SEQ ID NO:22):DQB1-05:01 or DQB1-05:02 complex, or to a peptide:DQB1-05:01 or peptide:DQB1-05:02 complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO:22, on a cell surface independent or in the absence of CD8 and/or CD4.

In certain embodiments, a Her2-ITD-specific high affinity recombinant TCR comprises a CDR1α, CDR2α, CDR3α, CDR1β, CDR2β, and/or CDR3β according to the exemplary Her2-ITD CDR sequences set forth herein, or CDRs having at least 85% identity thereto. In certain embodiments, a Her2-ITD-specific TCR comprises a TCR Vα CDR1 according to SEQ ID NO:60, a TCR Vα CDR2 according to SEQ ID N0:61, a TCR Vβ CDR1 according to SEQ ID NO:63, and/or a TCR Vβ CDR2 according to SEQ ID NO:64.

In certain embodiments, Her2-ITD-specific TCR comprises TCR Vα CDRs 1-3 and TCR Vβ CDRs 1-3 according to SEQ ID NOs:60, 61, 23, 63, 64, and 24, respectively.

In any of the presently disclosed embodiments, a KRAS G12V-specific or Her2-ITD-specific binding protein or high affinity recombinant TCR can be provided in soluble form (see, e.g., Walseng et al., PLoS One doi:10.1371/journal.pone.0119559 (2015)), optionally conjugated to a cytotoxic agent and/or a detectable agent. Methods useful for isolating and purifying recombinantly produced soluble TCR, by way of example, may include obtaining supernatants from suitable host cell/vector systems that secrete the recombinant soluble TCR into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble TCR described herein include batch cell culture, which is monitored and controlled to maintain appropriate culture conditions. Purification of the soluble TCR may be performed according to methods described herein and known in the art and that comport with laws and guidelines of domestic and foreign regulatory agencies.

Another aspect of the disclosure is directed to a composition including a binding protein or high affinity recombinant TCR as described above. The composition may further include a pharmaceutically acceptable carrier, diluent, and/or excipient, as described further herein.

### Immunogenic Compositions

Also provided herein are immunogenic compositions (e.g., for use in a vaccine). In certain embodiments, an immunogenic composition comprises a peptide having an amino acid sequence that is at least about 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% identical to MTEYKLVVV GAVGVGKSALTIQLIQ (SEQ ID NO:1) or SPKANKEILDEAYVMAYVMAGVGS PYVSRLLG (SEQ ID NO:22), or an immunogenic fragment thereof.

In some embodiments, an immunogenic composition comprises an isolated peptide that can, or that is capable of, eliciting an antigen-specific T-cell response to KRAS G12V. The isolated peptide can comprise or be contained in a polypeptide of no more than 25, 24, 23, 22, 21, 20, 19, 18, 17, 16, 15, 14, 13, 12, 11, 10, 9, 8, or 7 amino acids. Furthermore, the polypeptide can include a sequence of at least 7, 8, 9, 10, 11, 12, 13, 14, 15, 16, 17, 18, 19, 20, 21, 22, 23, 24, or 25 contiguous amino acids from the KRAS G12V amino acid sequence set forth in SEQ ID NO:1.

In some embodiments, an immunogenic composition comprises an isolated polypeptide that can, or that is capable of, eliciting an antigen-specific T-cell response to a Her2-ITD antigen. The isolated peptide comprise or be contained a polypeptide of no more than 32, 31, 30, 29, 28, 27, 26, 25, 24, 23, 22, 21, 20, 19, 18, 17, 16, 15, 14, 13, 12, 11, 10, 9, 8, or 7 amino acids. Furthermore, the polypeptide can include a sequence of at least 7, 8, 9, 10, 11, 12, 13, 14, 15, 16, 17, 18, 19, 20, 21, 22, 23, 24, 25, 26, 27, 28, 29, 30, 31, or 32 contiguous amino acids from the Her2-ITD amino acid sequence set forth in SEQ ID NO:22.

In some embodiments, the immunogenic composition further comprises a pharmaceutically acceptable carrier, discussed further herein. The pharmaceutically acceptable carrier may be a non-naturally occurring pharmaceutically acceptable carrier. In certain embodiments, the non-naturally occurring pharmaceutically acceptable carrier may include a cream, emulsion, gel, liposome, nanoparticle, or ointment. In some other embodiments, the vaccine may include an immuno-effective amount of an adjuvant such as poly-ICLC, CpG, GM-CSF, or alum.

### Polynucleotides, Vectors, and Host Cells

Also provided are polynucleotides that encode a binding protein, high-affinity recombinant TCR, immunogenic composition, or a functional fragment or portion thereof, as disclosed herein. It will be appreciated by those of ordinary skill in the art that, due to the degeneracy of the genetic code, there are numerous nucleotide sequences that encode a binding protein, TCR, or immunogenic composition as described herein. Some such polynucleotides can bear limited or minimal sequence identity to the nucleotide sequence of a native, original, or identified polynucleotide sequence. Nonetheless, polynucleotides that vary due to differences in codon usage are expressly contemplated by the present disclosure. In certain embodiments, sequences that have been codon-optimized for expression in a mammalian host cell are specifically contemplated. Codon optimization can be performed using known techniques and tools, e.g., using the GenScript® OptimiumGene™ tool. Codon-optimized sequences include sequences that are partially codon-optimized (i.e., at least one codon is optimized for expression in the host cell) and those that are fully codon-optimized. Codon optimization for expression in certain immune host cells is disclosed in, for example, Scholten et al., Clin. Immunol. 119:135, 2006.

In some embodiments, a single polynucleotide encodes a binding protein as described herein, or, alternatively, the binding protein may be encoded by more than one polynucleotide. In other words, components or portions of a binding protein may be encoded by two or more polynucleotides, which may be contained on a single nucleic acid molecule or may be contained on two or more nucleic acid molecules.

In certain embodiments, a polynucleotide encoding two or more components or portions of a binding protein or TCR of the present disclosure comprises the two or more coding sequences operatively associated in a single open reading frame. Such an arrangement can advantageously allow coordinated expression of desired gene products, such as, for example, contemporaneous expression of alpha and beta chains of a TCR, such that they are produced in about a 1:1 ratio. In certain embodiments, two or more substituent gene products of a binding protein of this disclosure, such as a TCR (e.g., alpha and beta chains), are expressed as separate molecules and associate post-translationally. In further embodiments, two or more substituent gene products of a binding protein of this disclosure are expressed as a single peptide with the parts separated by a cleavable or removable segment. For instance, self-cleaving peptides useful for expression of separable polypeptides encoded by a single polynucleotide or vector are known in the art and include, for example, a Porcine teschovirus-1 2A (P2A) peptide, a Thoseaasigna virus 2A (T2A) peptide, an Equine rhinitis A virus (ERAV) 2A (E2A) peptide, and a Foot-and-Mouth disease virus 2A (F2A) peptide. Exemplary self-cleaving peptides (also referred to as “ribosomal skip elements”) include those comprising or consisting of an amino acid sequence as set forth in any one of SEQ ID NOs:35-38.

Accordingly, in certain embodiments, a heterologous polynucleotide encoding a TCR α-chain and a heterologous polynucleotide encoding a TCR β-chain are contained in a single open reading frame, wherein the single open reading frame further comprises a polynucleotide encoding a self-cleaving peptide disposed between the α-chain-encoding polynucleotide and the β-chain-encoding polynucleotide. It will be understood that either orientation (e.g., β-chain-encoding polynucleotide-self-cleaving peptide-α-chain-encoding polynucleotide; α-chain-encoding polynucleotide-self-cleaving peptide-β-chain-encoding polynucleotide) is contemplated. Exemplary amino acid sequences of such encoded binding proteins are provided in SEQ ID NOs:11, 20, and 32.

In certain embodiments, a polynucleotide of the present disclosure comprises or consists of a polynucleotide having at least about 70%, 75%, 80%, 85%, 90%, 91%, 92%, 93%, 94%, 95%, 96%, 97%, 98%, 99%, 99.9%, or 100% identity to the nucleotide sequence set forth in any one of SEQ ID NOs:4, 5, 7, 8, 10, 14, 15, 17, 18, 20, 25, 26, 28, 29, or 31.

Isolated or recombinant nucleic acid molecules encoding a binding protein or high affinity recombinant TCR specific for KRAS G12V or Her2-ITD as described herein may be produced and prepared according to various methods and techniques of the molecular biology or polypeptide purification arts.

In further embodiments, a binding protein or TCR is expressed as part of a transgene construct that encodes, and/or a host immune cell can further encode: one or more additional accessory protein, such as a safety switch protein; a tag, a selection marker; a CD8 co receptor β chain; a CD8 co-receptor α chain or both; or any combination thereof. Polynucleotides and transgene constructs useful for encoding and expressing binding proteins and accessory components (e.g., one or more of a safety switch protein, a selection marker, CD8 co-receptor β-chain, or a CD8 co-receptor α-chain) are described in PCT application PCT/US2017/053112, the polynucleotides, transgene constructs, and accessory components, including the nucleotide and amino acid sequences, of which are hereby incorporated by reference. It will be understood that any or all of a binding protein of the present disclosure, a safety switch protein, a tag, a selection marker, a CD8 co-receptor β chain, or a CD8 co-receptor α-chain may be encoded by a single nucleic acid molecule or may be encoded by polynucleotide sequences that are, or are present on, separate nucleic acid molecules.

Exemplary safety switch proteins include, for example, a truncated EGF receptor polypeptide (huEGFRt) that is devoid of extracellular N terminal ligand binding domains and intracellular receptor tyrosine kinase activity, but that retains its native amino acid sequence, has type I transmembrane cell surface localization, and has a conformationally intact binding epitope for pharmaceutical-grade anti-EGFR monoclonal antibody, cetuximab (Erbitux) tEGF receptor (tEGFr; Wang et al., Blood 118:1255-1263, 2011); a caspase polypeptide (e.g., iCasp9; Straathof et al., Blood 105:4247-4254, 2005; Di Stasi et al., N. Engl. J. Med. 365:1673-1683, 2011; Zhou and Brenner, Exp. Hematol. pii:S0301-472X(16)30513-6. doi:10.1016/j.exphem.2016.07.011), RQR8 (Philip et al., Blood 124:1277-1287, 2014); a 10-amino-acid tag derived from the human c-myc protein (Myc) (Kieback et al., Proc. Natl. Acad. Sci. USA 105:623-628, 2008); and a marker/safety switch polypeptide, such as RQR (CD20+CD34; Philip et al., 2014).

Other accessory components useful for modified immune cells of the present disclosure comprise a tag or selection marker that allows the cells to be identified, sorted, isolated, enriched, or tracked. For example, marked immune cells having desired characteristics (e.g., an antigen-specific TCR and a safety switch protein) can be sorted away from unmarked cells in a sample and more efficiently activated and expanded for inclusion in a product of desired purity.

As used herein, the term “selection marker” comprises a nucleic acid construct (and the encoded gene product) that confers an identifiable change to a cell permitting detection and positive selection of immune cells transduced with a polynucleotide comprising a selection marker. RQR is a selection marker that comprises a major extracellular loop of CD20 and two minimal CD34 binding sites. In some embodiments, an RQR-encoding polynucleotide comprises a polynucleotide that encodes the 16-amino-acid CD34 minimal epitope. In some embodiments, the CD34 minimal epitope is incorporated at the amino terminal position of a CD8 co-receptor stalk domain (Q8). In further embodiments, the CD34 minimal binding site sequence can be combined with a target epitope for CD20 to form a compact marker/suicide gene for T cells (RQR8) (Philip et al., 2014, incorporated by reference herein). This construct allows for the selection of immune cells expressing the construct, with for example, CD34 specific antibody bound to magnetic beads (Miltenyi) and that utilizes clinically accepted pharmaceutical antibody, rituximab, that allows for the selective deletion of a transgene expressing engineered T cell (Philip et al., 2014).

Further exemplary selection markers also include several truncated type I transmembrane proteins normally not expressed on T cells: the truncated low-affinity nerve growth factor, truncated CD19, and truncated CD34 (see for example, Di Stasi et al., N. Engl. J. Med. 365:1673-1683, 2011; Mavilio et al., Blood 83:1988-1997, 1994; Fehse et al., Mol. Ther. 1:448-456, 2000; each incorporated herein in their entirety). A useful feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non signaling domains or various proteins (e.g., CD19, CD34, LNGFR) also can be employed. Any selection marker may be employed and should be acceptable for Good Manufacturing Practices. In certain embodiments, selection markers are expressed with a polynucleotide that encodes a gene product of interest (e.g., a binding protein of the present disclosure, such as a TCR or CAR). Further examples of selection markers include, for example, reporters such as GFP, EGFP, β-gal or chloramphenicol acetyltransferase (CAT). In certain embodiments, a selection marker, such as, for example, CD34 is expressed by a cell and the CD34 can be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34.

In certain embodiments, a selection marker comprises an RQR polypeptide, a truncated low-affinity nerve growth factor (tNGFR), a truncated CD19 (tCD19), a truncated CD34 (tCD34), or any combination thereof.

Also provided are expression vectors comprising a polynucleotide according to the present disclosure. Any suitable expression vector, including an exemplary expression vector as disclosed herein, may be used. Furthermore, the expression vector may be configured to or capable of delivering the polynucleotide to a host cell.

A typical vector may include a nucleic acid molecule capable of transporting another nucleic acid to which it has been linked, or which is capable of replication in a host organism. As discussed herein, some examples of vectors include plasmids, viral vectors, cosmids, and others. Some vectors may be capable of autonomous replication in a host cell into which they are introduced (e.g., bacterial vectors having a bacterial origin of replication and episomal mammalian vectors), whereas other vectors may be integrated into the genome of a host cell upon introduction into the host cell and thereby replicate along with the host genome. Additionally, some vectors are capable of directing the expression of genes to which they are operatively linked (these vectors may be referred to as “expression vectors”). According to related embodiments, it is further understood that, if one or more agents (e.g., polynucleotides encoding immunoglobulin superfamily binding proteins or high affinity recombinant TCRs specific for KRAS G12V or Her2-ITD, or variants thereof, as described herein) is co-administered to a subject, that each agent may reside in separate or the same vectors, and multiple vectors (each containing a different agent or the same agent) may be introduced to a cell or cell population or administered to a subject.

Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, and cytomegalovirus), and poxvirus (e.g., vaccinia, fowlpox, and canarypox). Other viruses include, but are not limited to, Norwalk virus, togavirus, flavivirus, reoviruses, papovavirus, hepadnavirus, and hepatitis virus. Examples of retroviruses include avian leukosis-sarcoma, mammalian C-type, B-type viruses, D-type viruses, HTLV-BLV group, lentivirus, and spumavirus (Coffin, J. M., Retroviridae: The viruses and their replication, In Fundamental Virology, Third Edition, B. N. Fields, et al., Eds., Lippincott-Raven Publishers, Philadelphia, 1996).

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells. “Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); simian immunodeficiency virus (SIV), and Maedi-Visna virus (ovine lentivirus).

Methods of using retroviral and lentiviral viral vectors and packaging cells for transducing mammalian host cells with viral particles containing chimeric antigen receptor transgenes are known in the art and have been previous described, for example, in U.S. Pat. No. 8,119,772; Walchli, et al., PLoS One 6:327930, 2011; Zhao, et al., J. Immunol. 174:4415, 2005; Engels, et al., Hum. Gene Ther. 14: 1155, 2003; Frecha, et al., Mol. Ther. 75: 1748, 2010; and Verhoeyen, et al., Methods Mol. Biol. 506:91, 2009. Retroviral and lentiviral vector constructs and expression systems are also commercially available.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing TCR or CAR transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and a-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

In certain embodiments, the nucleic acid encoding a binding proteins or high affinity recombinant TCR specific for a KRAS G12V or Her2-ITD neoantigen may be operatively linked to one or more certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest. In some embodiments, a viral or plasmid vector further includes a transduction marker (e.g., green fluorescent protein, tEGFR, tCD19, tNGFR, etc.).

In certain embodiments, a vector is capable of delivering the a polynucleotide construct to a host cell (e.g., a hematopoietic progenitor cell or a human immune system cell). In specific embodiments, a vector is capable of delivering a construct to human immune system cell, such as, for example, a CD4+ T cell, a CD8+ T cell, a CD4− CD8− double negative T cell, a γδ T cell, a natural killer cell, a dendritic cell, or any combination thereof. In further embodiments, a vector is capable of delivering a construct to a naïve T cell, a central memory T cell, an effector memory T cell, or any combination thereof. In some embodiments, a vector that encodes a construct of the present disclosure may further comprise a polynucleotide that encodes a nuclease that can be used to perform a chromosomal knockout in a host cell (e.g., a CRISPR-Cas endonuclease or another endonuclease as disclosed herein) or that can be used to deliver a therapeutic transgene or portion thereof to a host cell in a gene therapy replacement or gene repair therapy. Alternatively, a nuclease used for a chromosomal knockout or a gene replacement or gene repair therapy can be delivered to a host cell independent of a vector that encodes a construct of this disclosure.

Construction of an expression vector that is used for recombinantly producing a binding protein or high affinity recombinant TCR specific for a KRAS G12V or Her2-ITD peptide antigen can be accomplished by using any suitable molecular biology engineering techniques known in the art, including the use of restriction endonuclease digestion, ligation, transformation, plasmid purification, and DNA sequencing, for example as described in Sambrook, et al. (1989 and 2001 editions; Molecular Cloning: A Laboratory Manual, Cold Spring Harbor Laboratory Press, NY) and Ausubel, et al. (Current Protocols in Molecular Biology (2003)). To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the protein or peptide of interest.

Also provided are host cells that encode (e.g., comprise a heterologous polynucleotide encoding) and/or express a binding protein or high-affinity recombinant TCR as disclosed herein. In some embodiments, the host cell may be a hematopoietic progenitor cell or an immune system cell as disclosed herein, such as a human immune system cell. In any of the presently disclosed embodiments, the immune system cell is a CD4+ T cell, a CD8+ T cell, a CD4− CD8− double negative T cell, a γδ T cell, a natural killer cell, a dendritic cell, or any combination thereof. Additionally, the T cell may be a naïve T cell, a central memory T cell, an effector memory T cell, a stem cell memory T cell, or any combination thereof. In certain embodiments, the host cell is modified to comprise or contain the heterologous polynucleotide using a vector as disclosed herein.

The recombinant host cell may be allogeneic, syngeneic, or autologous (e.g., to a subject that receives the host cell for a therapy). In certain embodiments wherein the host cell encodes an endogenous TCR, the heterologous binding protein or high affinity recombinant TCR expressed by the T cell is capable of more efficiently associating with a CD3 protein as compared to an endogenous TCR. In some embodiments, the binding protein or high affinity recombinant TCR expressed by a host T cell is able to associate with the CD3 complex and shows functional surface expression and immune activity, e.g., production of cytokines and/or killing of antigen-expressing target cells. In certain embodiments, the binding protein or high affinity recombinant TCR may have higher cell surface expression as compared to an endogenous TCR.

In certain embodiments, a recombinant host immune cell according to the present disclosure (e.g., a T cell, a NK cell, a NK-T cell, or the like) that expresses and/or encodes a binding protein or high-affinity TCR specific for a KRAS V12G peptide antigen is capable of producing a cytokine, such as IFN-γ, in the presence of a peptide antigen or a polynucleotide encoding the same, but less so, or not at all, in the presence of a control or reference molecule (e.g., wild-type peptide or polynucleotide encoding the same). In certain embodiments, the recombinant host immune cell is capable of producing IFN-γ when the peptide antigen is present at 10, 1, 0.1, or about 0.01 μg/mL (e.g., when the peptide is introduced to a target cell capable of presenting the peptide antigen and the recombinant host immune cell is in the presence of the target cell). In further embodiments, the recombinant host immune cell is capable of producing at least about 100, 200, 1,000, 2,000, 3,000, 4,000, 5,000, 6,000, 7,000, 8,000, 9,000, or 10,000 μg/mL IFN-γ when in the presence (a) a target cell (e.g., in a 1:2 recombinant host immune cell:target cell ratio) and (b) antigen at from 0.01 μg/mL (or less) to about 100 μg/mL. Cytokine production can be measured using, for example, a cytokine ELISA kit, such as the human IFN-γ ELISA kit from eBioscience, or the ELISpot-Pro kit, from Mabtech.

In certain embodiments, a recombinant host immune cell is capable of producing IFNγ in the presence of a KRAS G12V peptide and an anti-HLA-DQ antibody, an anti-HLA-DR antibody, or both.

In certain embodiments, a recombinant host immune cell is capable of producing IFNγ in the presence of (a) a KRAS G12V peptide antigen and/or a KRAS G12V peptide-encoding nucleic acid (e.g., RNA) and (b) a cell line that (i) expresses HLA-DRB1-1101 or HLA DRB1-1104 and (ii) is capable of presenting a KRAS G12V antigen to the host immune cell.

In certain embodiments, a recombinant host immune cell encodes (i.e., comprises a heterologous polynucleotide encoding) and/or expresses a Her2-ITD-specific binding protein or high affinity recombinant TCR and is capable of producing a cytokine, e.g., IFN-γ, when in the presence of a Her2-ITD peptide antigen or a polynucleotide encoding the same, but produces the cytokine at a lower level when in the presence of a reference Her2 peptide having a wild-type sequence (i.e., the peptide encoded by a wild-type polynucleotide that does not include an Internal Tandem Duplication) or a reference polynucleotide encoding a wild-type Her2 peptide.

In certain embodiments, a recombinant host immune cell is capable of producing at least about 50, 60, 70, 80, or more pg/mL IFN-γ when in the presence of a target (antigen-presenting cell) at a ratio of about 1 recombinant host immune cell:2 target cells and further in the presence of a Her2-ITD peptide antigen at about 0.01 to about 0.05 μg/m L, and/or is capable of producing at least about 100, 500, 1000, 5,000, or 10,000 μg/mL IFN-γ when in the presence of (a) the target cell and (b) the peptide antigen, when the peptide antigen is present at about 0.02, 0.2, 2, or 20 μg/mL, respectively.

In certain embodiments, a host immune cell is capable of producing IFN-γ when in the presence of a target cell, a Her2-ITD peptide antigen or a polynucleotide encoding the same, and an anti-HLA-DR antibody and/or an anti-HLA Class I antibody.

In certain embodiments, a host immune cell is capable of producing IFNγ in the presence of a Her2-ITD peptide antigen and/or a Her2-ITD peptide-encoding RNA and a cell line that expresses HLA-DQB1-0501 or HLA-DQB1-0502 and is capable of presenting the Her2-ITD peptide antigen to the host immune cell.

In any of the presently disclosed embodiments, a host cell, such as a host immune cell, can comprise a chromosomal gene knockout of an endogenous immune cell protein, such as, for example, PD-1, TIM3, LAG3, CTLA4, TIGIT, an HLA component, or a TCR component, or any combination thereof. As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG (SEQ ID NO:159), GIY-YIG (SEQ ID NO:160), HNH, His-Cys box and PD-(D/E)XK (SEQ ID NO:161). Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host immune cell.

In certain embodiments, a binding protein or TCR of interest may be knocked-in to an endogenous TCR locus, thereby knocking-out endogenous TCR and knocking-in the protein of interest. See, e.g., Eyquem et al., Nature 543(7643):113-117 (2017).

In certain embodiments, a host immune cell encoding and/or expressing a binding protein or recombinant high affinity TCR of the present disclosure is capable of preferentially migrating to or localizing in vivo in a target tissue that expresses a cognate antigen (KRAS G12V or Her2-ITD), such as a tumor, but is present at a statistically significant reduced amount in non-adjacent tissue of the same type. By way of illustration, a host immune cell may be present in a lung tumor (e.g., as determined using deep sequencing for the TCR V-region of the encoded binding protein), but is present at a lower level, or not at all, in tissue of the same lung that is not adjacent to the tumor. In some embodiments, non-adjacent tissue comprises or refers to tissue that is removed from a diseased or malignant tissue by at least 3 cm.

In certain embodiments, a host cell is enriched in a composition of cells, such as may be administered to a subject. As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

Also provided herein are unit doses that comprise an effective amount of a modified immune cell or of a composition comprising the modified immune cell. In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4+ T cells, combined with (ii) a composition comprising at least about 30%, at least about 40%, at least about 50%, at least about 60%, at least about 70%, at least about 80%, at least about 85%, at least about 90%, or at least about 95% modified CD8+ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4+ T cells, combined with (ii) a composition comprising at least about 50% modified CD8+ T cells, in about a 1:1 ratio, wherein the unit dose contains a reduced amount or substantially no naïve T cells. In further embodiments, a unit dose comprises (i) a composition comprising at least about 60% modified CD4+ T cells, combined with (ii) a composition comprising at least about 60% modified CD8+ T cells, in about a 1:1 ratio, wherein the unit dose contains a reduced amount or substantially no naïve T cells. In still further embodiments, a unit dose comprises (i) a composition comprising at least about 70% engineered CD4+ T cells, combined with (ii) a composition comprising at least about 70% engineered CD8+ T cells, in about a 1:1 ratio, wherein the unit dose contains a reduced amount or substantially no naïve T cells. In some embodiments, a unit dose comprises (i) a composition comprising at least about 80% modified CD4+ T cells, combined with (ii) a composition comprising at least about 80% modified CD8+ T cells, in about a 1:1 ratio, wherein the unit dose contains a reduced amount or substantially no naïve T cells. In some embodiments, a unit dose comprises (i) a composition comprising at least about 85% modified CD4+ T cells, combined with (ii) a composition comprising at least about 85% modified CD8+ T cells, in about a 1:1 ratio, wherein the unit dose contains a reduced amount or substantially no naïve T cells. In some embodiments, a unit dose comprises (i) a composition comprising at least about 90% modified CD4+ T cells, combined with (ii) a composition comprising at least about 90% modified CD8+ T cells, in about a 1:1 ratio, wherein the unit dose contains a reduced amount or substantially no naïve T cells.

It will be appreciated that a unit dose of the present disclosure may comprise a binding protein, TCR, or recombinant host cell as described herein (i.e., expressing a binding protein specific for a KRAS G12V or HER2-ITD antigen and a modified immune cell expressing a binding protein specific for a different antigen (e.g., a different KRAS or HER2 antigen, or an antigen from a different protein or target, such as, for example, BCMA, BRAF, CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A (e.g., including MAGE-A1, MAGE-A3, and MAGE-A4), mesothelin, NY-ESO-1, PSMA, RANK, ROR1, TNFRSF4, CD40, CD137, TWEAK-R, HLA, tumor- or pathogen-associated peptide bound to HLA, hTERT peptide bound to HLA, tyrosinase peptide bound to HLA, LTβR, LIFRβ, LRP5, MUC1, OSMRβ, TCRα, TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCH1, WT-1, HA1-H, Robo1, α-fetoprotein (AFP), Frizzled, OX40, PRAME, and SSX-2. or the like). For example, a unit dose can comprise modified CD4⁺ T cells expressing a binding protein that specifically binds to a KRAS G12V:HLA or HER2-ITD:HLA complex and modified CD4⁺ T cells (and/or modified CD8+ T cells) expressing a binding protein (e.g., a CAR) that specifically binds to a BRAFV600E antigen.

In any of the embodiments described herein, a unit dose comprises equal, or approximately equal, numbers of engineered CD45RA⁻ CD3⁺ CD8⁺ and modified CD45RA⁻ CD3⁺ CD4⁺ TM cells.

In practicing various embodiments of the present disclosure, standard techniques may be used for recombinant DNA, peptide, and oligonucleotide synthesis; immunoassays; tissue culture; and transformation (e.g., electroporation and lipofection). Enzymatic reactions and purification techniques may be performed according to manufacturer's specifications or as commonly accomplished in the art or as described herein. These and related techniques and procedures may be generally performed according to conventional methods well-known in the art and as described in various general and more specific references in microbiology, molecular biology, biochemistry, molecular genetics, cell biology, virology, and immunology techniques that are cited and discussed throughout the present specification (see, e.g., Sambrook, et al, Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Roitt, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); and Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008)).

### Uses

In another aspect, the present disclosure provides methods treating a subject in need thereof (i.e., having or suspected of having a disease or disorder associated with a KRAS G12V antigen and/or a Her2-ITD antigen by administering to the subject an effective amount of a composition (e.g., binding protein, TCR, recombinant host cell, immunogenic composition, polynucleotide, vector, or related composition) as described herein. Such diseases include various forms of proliferative or hyperproliferative disorders, such as solid cancers and hematological malignancies.

“Treat,” “treatment,” or “ameliorate” refers to medical management of a disease, disorder, or condition of a subject (e.g., a human or non-human mammal, such as a primate, horse, dog, mouse, or rat). In general, an appropriate dose or treatment regimen including a host cell of this disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof.

A “therapeutically effective amount” or “effective amount” of a composition (e.g., binding protein or host cell expressing or encoding the same) of this disclosure refers to that amount of compound or cells sufficient to result in amelioration of one or more symptoms of the disease being treated in a statistically significant manner. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously. A combination may also be a cell expressing more than one active ingredient, such as two different binding proteins that specifically bind to the same or different antigens.

As used herein, “statistically significant” refers to a p-value of 0.050 or less when calculated using the Student's t-test and indicates that it is unlikely that a particular event or result being measured has arisen by chance.

As used herein, the term “adoptive immune therapy” or “adoptive immunotherapy” refers to administration of naturally occurring or genetically engineered, disease-antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre malignant cells, as well as non-neoplastic or non-malignant disorders.

In certain embodiments, a method comprises administering an effective amount of a composition comprising a binding protein, high affinity recombinant TCR, host cell, immunogenic composition, polynucleotide, or vector as described herein to the subject. In certain embodiments, the subject may have or be suspected of having NSCLC, colorectal cancer, pancreas cancer, biliary cancer, breast cancer, ovarian cancer, acute myeloid leukemia (AML) an(other) indication wherein a KRAS G12V neoantigen is a therapeutic target, or an(other) indication wherein a Her2-ITD neoantigen is a therapeutic target. In certain embodiments, the subject (or the subject disease) expresses at least one of a KRAS G12V neoantigen or a Her2-ITD neoantigen.

In general, an appropriate dosage and treatment regimen provides the active molecules or cells in an amount sufficient to provide a benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Also contemplated are pharmaceutical compositions (compositions) that comprise a binding protein, high-affinity recombinant TCR, host (i.e., modified) immune cell, immunogenic composition, polynucleotide, or vector as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

In the case of an adoptive cell therapy, a therapeutically effective dose is an amount of host cells encoding and/or expressing a binding protein or high affinity recombinant TCR specific for a KRAS G12V or Her2-ITD neoantigen) used in adoptive transfer that is capable of producing a clinically desirable result (i.e., a sufficient amount to induce or enhance a specific T cell immune response against cells expressing KRAS G12V or Her2-ITD neoantigens, e.g., a cytotoxic T cell response, in a statistically significant manner) in a treated human or non-human mammal. In various embodiments, the therapeutically effective dose is an amount of CD4+ T cells only. In particular embodiments, T cell is a naïve T cell, a central memory T cell, an effector memory T cell, or any combination thereof.

The amount of cells in a composition or unit dose is at least one cell (for example, one recombinant CD8+ T cell subpopulation (e.g., optionally comprising memory and/or naïve CD8+ T cells); one recombinant CD4+ T cell subpopulation (e.g., optionally comprising memory and/or naïve CD4+ T cells)) or is more typically greater than 10² cells, for example, up to 10⁴, up to 10⁵, up to 10⁶, up to 10⁷, up to 10⁸, up to 10⁹, or more than 10¹⁰ cells. In certain embodiments, the cells are administered in a range from about 10⁴ to about 10¹⁰ cells/m², preferably in a range of about 10⁵ to about 10⁹ cells/m2. In some embodiments, an administered dose comprises up to about 3.3×10⁵ cells/kg. In some embodiments, an administered dose comprises up to about 1×10⁶ cells/kg. In some embodiments, an administered dose comprises up to about 3.3×10⁶ cells/kg. In some embodiments, an administered dose comprises up to about 1×10⁷ cells/kg. In certain embodiments, a recombinant host cell is administered to a subject at a dose comprising up to about 5×10⁴ cells/kg, 5×10⁵ cells/kg, 5×10⁶ cells/kg, or up to about 5×10⁷ cells/kg. In certain embodiments, a recombinant host cell is administered to a subject at a dose comprising at least about 5×10⁴ cells/kg, 5×10⁵ cells/kg, 5×10⁶ cells/kg, or up to about 5×10⁷ cells/kg. The number of cells will depend upon the ultimate use for which the composition is intended as well the type of cells included therein. For example, cells modified to express or encode a binding protein will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. In certain embodiments, a clinically relevant number of cells can be apportioned into multiple infusions that cumulatively equal or exceed 10⁶, 10⁷, 10⁸, 10⁹, 10¹⁰, or 10¹¹ cells. In certain embodiments, a unit dose of the cells can be co-administered with (e.g., simultaneously or contemporaneously with) hematopoietic stem cells from an allogeneic donor. In some embodiments, one or more of the cells comprised in the unit dose is autologous to the subject.

The pharmaceutical compositions described herein may be presented in unit-dose or multi-dose containers, such as sealed ampoules or vials. Such containers may be frozen to preserve the stability of the formulation until infusion into the patient.

As used herein, administration of a composition refers to delivering the same to a subject, regardless of the route or mode of delivery, such as, for example, intravenous, oral vaginal, rectal, subcutaneous, or the like. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., recombinant host cells with one or more cytokines; immunosuppressive therapy such as calcineurin inhibitors, corticosteroids, microtubule inhibitors, low dose of a mycophenolic acid prodrug, or any combination thereof).

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol, or polyethylene glycols in mixtures with water. Aqueous solutions or suspensions may further include one or more buffering agents, such as sodium acetate, sodium citrate, sodium borate, or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired therapeutic effect in association with an appropriate pharmaceutical carrier.

In certain embodiments, a plurality of doses of a composition described herein (e.g., a recombinant host cell) is administered to the subject, which may be administered at intervals between administrations of about two to about four weeks.

Treatment or prevention methods of this disclosure may be administered to a subject as part of a treatment course or regimen, which may comprise additional treatments prior to, or after, administration of the instantly disclosed unit doses, cells, or compositions. For example, in certain embodiments, a subject receiving a unit dose of the (e.g., recombinant host cell is receiving or had previously received a hematopoietic cell transplant (HCT; including myeloablative and non-myeloablative HCT). Techniques and regimens for performing HCT are known in the art and can comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in certain embodiments, a a recombinant host cell of the present disclosure can be administered with or shortly after hematopoietic stem cells in a modified HCT therapy. In some embodiments, the HCT comprises a donor hematopoieitic cell comprising a chromosomal knockout of a gene that encodes an HLA component, a chromosomal knockout of a gene that encodes a TCR component, or both.

The level of a CTL immune response may be determined by any one of numerous immunological methods described herein and routinely practiced in the art. The level of a CTL immune response may be determined prior to and following administration of any one of the herein described KRAS G12V- or Her2-ITD-specific binding proteins or TCRs (or a host cell encoding and/or expressing the same) or immunogenic compositions. Cytotoxicity assays for determining CTL activity may be performed using any one of several techniques and methods routinely practiced in the art (see, e.g., Henkart, et al., “Cytotoxic T-Lymphocytes” in Fundamental Immunology, Paul (ed.) (2003 Lippincott Williams & Wilkins, Philadelphia, Pa.), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a KRAS G12V- or Her2-ITD-derived neoantigen peptide as described herein. A “biological sample” as used herein may be a blood sample (from which serum or plasma may be prepared), biopsy specimen, body fluids (e.g., lung lavage, ascites, mucosal washings, synovial fluid, etc.), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

In some embodiments, the subject receiving the subject composition has previously received lymphodepleting chemotherapy. In further embodiments, the lymphodepleting chemotherapy comprises cyclophosphamide, fludarabine, anti-thymocyte globulin, oxaliplatin, or a combination thereof.

Methods according to this disclosure may further include administering one or more additional agents to treat the disease or disorder in a combination therapy. For example, in certain embodiments, a combination therapy comprises administering a composition (e.g., binding protein, high-affinity recombinant TCR, modified host cell encoding and/or expressing the same, immunogenic composition, polynucleotide, vector) with (concurrently, simultaneously, or sequentially) an immune checkpoint inhibitor. In some embodiments, a combination therapy comprises administering a composition of the present disclosure with an agonist of a stimulatory immune checkpoint agent. In further embodiments, a combination therapy comprises administering a composition of the present disclosure with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVR1G (CD112R), PVRL2, adenosine, A2aR, immunosuppressive cytokines (e.g., IL-10, IL-4, IL-1RA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise a composition of the present disclosure with one or more inhibitor of any one of the following immune suppression components, singly or in any combination.

Accordingly, in certain embodiments, treatment methods according to the present disclosure may further include administering a PD-1 inhibitor to the subject. The PD-1 inhibitor may include nivolumab (OPDIVO®); pembrolizumab (KEYTRUDA®); ipilimumab+nivolumab (YERVOY®+OPDIVO®); cemiplimab; IBI-308; nivolumab+relatlimab; BCD-100; camrelizumab; JS-001; spartalizumab; tislelizumab; AGEN-2034; BGBA-333+tislelizumab; CBT-501; dostarlimab; durvalumab+MEDI-0680; JNJ-3283; pazopanib hydrochloride+pembrolizumab; pidilizumab; REGN-1979+cemiplimab; ABBV-181; ADUS-100+spartalizumab; AK-104; AK-105; AMP-224; BAT-1306; BI-754091; CC-90006; cemiplimab+REGN-3767; CS-1003; GLS-010; LZM-009; MEDI-5752; MGD-013; PF-06801591; Sym-021; tislelizumab+pamiparib; XmAb-20717; AK-112; ALPN-202; AM-0001; an antibody to antagonize PD-1 for Alzheimer's disease; BH-2922; BH-2941; BH-2950; BH-2954; a biologic to antagonize CTLA-4 and PD-1 for solid tumor; a bispecific monoclonal antibody to target PD-1 and LAG-3 for oncology; BLSM-101; CB-201; CB-213; CBT-103; CBT-107; a cellular immunotherapy+PD-1 inhibitor; CX-188; HAB-21; HEISCOIII-003; IKT-202; JTX-4014; MCLA-134; MD-402; mDX-400; MGD-019; a monoclonal antibody to antagonize PDCD1 for oncology; a monoclonal antibody to antagonize PD-1 for oncology; an oncolytic virus to inhibit PD-1 for oncology; OT-2; PD-1 antagonist+ropeginterferon alfa-2b; PEGMP-7; PRS-332; RXI-762; STIA-1110; TSR-075; a vaccine to target HER2 and PD-1 for oncology; a vaccine to target PD-1 for oncology and autoimmune disorders; XmAb-23104; an antisense oligonucleotide to inhibit PD-1 for oncology; AT-16201; a bispecific monoclonal antibody to inhibit PD-1 for oncology; IMM-1802; monoclonal antibodies to antagonize PD-1 and CTLA-4 for solid tumor and hematological tumor; nivolumab biosimilar; a recombinant protein to agonize CD278 and CD28 and antagonize PD-1 for oncology; a recombinant protein to agonize PD-1 for autoimmune disorders and inflammatory disorders; SNA-01; SSI-361; YBL-006; AK-103; JY-034; AUR-012; BGB-108; drug to inhibit PD-1, Gal-9, and TIM-3 for solid tumor; ENUM-244C8; ENUM-388D4; MEDI-0680; monoclonal antibodies to antagonize PD-1 for metastatic melanoma and metastatic lung cancer; a monoclonal antibody to inhibit PD-1 for oncology; monoclonal antibodies to target CTLA-4 and PD-1 for oncology; a monoclonal antibody to antagonize PD-1 for NSCLC; monoclonal antibodies to inhibit PD-1 and TIM-3 for oncology; a monoclonal antibody to inhibit PD-1 for oncology; a recombinant protein to inhibit PD-1 and VEGF-A for hematological malignancies and solid tumor; a small molecule to antagonize PD-1 for oncology; Sym-016; inebilizumab+MEDI-0680; a vaccine to target PDL-1 and IDO for metastatic melanoma; an anti-PD-1 monoclonal antibody+ a cellular immunotherapy for glioblastoma; an antibody to antagonize PD-1 for oncology; monoclonal antibodies to inhibit PD-1/PD-L1 for hematological malignancies and bacterial infections; a monoclonal antibody to inhibit PD-1 for HIV; and/or a small molecule to inhibit PD-1 for solid tumor.

In certain embodiments, a composition of the present disclosure is used in combination with a LAG3 inhibitor, such as LAG525, IMP321, IMP701, 9H12, BMS-986016, or any combination thereof.

In certain embodiments, a composition of the present disclosure is used in combination with an inhibitor of CTLA4. In particular embodiments, a composition is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a composition of the present disclosure is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 20131025779A1.

In certain embodiments, a composition of the present disclosure is used in combination with an inhibitor of CD244.

In certain embodiments, a composition of the present disclosure is used in combination with an inhibitor of BLTA, HVEM, CD160, or any combination thereof. Anti CD-160 antibodies are described in, for example, PCT Publication No. WO 2010/084158.

In certain embodiments, a composition of the present disclosure is used in combination with an inhibitor of TIM3.

In certain embodiments, a composition of the present disclosure is used in combination with an inhibitor of Gal9.

In certain embodiments, a composition of the present disclosure is used in combination with an inhibitor of adenosine signaling, such as a decoy adenosine receptor.

In certain embodiments, a composition of the present disclosure is used in combination with an inhibitor of A2aR.

In certain embodiments, a composition of the present disclosure is used in combination with an inhibitor of KIR, such as lirilumab (BMS-986015).

In certain embodiments, a composition of the present disclosure is used in combination with an inhibitor of an inhibitory cytokine (typically, a cytokine other than TGFβ) or Treg development or activity.

In certain embodiments, a composition of the present disclosure is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a composition of the present disclosure is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, a composition of the present disclosure is used in combination with an inhibitor of VISTA, such as CA-170 (Curis, Lexington, Mass.).

In certain embodiments, a composition of the present disclosure is used in combination with an inhibitor of TIGIT such as, for example, COM902 (Compugen, Toronto, Ontario Canada), an inhibitor of CD155, such as, for example, COM701 (Compugen), or both.

In certain embodiments, a composition of the present disclosure is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a composition of the present disclosure is used in combination with a LAIR1 inhibitor.

In certain embodiments, a composition of the present disclosure n is used in combination with an inhibitor of CEACAM-1, CEACAM-3, CEACAM-5, or any combination thereof.

In certain embodiments, a composition of the present disclosure is used in combination with an agent that increases the activity (i.e., is an agonist) of a stimulatory immune checkpoint molecule. For example a composition can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a composition of the present disclosure with one or more agonist of a stimulatory immune checkpoint molecule, including any of the foregoing, singly or in any combination.

In certain embodiments, a combination therapy comprises a composition of the present disclosure and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a composition of the present disclosure and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer in a subject are well-known to those of ordinary skill in the art.

Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with a composition of the present disclosure. In further embodiments, a cytokine is administered sequentially, provided that the subject was administered the anti-HER2-ITD and/or anti-KRAS G12V composition at least three or four times before cytokine administration. In certain embodiments, the cytokine is administered subcutaneously. In some embodiments, the subject may have received or is further receiving an immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

In certain embodiments, a combination therapy method comprises administering a composition of the present disclosure according to the present disclosure and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Another aspect of the disclosure is directed to a composition (e.g., binding protein, TCR, host cell, polynucleotide, vector, immunogenic composition) of the present disclosure as described herein for use in the treatment of, and/or for use in the preparation of a medicament for treatment of, and/or for use in an adoptive immunotherapy for, any one or more of NSCLC, colorectal cancer, pancreas cancer, AML, biliary tract cancer, breast cancer, ovarian cancer, an(other) indication wherein a KRAS G12V neoantigen is a therapeutic target, or an(other) indication wherein a Her2-ITD neoantigen is a therapeutic target. Certain methods of treatment or prevention contemplated herein include administering a host cell (which may be autologous, allogeneic, or syngeneic) encoding and/or expressing a binding protein or TCR as disclosed herein.

Also provided are methods of treating a subject in need thereof and/or inducing an immune response in a subject, wherein the methods comprises administering an effective amount of an immunogenic composition as described herein to the subject. The subject may be have, or be suspected of having, NSCLC, colorectal cancer, pancreas cancer, ovarian cancer, breast cancer, biliary tract cancer, AML, an(other) indication wherein a KRAS G12V neoantigen is a therapeutic target, or an(other) indication wherein a Her2-ITD neoantigen is a therapeutic target. In some embodiments, the immunogenic composition may be administered two or more times to the subject.

In certain embodiments, the method may further comprise administering an adoptive cell therapy (e.g., as disclosed herein) to the subject. In various embodiments, the method may further comprise administering at least one of an adjuvant or a checkpoint inhibitor to the subject, wherein the adjuvant or the checkpoint inhibitor comprises at least one of IL-2, a PD-1 inhibitor, a PD-L1 inhibitor, or a CTLA-4 inhibitor, or another inhibitor or composition as disclosed herein.

In some embodiments, an immunogenic composition comprises a T cell based neoantigen vaccine may be used (see, e.g., PCT Publication No. WO 2017/192924, of which the T cell vaccines, immunogenicity enhancers, transposon expression constructs, and related methods are incorporated by reference in their entireties entirety). In certain embodiments, an immunogenic composition comprises a liposomal RNA preparation (see, e.g., Kreiter, et al, Nature 520: 692, 2015, which preparations and methods of making the same are incorporated by reference herein in their entireties). In certain embodiments, an immunogenic composition is used to prepare apeptide-pulsed dendritic cell or other antigen-presenting cell, which may be performed ex vivo, in vitro, or in vivo.

The present disclosure also provides a method for preparing antigen-pulsed antigen-presenting cells. In some embodiments, the methods comprise contacting in vitro, under conditions and for a time sufficient for antigen processing and presentation by antigen-presenting cells to take place, (i) a population of antigen-presenting cells that are immunocompatible with a subject, and (ii) a polynucleotide, peptide, immunogenic composition, and/or an expression vector as described herein, thereby obtaining antigen-pulsed antigen-presenting cells capable of eliciting an antigen-specific T-cell response to KRAS G12V or Her2-ITD. The method may further include contacting the antigen-pulsed antigen-presenting cells with one or a plurality of immunocompatible T cells under conditions and for a time sufficient to generate KRAS G12V-specific T cells or Her2-ITD-specific T cells.

Also provided are methods comprising expanding, in vitro or ex vivo, KRAS G12V-specific immune cells or Her2-ITD-specific immune cells as disclosed herein above to obtain one or more clones of the KRAS G12V-specific immune cells or the Her2-ITD-specific immune cells, respectively. In ceratin embodiments, the immune cells comprises T cells and the method comprises expanding the T cells in amounts sufficient for T-cell receptor structural characterization, and determining a T-cell receptor polypeptide encoding nucleic acid sequence for one or more of the one or more clones.

In certain embodiments, the method further comprises transfecting or transducing a population of immune cells in vitro or ex vivo with a polynucleotide comprising the T-cell receptor polypeptide-encoding nucleic acid sequence so-determined, thereby obtaining a population of engineered KRAS G12V-specific immune cells or engineered Her2-ITD-specific immune cells in an amount effective to adoptively transfer or confer an antigen-specific T-cell response to KRAS G12V or Her2-ITD, respectively, when the cells are administered to a subject.

Advances in TCR sequencing have been described (e.g., Robins, et al, 2009 Blood 114:4099; Robins, et al, 2010 Sci. Translat. Med. 2:47ra64, PMID: 20811043; Robins, et al. 2011 (Sep. 10) J. Imm. Meth. Epub ahead of print, PMID: 21945395; and Warren, et al., 2011 Genome Res. 21:790) and may be employed in the course of practicing the embodiments according to the present disclosure. Similarly, methods for transfecting/transducing T cells with desired nucleic acids have been described (e.g., US 2004/0087025) as have adoptive transfer procedures using T cells of desired antigen-specificity (e.g., Schmitt, et al., Hum. Gen. 20: 1240, 2009; Dossett, et al., Mol. Ther. 77:742, 2009; Till et al, Blood 112:2261, 2008; Wang, et al., Hum. Gene Ther. 18:112, 2007; Kuball et al, Blood 109:2331, 2007; US 2011/0243972; US 2011/0189141; and Leen, et al., Ann. Rev. Immunol. 25:243, 2007), such that adaptation of these methodologies to the presently disclosed embodiments is contemplated, based on the teachings herein, including those directed to enhanced affinity TCRs specific for a KRAS G12V (SEQ ID NO:1) or Her2-ITD (SEQ ID NO:22) neoantigen complexed with an HLA receptor.

In some embodiments, immune cell lines may be generated as described by Ho, et al. (see 2006 J Immunol Methods 310 (1-2):40-52)). For example, dendritic cells (DCs) may be derived from a plastic adherent fraction of PBMCs by culture over two days (days −2 to 0) in DC media (CELLGENIX™, Freiburg, Germany) supplemented with GM-CSF (800 U/ml) and IL-4 (1000 U/ml). On day −1, maturation cytokines TNFα (1100 U/ml), IL-113 (2000 U/ml), IL-6 (1000 U/ml) and PGE2 (1 μg/ml) can be added. On day 0, DCs can be harvested, washed, and pulsed with peptide (single peptides at 10 μg/ml or peptide pools at 2 μg/ml) over 2 to 4 hours in serum-free DC media. CD8 T cells can be isolated from PBMCs using anti-CD8 microbeads (MILTENYI BIOTEC™, Auburn, Calif.) and stimulated with DCs at an effector target (E:T) ratio of 1:5 to 1:10 in the presence of IL-21 (30 ng/ml). On day 3, IL-2 (12.5 U/ml), IL-7 (5 ng/ml), and IL-15 (5 ng/ml) can be added. Cells may be restimulated between days 10 and 14 using the plastic adherent faction of irradiated autologous PBMCs as antigen presenting cells (APCs) after being peptide-pulsed for two hours and in the presence of IL-21. After restimulation, cells can be supplemented from day 1 on with IL-2 (25 U/ml), IL-7 (5 ng/ml), and IL-15 (5 ng/ml). T-cell clones can be generated by plating cells at limiting dilution and expanding with TM-LCLs coated with OKT3 (ORTHO BIOTECH™, Bridgewater, N.J.) and allogeneic PBMCs as feeders (REP protocol) as described (see Ho, et al., 2006 J Immunol Methods 310 (1-2):40-52).

### EXAMPLES

The following examples are illustrative of disclosed methods, uses, and compositions. In light of this disclosure, those of skill in the art will recognize that variations of these examples and other examples of the disclosed methods and compositions are possible without undue experimentation.

### Example 1—Clinical Protocol for NSCLC Studies

A single-center study was performed at the Fred Hutchinson Cancer Research Center using NSCLC tissue and non-adjacent lung tissue (as far removed from the malignant lesion as possible, at least 3 cm) obtained after informed consent from four patients (1347, 1490, 1238, and 1139) enrolled on a protocol, including patients undergoing curative intent resections for stage I-III NSCLC approved by the Institutional Review Board. Formalin-fixed, paraffin-embedded tissue from a lymph node resection was obtained from one patient (511) enrolled on a separate protocol approved by the Institutional Review Board. Peripheral blood samples were obtained from patients 511, 1139, and 1238, and leukapheresis products were obtained from patients 1347 and 1490 on protocols approved by the Institutional Review Board. All studies excluded patients with a medical contraindication to blood donation or leukapheresis, and were conducted in accordance with the Belmont Report.

Patient 511 was a 73-year-old woman former smoker who presented at the age of 70 with lung adenocarcinoma metastatic to lymph nodes and bone. She was treated with carboplatin and pemetrexed followed by pemetrexed monotherapy, and was in a period of long term disease stability 3 years after diagnosis when blood donation occurred.

Patient 1347 was a 64-year-old male former smoker who presented with a stage IIB squamous cell carcinoma treated with surgical resection followed by adjuvant carboplatin and paclitaxel. At time of blood donation, he was in surveillance with no evidence of disease.

Patient 1490 was a 62-year-old female former smoker who initially presented with pT2a lung adenocarcinoma that was resected, but subsequently had perihilar and mediastinal local recurrence. She donated blood following initiation of definitive chemoradiation treatment with carboplatin and paclitaxel.

Patient 1139 was a 69-year-old female former smoker who initially had resection of a stage I lung adenocarcinoma had subsequently had local recurrence and brain metastasis treated with stereotactic radiosurgery followed by carboplatin and pemetrexed for 4 cycles followed by pemetrexed maintenance for 6 cycles. Disease progression occurred, and she was treated with nivolumab, and this was followed by disease progression. The patient donated blood while being treated with nivolumab.

Patient 1238 was a 68-year-old nonsmoking man who initially presented with stage IIIA lung adenocarcinoma treated with resection followed by adjuvant pemetrexed and cisplatin, which was followed by progression to metastatic disease 1 year later. The patient was treated with afatanib followed by progression and pembrolizumab followed by progression. The patient then had docetaxel and ramicirumab followed by ramicirumab maintenance, which he was on at the time of his blood donation.

Patient 1490 was a 62-year-old female former smoker who initially presented with pT2a lung adenocarcinoma that was resected, but subsequently had perihilar and mediastinal local recurrence. She donated blood following initiation of definitive chemoradiation treatment with carboplatin and paclitaxel.

TABLE 1
Characteristics of Patients in the Study

| | | | | | | Time | | | | |
|---|---|---|---|---|---|---|---|---|---|---|
| | | | | | | between | | | | |
| | | | | | | resection | | | | |
| | Age at | | | | Stage at | and | | Treatment | | |
| | blood | | Smoking | Stage at | blood | blood | Prior | at blood | | Mutations |
| Pt # | donation | Diagnosis | history | resection | donation | donation | treatment | donation | mSNVs | screened |
| 511 | 73 | Adeno | Yes | IV-lymph | IV | 30 | Carboplatin/ | pemetrexed | 505 | 46 |
| | | carcinoma | | node | | months | pemetrexed | | | |
| 1490 | 62 | Adeno | Yes | IB-Lung | III | 13 | None | Carboplatin, | 130 | 46 |
| | | carcinoma | | tumor | | months | | paclitaxel, | | |
| | | | | | | | | radiation | | |
| 1347 | 64 | Squamous | Yes | IIB-Lung | No | 10 | Carboplatin/ | None | 65 | 57 |
| | | cell | | tumor | evidence | months | Paclitaxel | | | |
| | | Carcinoma | | | of | | | | | |
| | | | | | disease | | | | | |
| 1139 | 69 | Adeno | Yes | Stage IV- | IV | 27 | Carboplatin/ | Nivolumab | 388 | 48 |
| | | carcinoma | | Lung | | months | pemetrexed | | | |
| | | | | tumor | | | | | | |
| 1238 | 68 | Adeno | No | IIIA-Lung | IV | 23 | Cisplatin/ | Ramicirumab | 34+ | 20+ |
| | | carcinoma | | tumor | | months | Pemetrexed, | | Her2 | Her2 |
| | | | | | | | Afatinib, | | ITD | ITD |
| | | | | | | | Pembrolizumab, | | | |
| | | | | | | | Docetaxel/ | | | |
| | | | | | | | ramicirumab | | | |

mSNVs: missense single nucleotide variants.

### Example 2—Nucleic Acid Preparation for Exome Capture and RNA Sequencing

Non-tumor DNA was isolated from non-adjacent lung for patients 1490, 1238, and 1139. Blood was used as non-tumor DNA for patients 511 and 1347. Single cell suspensions derived from tumor, lung tissue, or PBMC from blood were processed with the QIAGEN™ DNA/RNA ALLPREP™ Micro kit to isolate DNA for exome capture, with RNA reserved for subsequent RNA-seq profiling. In addition to DNA isolated from the initial tumor resection, a patient-derived xenograft (PDX) was established from the tumor of patient 1347 and PDX tumor was used for DNA and RNA preparation. Genomic DNA concentration was quantified on an INVITROGEN™ QUBIT® 2.0 Fluorometer (LIFE TECHNOLOGIES-INVITROGEN™, Carlsbad, Calif., USA) and TRINEAN™ DROPSENSE96™ spectrophotometer (CALIPER™ Life Sciences, Hopkinton, Mass.).

### Example 3—Whole Exome Sequencing

Exome sequencing libraries were prepared using the AGILENT™ SURESELECTXT™ Reagent Kit and exon targets isolated using the AGILENT™ All Human Exon v6 (AGILENT™ Technologies, Santa Clara, Calif., USA). 200 ng of genomic DNA was fragmented using a COVARIS® LE220 focused-ultrasonicator (COVARIS®, Inc., Woburn, Mass., USA) and libraries prepared and captured on a SCICLONE® NGSx Workstation (PERKINELMER®, Waltham, Mass., USA). Library size distributions were validated using an AGILENT™ 2200 TAPESTATION™. Additional library QC, blending of pooled indexed libraries, and cluster optimization was performed using LIFE TECHNOLOGIES-INVITROGEN™ QUBIT® 2.0 Fluorometer.

The resulting libraries were sequenced on an ILLUMINA® HISEQ™ 2500 using a paired-end 100 bp (PE100) strategy. Image analysis and base calling was performed using ILLUMINA®'s Real Time Analysis v1.18 software, followed by “demultiplexing” of indexed reads and generation of FASTQ files using ILLUMINA®'s bcl2fastq Conversion Software v1.8.4 (support_illumina_com/downloads/bc12fastq_conversion_software_184_html).

Read pairs passing standard ILLUMINA® quality filters were retained for further analysis, yielding an average of 65.2M read pairs for the tumors and 64.4M read pairs for the normals among samples reported here. Paired reads were aligned to the human genome reference (GRCh37/hg19) with the BWA-MEM short-read aligner (see Li H. arXiv preprint arXiv:13033997. 2013 and Li H, et al. Bioinformatics. 2009; 25(14):1754-60). The resulting alignment files, in standard BAM format, were processed by Picard 2.0.1 and GATK 3.5 (see McKenna A, et al. Genome research. 2010; 20(9):1297-303) for quality score recalibration, indel realignment, and duplicate removal according to recommended best practices (see Van der Auwera G A, et al. Current protocols in bioinformatics. 2013:11.0. 1-.0. 33).

To call somatic mutations from the analysis-ready tumor and normal BAM files, two independent software packages were used: MuTect 1.1.7 (see Cibulskis K, et al. Nature biotechnology. 2013; 31(3):213), Strelka 1.0.14 (see Saunders C T, et al. Bioinformatics. 2012; 28(14):1811-7), and variant calls from both tools, in VCF format, were annotated with Oncotator (see Ramos A H, et al. Human mutation. 2015; 36(4)). Annotated missense somatic variants were combined into a single summary for each sample as follows. First, any mutation annotated as “somatic” but present in dbSNP was removed if it was not also present in COSMIC or its minor allele frequency was greater than 1% (according to the UCSC Genome Browser snp150Common table). Variants supported by both variant callers were retained, and those supported by only one variant caller were subject to manual inspection.

### Example 4—RNA-Seq Data Processing

For patient 1347, direct measurements of RNA expression for candidate mutations were performed using tumor cells from the PDX. An RNA-seq library was prepared from total RNA using the TRUSEQ™ RNA Sample Prep v2 Kit (ILLUMINA®, Inc., San Diego, Calif., USA) and a SCICLONE® NGSx Workstation (PERKINELMER®, Waltham, Mass., USA). Library size distributions were validated using an AGILENT™ 2200 TAPESTATION™ (AGILENT™ Technologies, Santa Clara, Calif., USA). Additional library QC, blending of pooled indexed libraries, and cluster optimization was performed using LIFE TECHNOLOGIES-INVITROGEN™ QUBIT® 2.0 Fluorometer. The library was sequenced on an ILLUMINA® HISEQ™ 2500 to generate 61M read pairs (two 50 nt reads per pair). Reads were first aligned to the mouse reference assembly (mm9) to remove reads from the mouse rather than the engrafted tumor. Remaining reads were aligned to a human RefSeq derived reference transcriptome with RSEM 1.2.19 (see Li B, et al. BMC bioinformatics. 2011; 12(1):323) to derive abundances for each gene in transcript-per-million (TPM) units.

### Example 5—Selection of Mutations for Screening

For each patient, single nucleotide variants (SNVs) were determined by comparison to normal DNA samples and ranked by variant allele frequency and expression to select candidate peptides for screening. For patient 511, mutations called by MuTect 1.1.7 (Cibulskis K, Lawrence M S, Carter S L, Sivachenko A, Jaffe D, Sougnez C, et al. Sensitive detection of somatic point mutations in impure and heterogeneous cancer samples. Nat Biotechnol 2013; 31:213) and Strelka 1.0.14 (Saunders C T, Wong W S, Swamy S, Becq J, Murray L J, Cheetham R K. Strelka: accurate somatic small-variant calling from sequenced tumor—normal sample pairs. Bioinformatics 2012; 28:1811-7) with variant allele frequency greater than 20% were ranked by mean expression in the TCGA for lung adenocarcinoma, and the top 45 mutations were screened.

For patient 1490, all SNVs identified by both MuTect 1.1.7 and Strelka 1.0.14 had variant allele frequencies of 10-40%. These were ranked by mean expression in the TCGA for lung adenocarcinoma, and the top 46 mutations were screened.

For patient 1139, SNVs called by both MuTect 1.1.7 and Strelka 1.0.14 with variant allele frequency of greater than 20% were ranked by mean expression in the TCGA for lung adenocarcinoma, and the top 46 mutations were screened. For patient 1238, <50 mutations were detected, so SNVs called by either MuTect 1.1.7 or Strelka 1.0.14 were ranked by mean expression in the TCGA for lung adenocarcinoma, and SNVs with expression greater than 3 transcripts per million (TPM) were screened.

Somatic variant calling for patient 1347 revealed a large number (>10,000) of C>A/G>T transversions, with low variant allele frequency. A similar number of variants with similar properties were found in the corresponding normal sample as well, suggesting that these were artifacts likely due to oxidation during DNA shearing (Wakabayashi O, Yamazaki K, Oizumi S, Hommura F, Kinoshita I, Ogura S, et al. CD4ρ T cells in cancer stroma, not CD8ρ T cells in cancer cell nests, are associated with favorable prognosis in human nonsmall cell lung cancers. Cancer Sci 2003; 94:1003-9). To avoid this issue with this particular sample, RNA-seq data was leveraged from the corresponding patient-derived xenograft (PDX). The PDX RNA-seq was aligned to the mouse genome (mm9 release of the mouse genome) to suppress reads arising from the mouse. Variant calling on the remaining reads was performed according to the Broad Institute's GATK “Best Practices” RNA-seq variant calling workflow, including two-pass STAR alignment, splitting of spliced reads, and application of the HaplotypeCaller (McKenna A, Hanna M, Banks E, Sivachenko A, Cibulskis K, Kernytsky A, et al. The Genome Analysis Toolkit: a MapReduce framework for analyzing next-generation DNA sequencing data. Genome Res 2010; 20:1297-303) ignoring soft-masked bases (software.broadinstitute.org/gatk/documentation/article.php?id=3891). The HaplotypeCaller also was used to call germline variants in the corresponding normal blood exome sample. Variants found by RNA-seq and not observed in the germline exome capture were retained. To capture additional candidate variants, the MuTect somatic variant caller was also used to compare the analysis-ready PDX RNA-seq BAM file or the PDX exome-capture BAM file against the normal blood exome BAM file. Missense mutations identified through all of the above processes were merged into a set of 235 candidate variants that were all manually inspected with the Integrative Genomics Viewer (IGV)(McKenna et al., (2010)) to retain those supported by the resected tumor exome and the PDX but not observed in the normal blood exome data. Variants were ranked by number of RNA-seq reads supporting the alternate allele at each position, and the top 57 mutations were selected for peptide synthesis. Unlike MuTect 1.1.7, the Strelka variant caller reports candidate somatic insertions and deletions. The fewer than 25 indels reported were manually inspected and subjected to similar filtering criteria as the above point mutations, including variant allele frequency and expected expression of containing gene (aggregated from TCGA LUAD or measured directly from 1347 PDX). Frameshifts likely to cause the resulting protein to be subject to nonsense-mediated decay were also excluded. Apart from the Her2-ITD found in patient 1238, no protein coding indels not predicted to be subject to nonsense mediated decay were identified. Criteria for induction of nonsense-mediated decay is the creation of a stop codon before the terminal exon of the transcript.

### Example 6—T Cell Culture

Peripheral blood mononuclear cells (PBMC) were isolated from blood of patients and normal donors using obtained by density gradient centrifugation using lymphocyte separation medium (Corning), and washed 3-times with PBS supplemented with EDTA (3.6 mM).

Patient PBMCs were stimulated with overlapping 20-mer peptides obtained from ELIM BIOPHARM™. Two peptides spanning each mutation with the mutated residue at position +7 or +13 of the 20 amino acid sequence were used for stimulation, with pools of up to 100 peptides encompassing 50 mutations used for stimulations. Subsequent experiments to analyze T cell reactivity were performed with >80% purity 27-mer peptides with the mutant amino acid at position +13.

Cryopreserved PBMC were thawed and rested overnight in RPMI media with L-glutamine and HEPES (GIBCO™) supplemented with 10% human serum (produced in house), 50 pM beta-mercaptoethanol, penicillin (100 U/mL), and streptomycin (100 U/mL), 4 mM L-glutamine (termed CTL media) and 2 ng/ml recombinant human IL-7 (PEPROTECH®). The following morning, PBMC were washed and 10′ cells were plated in individual wells of a 6 well plate in 5 ml CTL media containing a pool of 1 μg/ml of each peptide without cytokines. Recombinant IL-2 (PEPROTECH®) was added to a final concentration of 10 U/ml on day +3, and half media changes with supplemental IL-2 were performed on days +3, +6, and +9. On day +13, cells from individual wells were harvested and assayed by ELISA and/or cytokine staining assays.

Enrichment of antigen specific T cells identified to be reactive in the initial assay was performed following a stimulation of PBMCs using one or several (as many as 5 pooled) purified mutant peptides and additional cytokines that improved the efficiency of growth with initial stimulation and in subsequent limiting dilution cultures. Briefly, PBMC were first stimulated with 1 μg/ml of the 27-mer mutant peptides in the presence of IL-21 (30 ng/ml), IL-7 (5 ng/ml), IL-15 (1 ng/ml), and IL-2 10 U/ml for 13 days and the cultures were then restimulated with autologous B cells pulsed with 20 μg/ml of a single 27-mer peptide for 5 hours, followed by staining and sorting live cells IFN-γ secreting T cells (Interferon secretion kit APC, Miltenii cat. no. 130-090-762 with included capture and detection reagents), as well as with anti-CD4-pacific blue (clone RPA 14, Biolegend cat. 300521) and anti CD8-FITC (clone HIT8a, BD pharmigen cat. 555634) on a FACSARIA™ II (BD Biosciences).

Sorted T cells included antigen-specific cells, as well as cells that non-specifically produced IFNγ, with unknown purity. In order to isolate clonal or oligoclonal cell populations that were antigen-specific, sorted cells (3 or 10 cells per well) were expanded at limiting dilution in a 96-well plate in the presence of 1.0×105 irradiated allogeneic PBMCs, 2 μg/ml phytohemagglutinin (SIGMA®), and IL-2 (100 U/ml) for 14 to 20 days, with additional IL2 supplemented at day 14. After expansion, T cell lines (10,000-100,000 cells) were incubated with autologous B cells (100,000 cells) pulsed with mutant peptides (10 μg/mL), and IFN-γ production was measured by ELISA to identify those T cells with antigen specificity. Reactive lines were then expanded using a rapid expansion protocol described previously and cryopreserved (see Riddell S R, et al. Journal of immunological methods. 1990; 128(2):189-201). Cryopreserved cells were thawed and rested overnight in CTL media supplemented with 10% DMSO and additional 10% human serum (for a final concentration of 20% human serum (Riddell S R, Greenberg P D. The use of anti-CD3 and anti-CD28 monoclonal antibodies to clone and expand human antigen-specific T cells. J Immunol Methods 1990; 128:189-201). Cryopreserved cells were thawed and rested overnight in CTL media supplemented with IL2 (10 U/mL) prior to assays.

For culture of TILs, 6-12 fragments of patient-derived tumor tissue (2×2×2 mm) were cultured in 24-well plates in T-cell media (RPMI 1640, 10% fetal calf serum, 10 mM HEPES, 100 U/mL Penicillin, 100U/mL Streptomycin, 50 μg/mL gentamicin, 50 pM beta-mercaptoethanol) in the presence of IL2 (6,000 U/mL) for 35 days. TILs were passaged when confluent. Following the conclusion of the 35-day expansion protocol, cells were cryopreserved prior to use in immunological assays.

### Example 7—Antigen Presenting Cells

Autologous B cells were isolated from PBMC using positive selection with magnetic beads coated with antibodies recognizing CD19 (MILTENYI BIOTEC™, cat. 130-050-301) according to the manufacturer's instructions (MILTENYI BIOTEC™). B cells were cultured for seven days in B-cell media comprised of IMDM media (LIFE TECHNOLOGIES™) supplemented with 10% human serum (in-house), 100 U/ml penicillin and 100 μg/ml streptomycin (LIFE TECHNOLOGIES™), 2 mM L-glutamine (LIFE TECHNOLOGIES™), and 200 U/ml IL-4 (PEPROTECH®) in the presence of 3T3 cells expressing human CD40L as described (see Tran E, et al. Science. 2014; 344(6184):641-5). B cells were then restimulated with irradiated (5000 Gy) 3T3 expressing human CD40L cells and fresh medium containing IL-4 was added every three days. B cells were used in assays at day +3 after stimulation. For KRAS-specific T cells, a B-LCL cell line (CLC) was used that is HLA-DRB1-1104 as antigen presenting cells in some experiments. HLA typed LCL cell lines BM14, DEM, LUY, CB6B, and DEU were obtained from the Research Cell Bank (Seattle, Wash.). The remainder of the LCL lines were a gift from Marie Bleakley, Fred Hutchinson Cancer Research Center.

### Example 8—MRNA Expression and Transfection

RNA expression targeted to the endosome was carried out using the method described by the Sahin group (Kreiter S, Selmi A, Diken M, Sebastian M, Osterloh P, Schild H, et al. Increased antigen presentation efficiency by coupling antigens to MHC class I trafficking signals. J Immunol 2008; 180:309-18), where antigens are targeted to the endosome by fusion of the antigen to class I MHC sorting signals.

The mRNA expression construct pJV57 (Veatch J R, Lee S M, Fitzgibbon M, Chow I T, Jesernig B, Schmitt T, et al. Tumor infiltrating BRAFV600E-specific CD4 T cells correlated with complete clinical response in melanoma. J Clin Invest 2018; 128:1563-8) was constructed by gene synthesis (Geneart, Life Sciences), which contained a T7 promoter fused to the N-terminal 25 amino acids of the human HLA-B gene, followed by a BamHI restriction site, the coding sequence of enhanced GFP, an AgeI restriction site, the C terminal 55 amino acids of the human HLA-B gene, followed by the human beta-globin untranslated region followed by a 30-nucleotide poly-A tail and then a SapI restriction site directing cleavage in the poly-A tail.

pJV126 was cloned by ligating the following into Agel/BamHI digested pJV57: annealed oligonucleotides (Ultramers, Integrated DNA Technologies) encoding Her2 amino acids 760-787 flanked by a 5′ AgeI and 3′ BamHI site. pJV127 was made by ligating annealed oligonucleotides (Ultramers, Integrated DNA Technologies) encoding Her2 amino acids 760-787 flanked by a 5′ AgeI and 3′ BamHI site containing the YVMA tandem duplication.

pJV128 and pJV129 were synthesized in an analogous manner, with the first 25 amino acids of KRAS or the first 25 amino acids of KRAS with the G12V substitution, respectively. pJV126 and other plasmids based on JV57 were linearized with SapI (Thermo Fisher), and mRNA was in vitro transcribed using the Highscribe T7 ARCA mRNA kit (New England Biolabs) and purified by lithium precipitation according to the manufacturer's instructions.

For RNA transfection, B cells or B-LCL were harvested, washed 1× with PBS, and then resuspended in Opti-MEM (Life Technologies) at 30×10⁶ cells/mL. IVT RNA (10 mg) was aliquoted to the bottom of a 2-mm gap electroporation cuvette, and 100 mL of APCs were added directly to the cuvette. The final RNA concentration used in electroporations was 100 mg/mL. Electroporations were carried out using a BTX-830 square wave electroporator: 150 V, 20 ms, and 1 pulse. Cells were then transferred to B-cell medium supplemented with IL4 for 16 hours prior to co-cultures (Tran E, Turcotte S, Gros A, Robbins P F, Lu Y C, Dudley M E, et al. Cancer immunotherapy based on mutation-specific CD4ρ T cells in a patient with epithelial cancer. Science 2014; 344:641-5).

### Example 9—Cytokine Release Assays

ELISA assays were performed by incubating 50,000 T cells in 96 well round bottom plates with 100,000 autologous B cells or B-LCL lines pulsed with specific concentrations of peptides in RPMI (GIBCO™) supplemented with 5% heat inactivated fetal bovine serum. IFN-γ in supernatants was diluted 1:1, 1:10, and 1:100 and quantitated using human IFN-γ ELISA kit (EBIOSCIENCE™) in technical duplicate or triplicate. HLA blocking experiments were carried out by adding 20 μg/ml antibody anti class I (BIOLEGEND®, cat. 311411) anti HLA DR (BIOLEGEND® clone L243, cat. 307611) or HLA-DQ (ABCAM™, clone spv-I3, cat. ab23632) to the antigen presenting cells one hour prior to adding peptide. ELISpot assays were performed by incubating 20,000-100,000 T cells with 200,000 autologous B cells pulsed with 20 μg/ml of each peptide in CTL medium using the human IFN-γ ELISPOT-PRO™ kit (MABTECH™) according to the manufacturer's instructions. For intracellular IFN-γ staining, PBMC (100,000) were incubated with autologous B cells (100,000) pulsed with the indicated peptides (20 μg/ml) in the presence of brefeldin A (GOLGIPLUG™, BD BIOSCIENCES™) and then fixed and permeabilized using the BD™ intracellular staining kit (BD BIOSCIENCES™) and analyzed using a FACSCANTO™ II flow cytometer.

### Example 10—TCR Identification and Construction

TCR alpha and beta sequences were obtained from clonal T cell populations by 5′ RACE as described in Examples 11-12. TCR sequences for codon optimized sequences were synthesized and cloned into a lentiviral vector linked by a translational skip sequences as reported previously (see Veatch J R, et al. The Journal of clinical investigation. 2018; 128(4)1563-68). Frequency of TCR Vβ sequences in samples were obtained using the IMMUNOSEQ™ human TCRβ kit from ADAPTIVE BIOTECHNOLOGIES® and analysis on the ADAPTIVE BIOTECHNOLOGIES® software platform.

### Example 11—TCR Vβ and Vα Sequencing

DNA was isolated using the QIAGEN™ DNEASY™ or QIAMP™ micro DNA kits according to the manufacturer's instructions. TCRβ sequencing was performed using the human TCRβ sequencing kit (ADAPTIVE BIOTECHNOLOGIES®) following the manufacturer's instructions and sequenced using a MiSeq (Fred Hutchinson Cancer Research Center Genomics core) with data analysis using ADAPTIVE BIOTECHNOLOGIES® software.

### Example 12—Identification of TCR Sequences

Total RNA was extracted from T cell lines with the RNEASY™ Plus Mini Kit (QIAGEN™). RACE-ready cDNA was generated from RNA using the SMARTER® RACE 5′/3′ Kit (CLONTECH™) according to the manufacturer protocol. CLONEAMP™ HiFi PCR Premix (CLONTECH™) was used to amplify 3′ cDNA fragments. Gene specific primers (Human TCR Cbeta1 Reverse: 5′-CCA CTT CCA GGG CTG CCT TCA GAA ATC-3′ SEQ ID NO:41; Human TCR Cbeta2 Reverse: 5′-TGG GAT GGT TTT GGA GCT AGC CTC TGG-3′ SEQ ID NO:42; Human TCR Calpha Reverse: 5′-CAG CCG CAG CGT CAT GAG CAG ATT A-3′ SEQ ID NO:43) were designed to detect alpha and beta TCR bands (1 Kb). The 3-step touchdown PCR reaction went through 35 cycles of 95° C. for 10 seconds, 60° C. for 15 seconds (decreasing by 0.2° C. each cycle), and 72° C. for 1 minute. The fragments were run on a 1% agarose gel and purified (QIAQUICK™ Gel Extraction Kit, QIAGEN™) for PENTR™ Directional TOPO™ cloning (THERMO FISHER™). DNA was extracted (QIAPREP™ Spin Miniprep Kit, QIAGEN™) from 8-10 clones for each TCR alpha and beta, followed by Sanger sequencing (JV298: 5′-TCG CTT CTG TTC GCG CGC TT-3′ SEQ ID NO:44; JV300: 5′-AAC AGG CAC ACG CTC TTG TC-3′ SEQ ID NO:45).

### Example 13—TCR Vector Construction

TCR construction was in the vector PRRL (see Jones S, et al. Human gene therapy. 2009; 20(6):630-40) further modified by introducing six point mutations into the start codon and putative promoter region of the woodchuck hepatitis virus X protein as described (see Lim C S, et al. RNA biology. 2016; 13(9):743-7), with the TCR β gene preceding the TCR alpha gene separated by a P2A translational skip sequence. Cysteine residues were introduced to facilitate pairing of introduced TCR chains as described (see Kuball J, et al. Blood. 2007; 109(6):2331-8). Specific variable regions and CDR3 sequences are shown in Table 1. Codon optimized DNA fragments containing the TRBV and CDR3 and TRBJ sequences followed by TCRβ sequence with a cysteine substituted at residue 57 followed by a P2A skip sequence and the TRAV and CDR3 sequences followed by TRAJ and TRAC sequences were synthesized as a genestring (LIFE SCIENCES™) and cloned using the NEBUILDER® cloning kit (NEW ENGLAND BIOLABS®) into the lentiviral vector PRRL-SIN linearized with PstI and AscI (THERMO FISHER™) and the sequence verified. The cysteine substituted at residue 57 can ensure pairing of the α-chain and p-chain of the recombinant TCRs and can avoid mispairing with the endogenous TCR α-chain and β-chain. One week after transduction, cells were sorted based on Vβ expression using specific antibodies (Table 2) and expanded as described above. T cells were used in assays or cryopreserved on day 14 after expansion.

TABLE 2
CHARACTERISTICS OF ANTIGEN-SPECIFIC TCR SEQUENCES

| | V- | | J- | |
|---|---|---|---|---|
| Clone | region | CDR3 | region | antibody |
| KRAS | TRBV30 | CAWSALAGARDTQYF | TRBJ2-3 | V beta |
| clone 3 | | (SEQ ID NO: 3) | | 20-FITC |
| beta | | | | (coulter |
| | | | | IM1562) |
| KRAS | TRAV8- | CAVGRSNSGGYQKVTF | TRAJ13 | |
| clone 3 | 3 | (SEQ ID NO: 2) | | |
| alpha | | | | |
| KRAS | TRBV12- | CASSLGLPGTDTQYF | TRBJ2-3 | V beta 8- |
| clone 9 | 4 | (SEQ ID NO: 13) | | PE clone |
| beta | | | | JR.2 (bio- |
| | | | | legend |
| | | | | cat |
| | | | | 348104) |
| KRAS | TRAV8- | CAVTVVNAGNNRKLIW | TRAJ38 | |
| Clone 9 | 1 | (SEQ ID NO: 12) | | |
| alpha | | | | |
| Her2-ITD | TRBV20 | CSAPPLAGDETQYF | TRBJ2-5 | V beta |
| beta | | (SEQ ID NO: 24) | | 2-PE |
| | | | | (mill- |
| | | | | tenyicat |
| | | | | 130-110- |
| | | | | 061) |
| Her2-ITD | TRAV8- | CAVSVNTDKLIF | TRAJ34 | |
| alpha | 6 | (SEQ ID NO: 22) | | |

### Example 14—Crispr-Cas9—Mediated Gene Deletion

CRISPR-Cas9 RNP targeting the first exon of the TCR alpha constant region were created as previously described (Ren J, Liu X, Fang C, Jiang S, June C H, Zhao Y. Multiplex genome editing to generate universal CAR T cells resistant to PD1 inhibition. Clin Cancer Res 2016; 23:2255-66) by mixing equal volumes of 80 μM TracRNA (IDT) with 80 μM of the gRNA AGAGTCTCTCAGCTGGTACA (Kargl J, Busch S E, Yang G H, Kim K H, Hanke M L, Metz H E, et al. Neutrophils dominate the immune cell composition in non-small cell lung cancer. Nat Commun 2017; 8:14381) in duplex buffer (IDT) and heated to 95° C. in a heating block for 5 minutes and allowed to slowly cool. The resulting 40 μM duplexed RNA was the mixed with an equal volume of 24 μM Cas9 protein (IDT) and 1/20 volume of 400 μM Cas9 electroporation enhancer (IDT) and incubated at room temperature for 15 minutes prior to electroporation.

On day 0, CD4+ T cells were isolated from cryopreserved healthy human donor PBMC from 4 patients who provided informed consent on an IRB approved protocol by negative immune selection using the EasySEP human CD4+ isolation kit (StemCell) and stimulated with anti-CD3/anti-CD28 microbeads at a 3:1 bead:cell ratio (Dynabeads, Invitrogen) in the presence of IL2 (50U/mL) and IL7 (5 ng/mL) in CTL media for 2 days. Also on day 0, Lenti-X cells (Clontech) were transiently transfected with the TCR vector, as well as psPAX2 (Addgene plasmid no. 12260) and pMD2.G (Addgene plasmid no. 12259) packaging plasm ids. On day +2, magnetic beads were removed, and 1×10⁶ cells were nucleofected using a Lonza 4D nucleofector in 20 μl of buffer P3 using program EH-115. Cells were allowed to rest for 4 hours in media prior to lentiviral transduction. Lentiviral supernatant was harvested from Lenti-X cells, filtered using 0.45-μm polyethersulfone (PES) syringe filters (Millipore), and 900 μL added to 50,000 activated T cells in a 48-well tissue culture plate. Polybrene (Millipore) was added to a final concentration of 4.4 μg/mL, and cells were centrifuged at 800×g and 32° C. for 90 minutes. Viral supernatant was replaced 16 hours later with fresh CTL supplemented with IL2 (50 IU/mL) and IL7 (5 ng/mL). Half-media changes were then performed every 48-72 hours using CTL supplemented with IL2 and IL7. Transduced T cells were sorted on day+7 or +8 of stimulation using antibodies specific to the transduced TCRVb and grown using a rapid expansion protocol described above for 12-14 days prior to conducting of immune assays.

### Example 15—Statistical Analysis

Statistical analysis was conducted using Graphpad Prism 7.0. Elispot data was analyzed by oneway ANOVA with the Sidak correction for multiple comparisons. Enrichment of TCR Vβ templates within tumor tissue evaluated using the Fisher's exact test.

### Example 16—Results

Tumor specimens were obtained from 4 patients with lung adenocarcinoma and from one patient with squamous cell carcinoma (Table 1). Whole exome sequencing of tumor and normal germline DNA was performed. Protein-coding variants were ranked by variant allele frequency and mRNA expression.

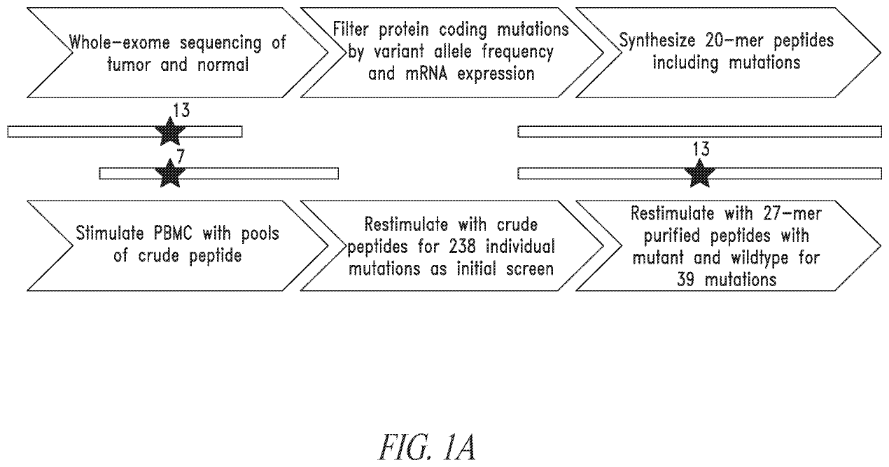

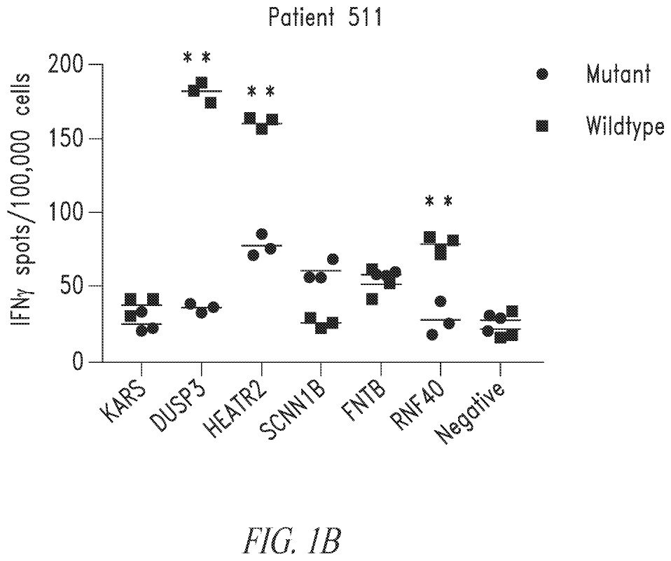

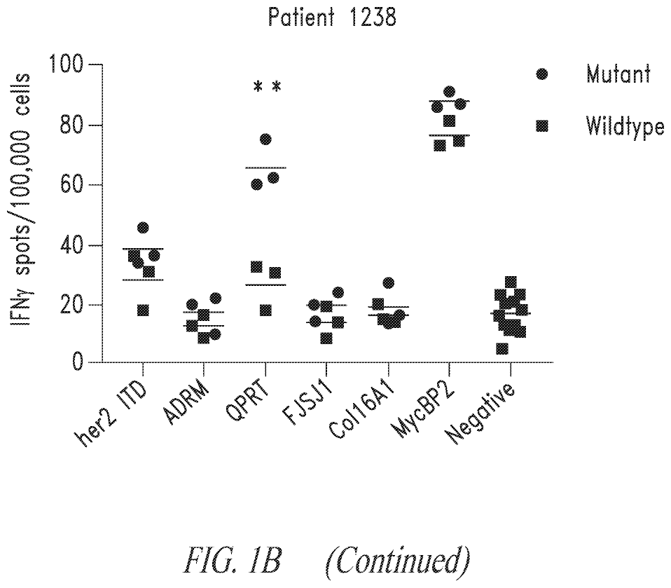

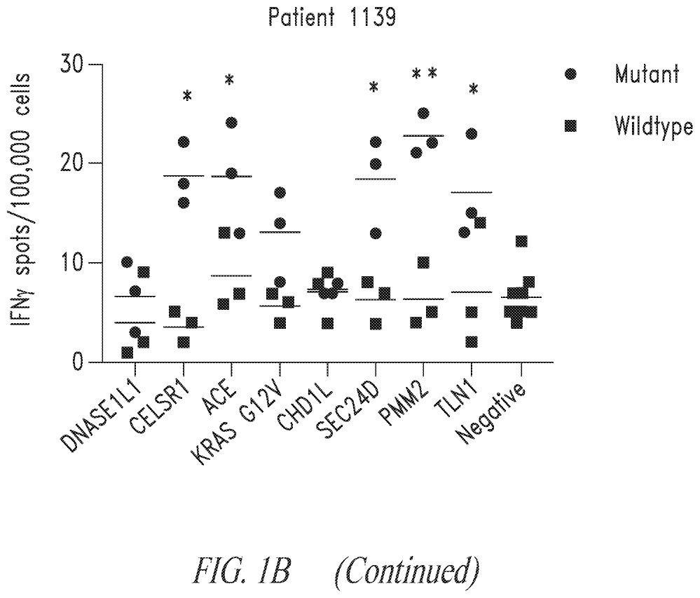

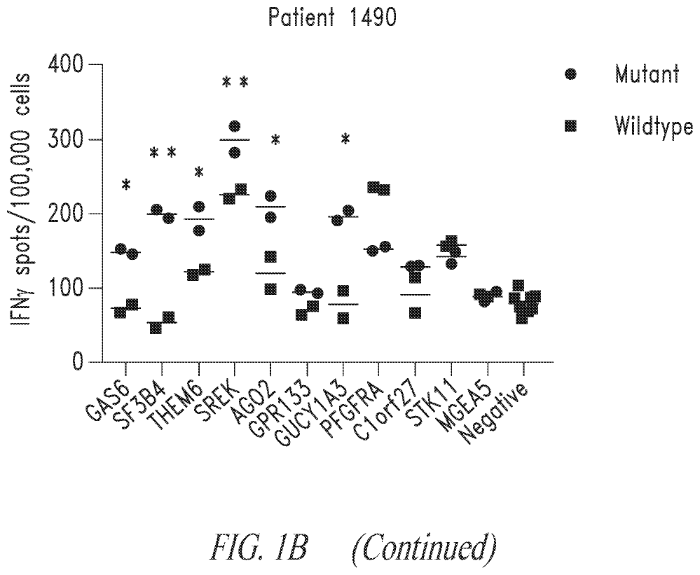

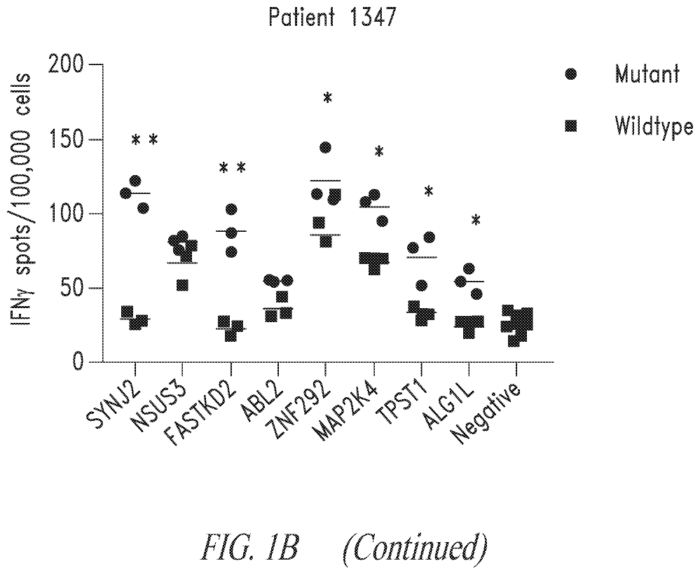

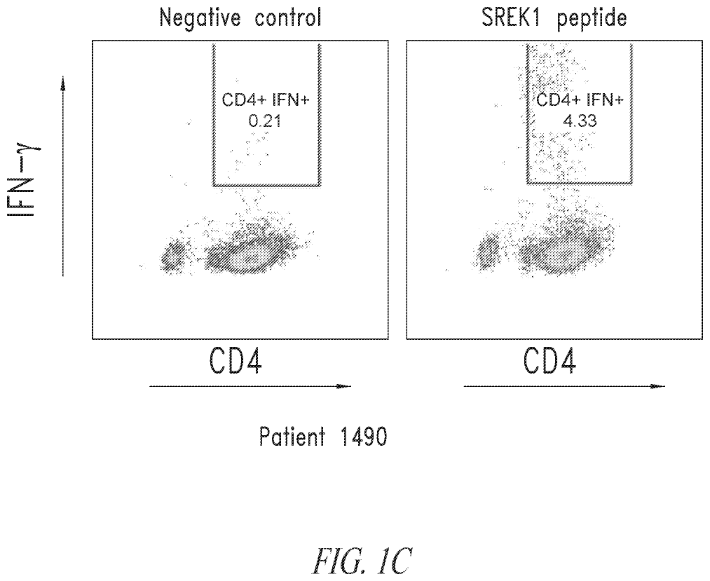

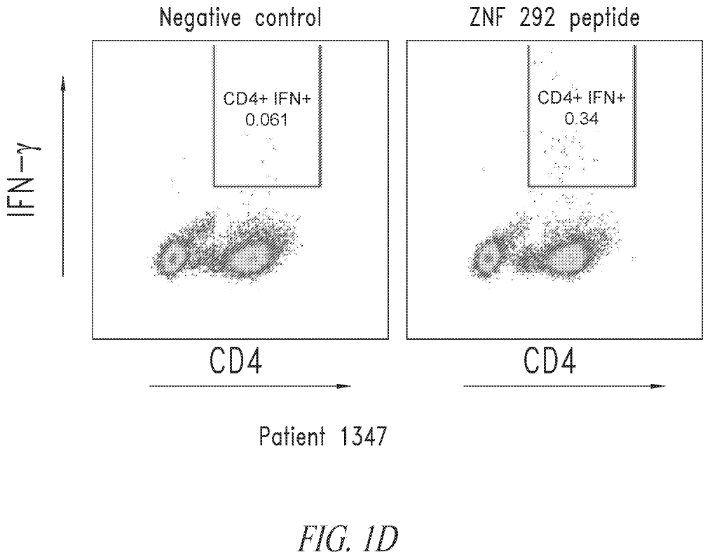

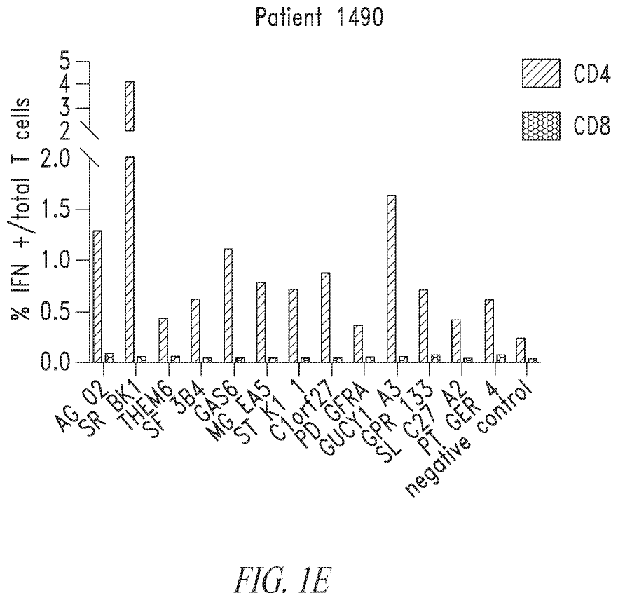

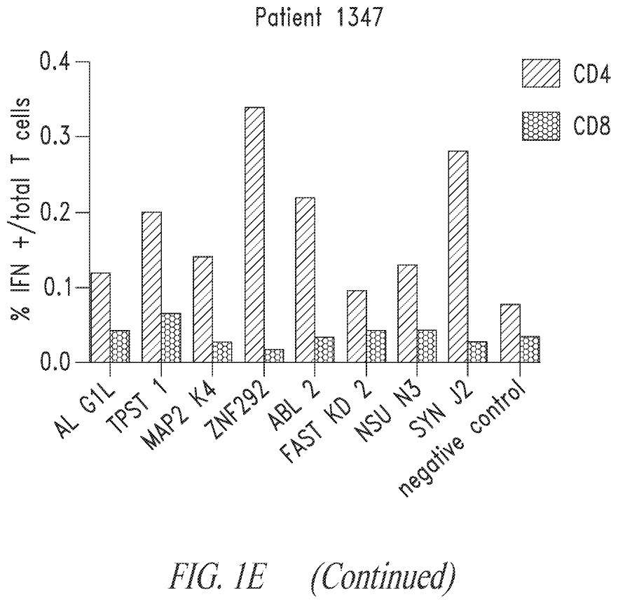

Based on these results and feasibility, 20-57 mutations were selected per patient for analysis of T-cell responses (Table 1; other data not shown). An initial screening assay for T-cell responses to candidate neoantigens was performed by stimulating PBMCs with a pool of overlapping 20-amino acid peptides encompassing each of the mutations and evaluating reactivity by IFNγ Elispot assay (FIG. 1A). T-cell cultures with reactivity above background to a candidate neoantigen were then re-assayed for IFN-γ production in response to purified 27-mer peptides corresponding to the mutant and wild-type sequences (exemplary data shown in FIG. 1B). In total, T-cell responses to 21 of the 238 neoantigens (8.8%) screened were detected and were significantly elevated compared to wild-type peptide responses (p<0.05). Additional weak responses to mutations in KRAS and Her2-ITD were observed and did not meet the cut-off criteria, but were selected for further study because of the important role of these mutations in oncogenesis.

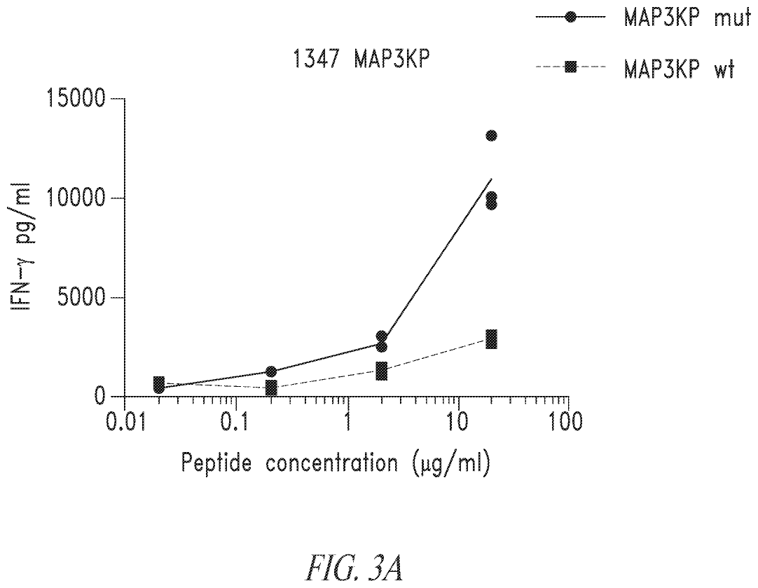

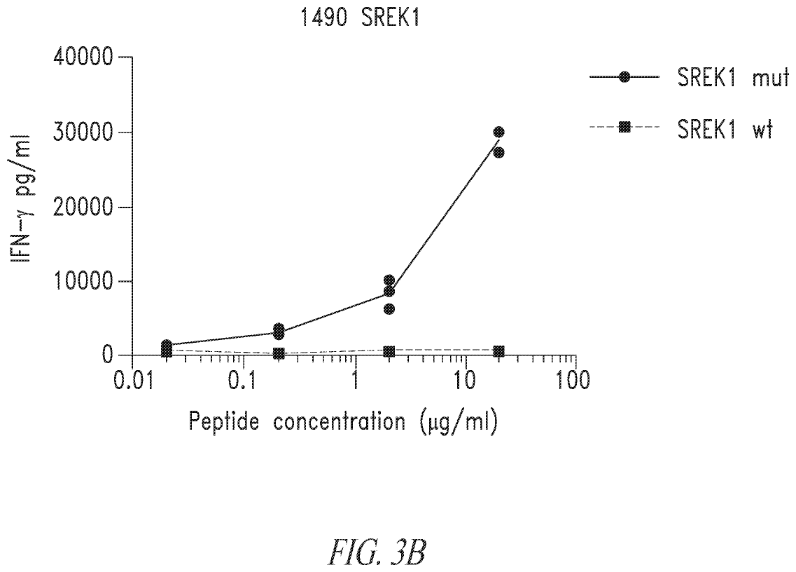

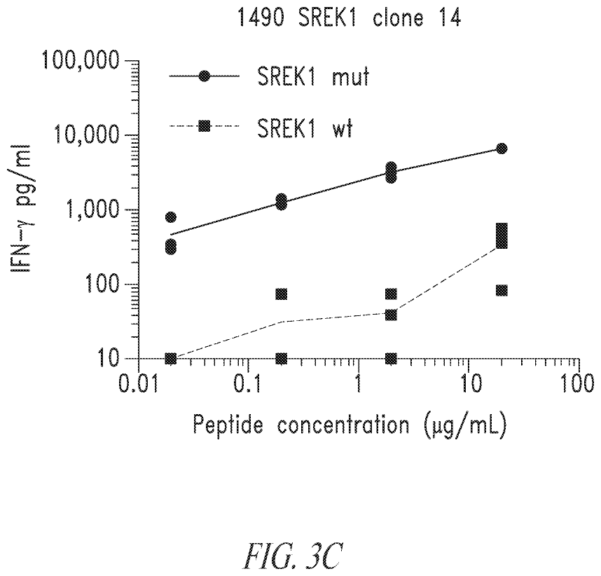

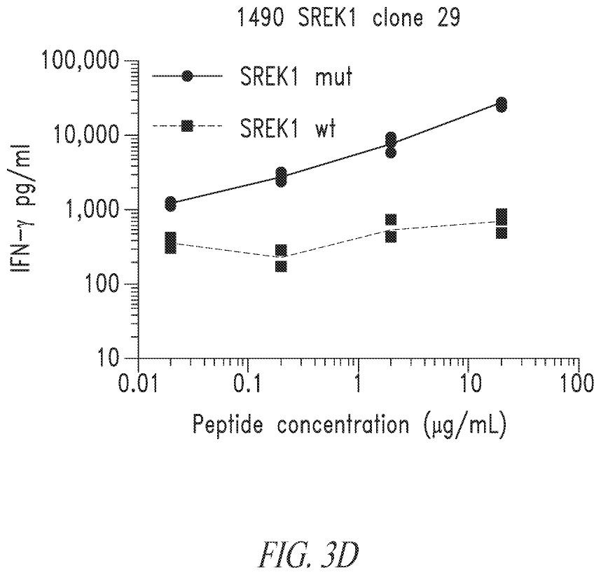

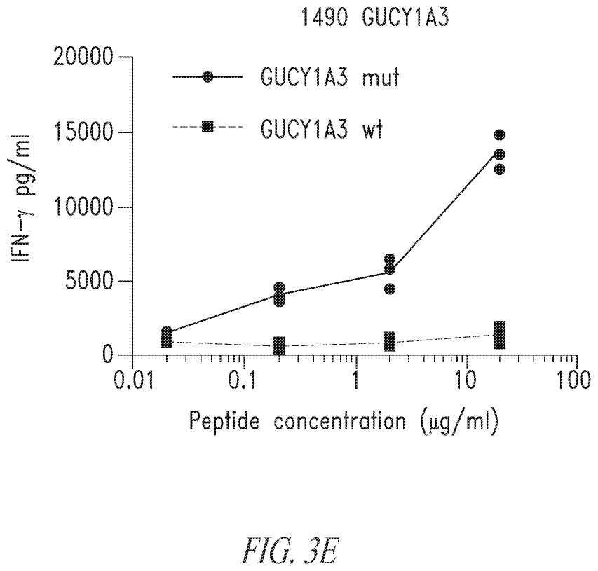

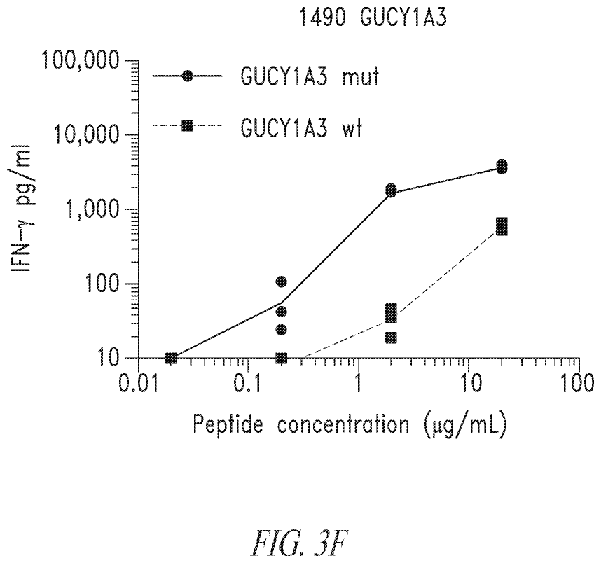

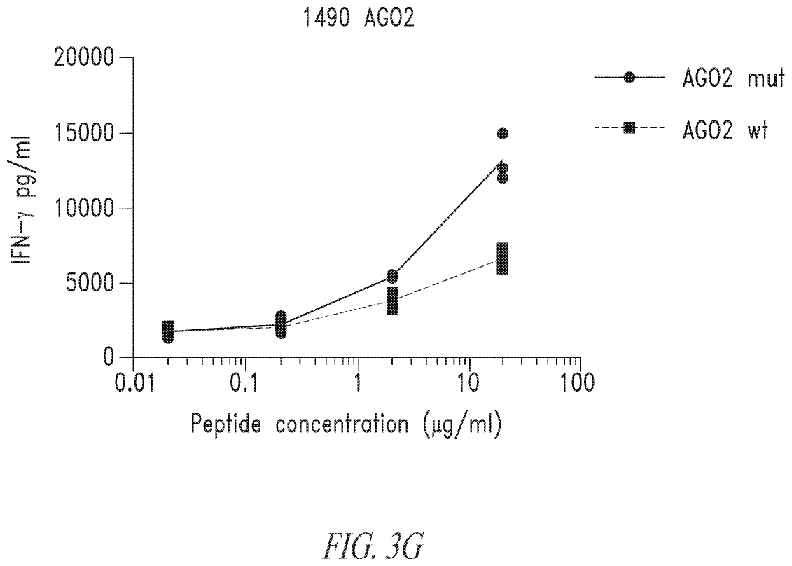

Potential neoantigen-reactive T cells expanded from the blood from patients 1490 and 1347, from whom additional cryopreserved samples and TILs were available, were characterized. PBMCs from these patients were stimulated with purified 27-mer peptides for each of the mutants that elicited a response (meeting the criteria above), and following re-stimulation, IFN-γ+ cells were sorted and expanded by limiting dilution cloning. A single CD4+ clone reactive to the mutation GUCY1A3 was isolated, as well as two different CD4+ clones reactive to a mutation in SREK1 from patient 1490. Each of these clones showed specificity for the mutant relative to the wild-type peptides (FIGS. 3B-3F).

Other isolated T-cell clones were reactive to mutant SREK1 peptide, but the response was similar to that seen with SREK1 wild-type peptide (data not shown), potentially explaining the reactivity to the wild-type peptide observed in the screening Elispot (FIG. 1B). T-cell lines or clones specific for other neoantigens from patients 1490 and 1347 could not be isolated. Two clones specific for SREK1 with different TCRVβ sequences were detected in the initial tumor resection (8/24095 templates), and were enriched relative to the non-adjacent lung tissue from the same resection (1/62424 templates in non-adjacent lung, p=0.0002). The GUCY1A3 TCRVβ was not detected in the tumor resection sample or the lung.

These observations suggested that CD4+ T cells reactive to neoantigens can be isolated from the blood the cells can localize to tumor tissue.

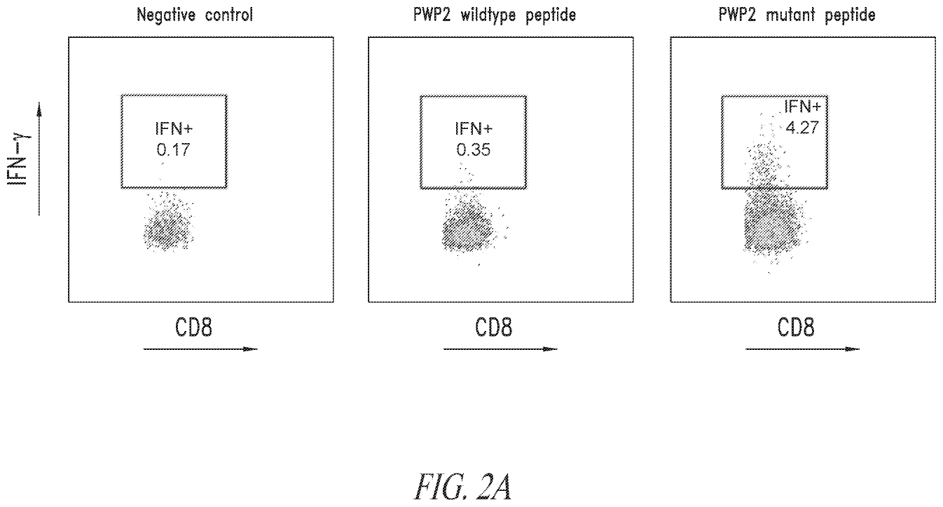

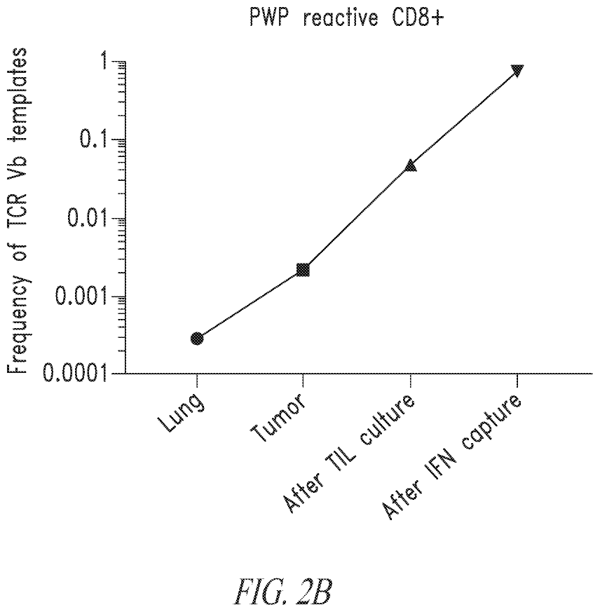

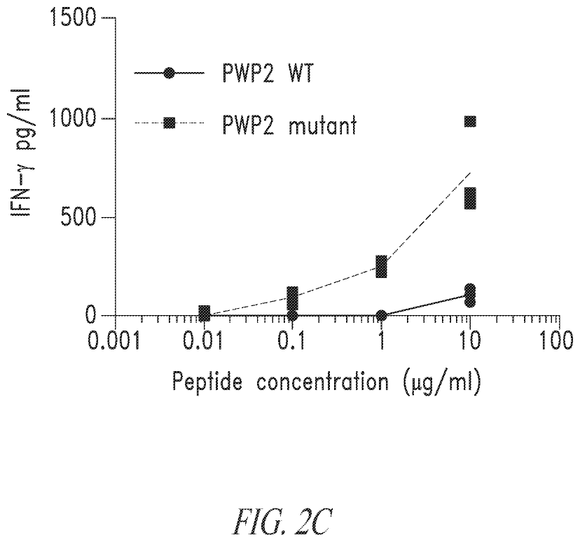

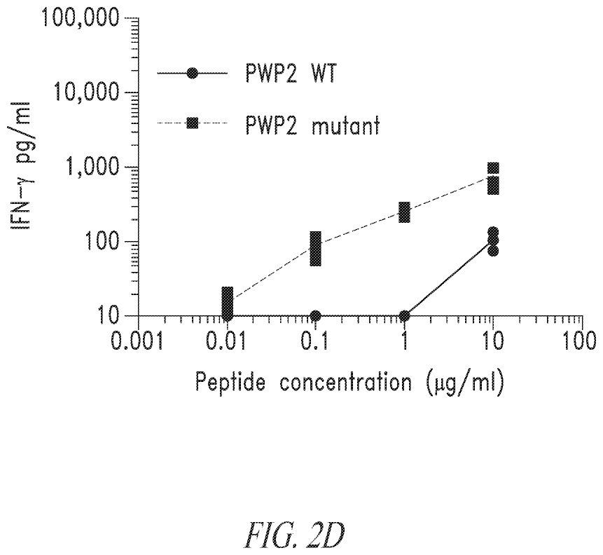

For patients 1490 and 1347, a TIL culture was made from the initial resection sample by culture of tumor fragments in high-dose IL2 (Kargl et al (2017)), and the TILs were assayed for neoantigen reactivity by Elispot and intracellular IFN-γ with 20-mer overlapping peptides described previously. No reactivity was found to screened antigens from patient 1347, but CD8+ T cells in the TILs from patient 1490 were reactive to a mutation in PWP2 (FIG. 2A).

The TCRVβ expressed by sorted PWP2-reactive CD8+ T cells was identified, and the frequency of the PWP2-reactive TCRVβ was determined in the initial tumor resection sample, non-adjacent lung, and after culturing of TILs. The TCRVβ sequence was enriched in the tumor resection relative to the non-adjacent lung (0.2%, 54/24095 templates vs. 0.03%, 18/62424 templates, p<0.0001) and was further enriched by TIL culture (4.8% of templates, FIG. 2B).

A PWP2-reactive T-cell line was expanded from TILs after IFNγ capture and reactivity to the mutant, but not wildtype 10-mer peptide, was confirmed (FIGS. 2C and 2D). TCRVβ sequencing identified the TCRVβ clonotype following stimulation of peripheral blood at a frequency of 0.07% of TCRVβ templates, which may have been too low for detection by the IFN-γ Elispot assay. Thus, T cells with different specificities may be isolated from cultured TIL products and blood, potentially due to the insensitivity of the methods or the difficulty in expanding T cells that may be functionally impaired due to the presence of chronic antigen.

The majority of potential neoantigen-specific T cells identified in blood or tumor by this analysis recognized private, patient-specific mutations consistent with prior studies in other cancers. The relatively weak T-cell responses in the blood to the recurrent driver mutation KRASG12V in patient 1139 and Her2-ITD in patient 1238 did not reach statistical significance, but given the importance of these proteins to the malignant phenotype, were subjected to additional efforts to characterize the specificity. PBMCs from patient 1139 were stimulated twice with KRASG12V peptide, and then identified and sorted IFNγ-secreting CD4+ T cells. T cells were expanded in limiting dilution cultures. Four T-cell cultures were obtained that secreted IFN-γ specifically in response to low concentrations of KRASG12V peptide, but not to the corresponding wild-type KRAS peptide.

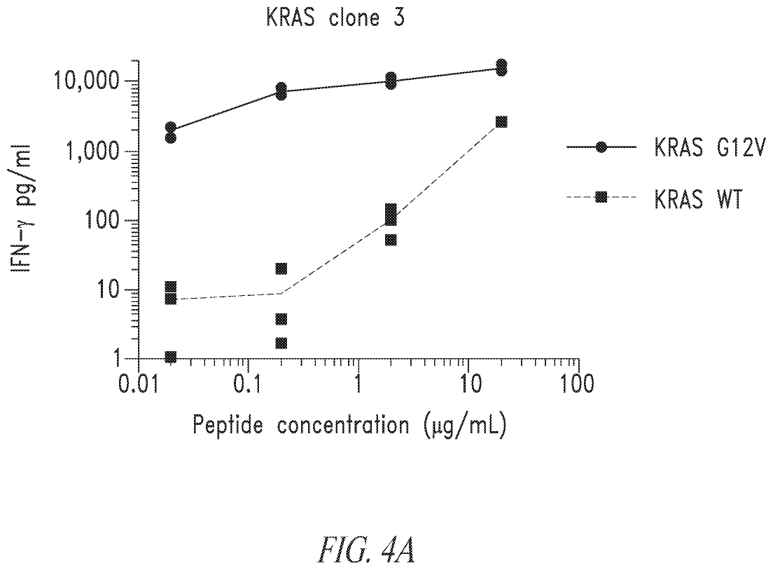

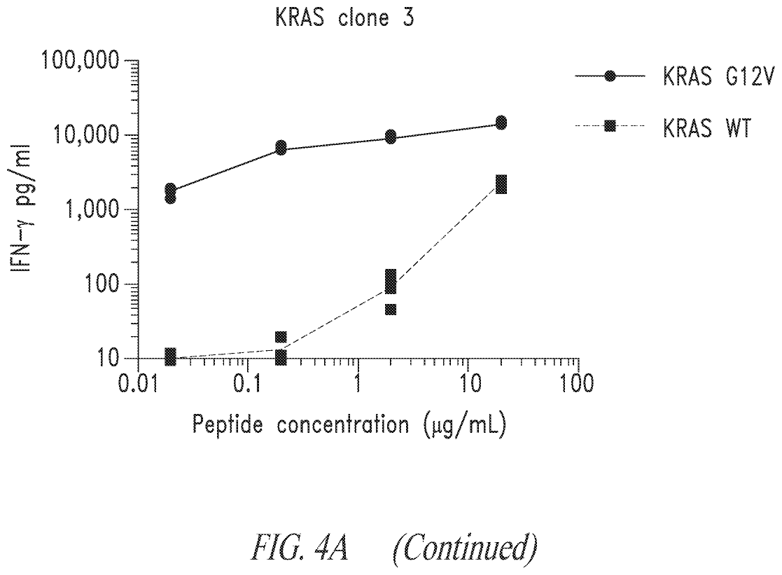

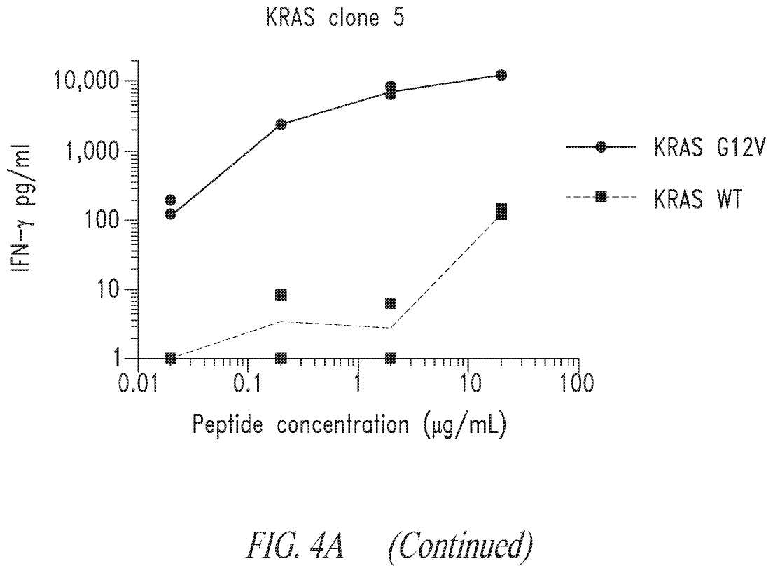

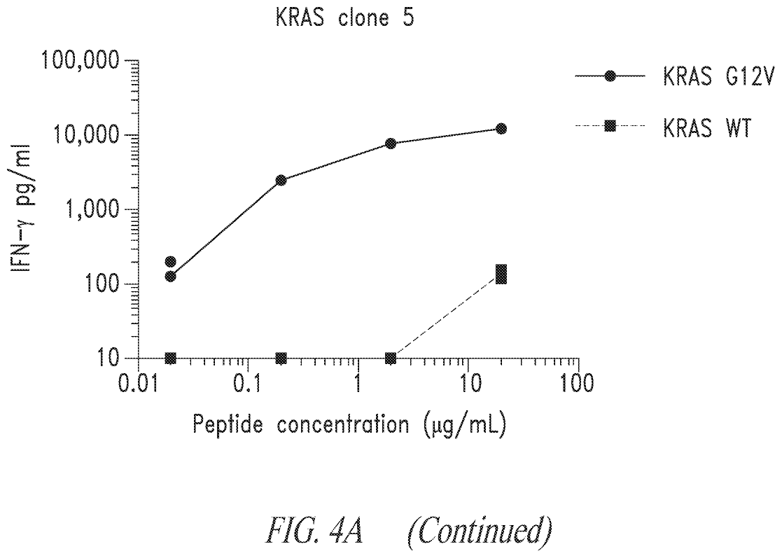

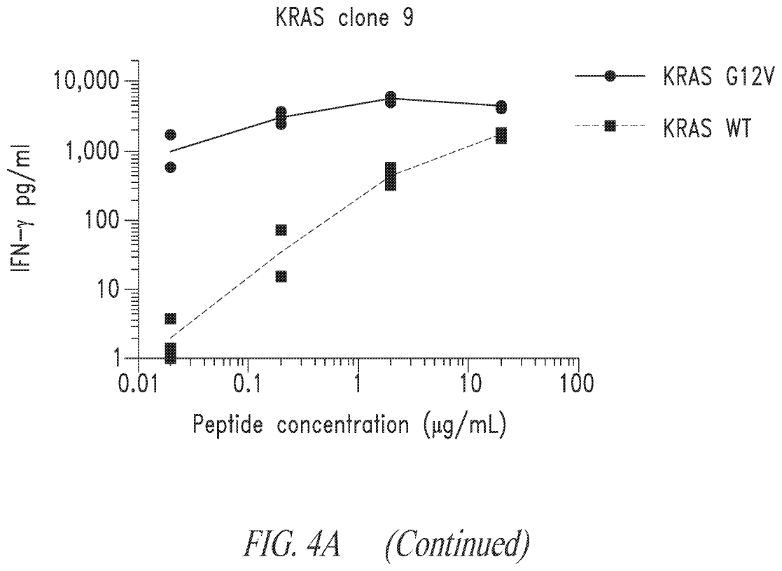

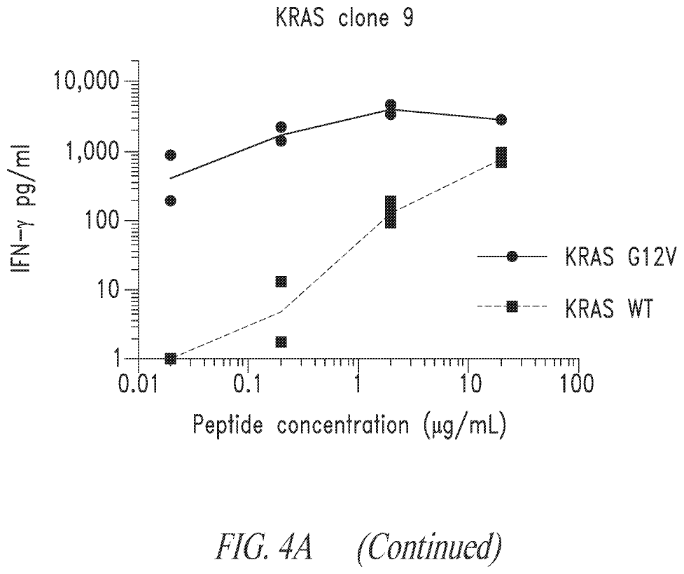

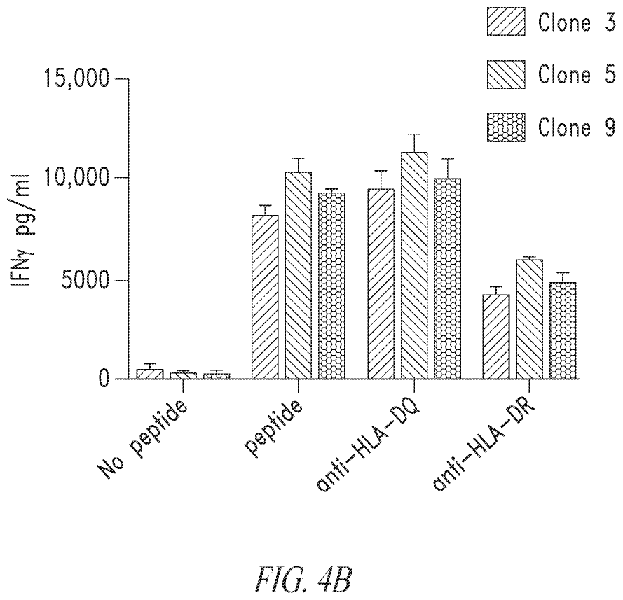

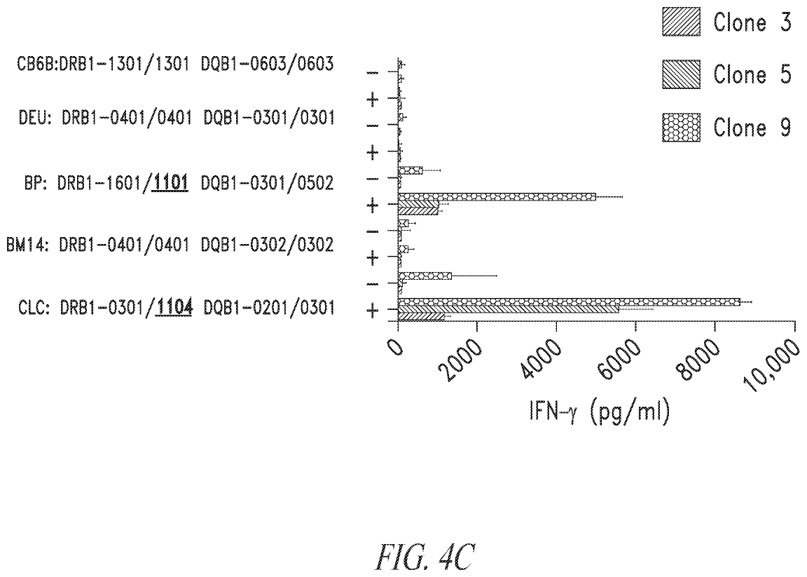

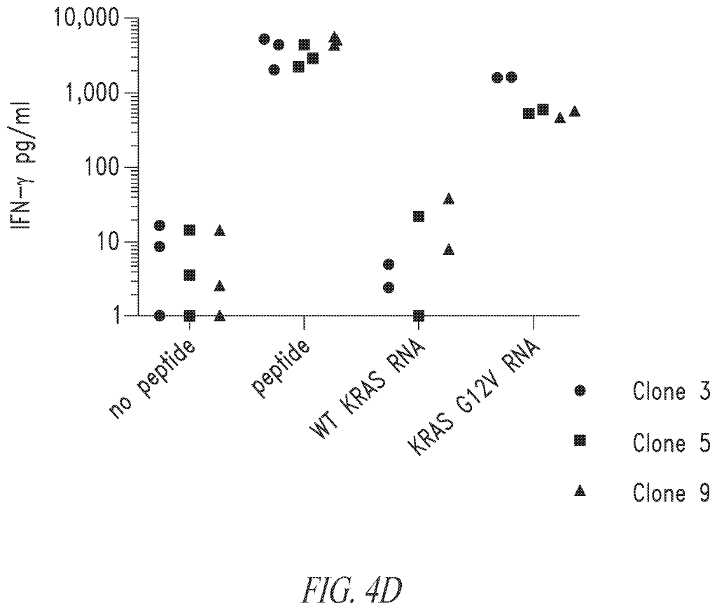

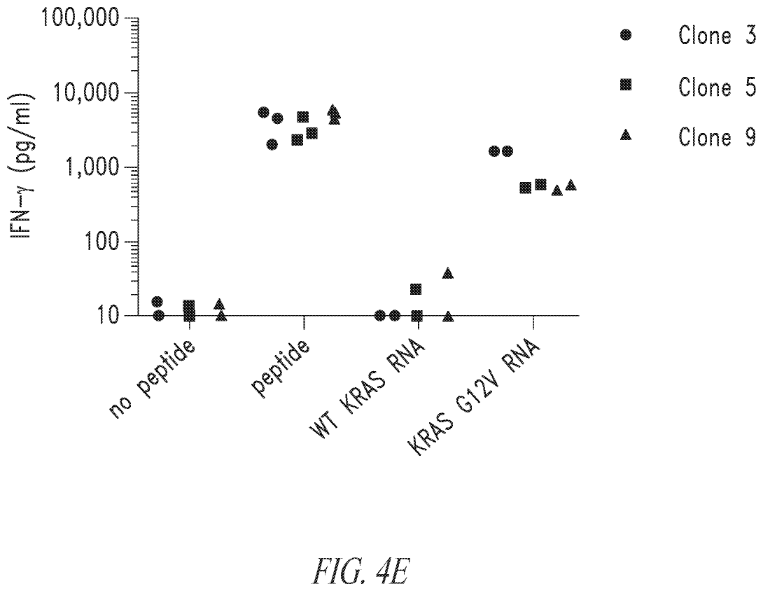

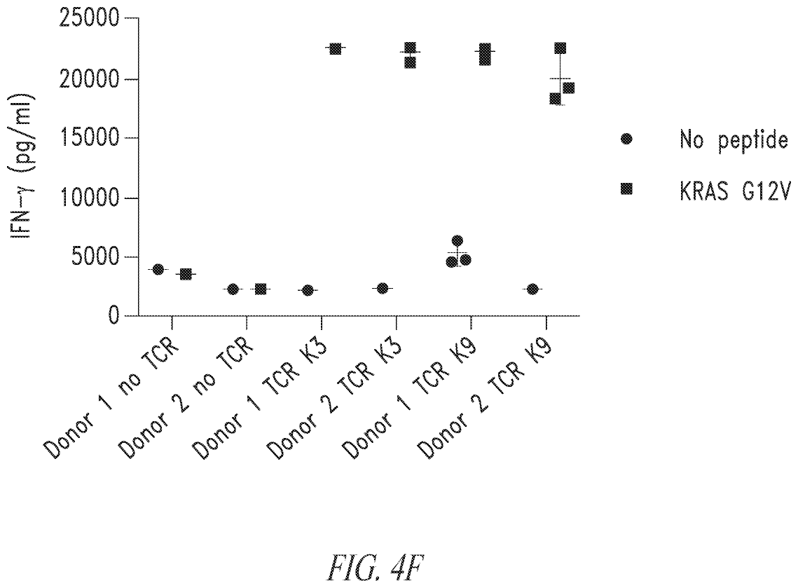

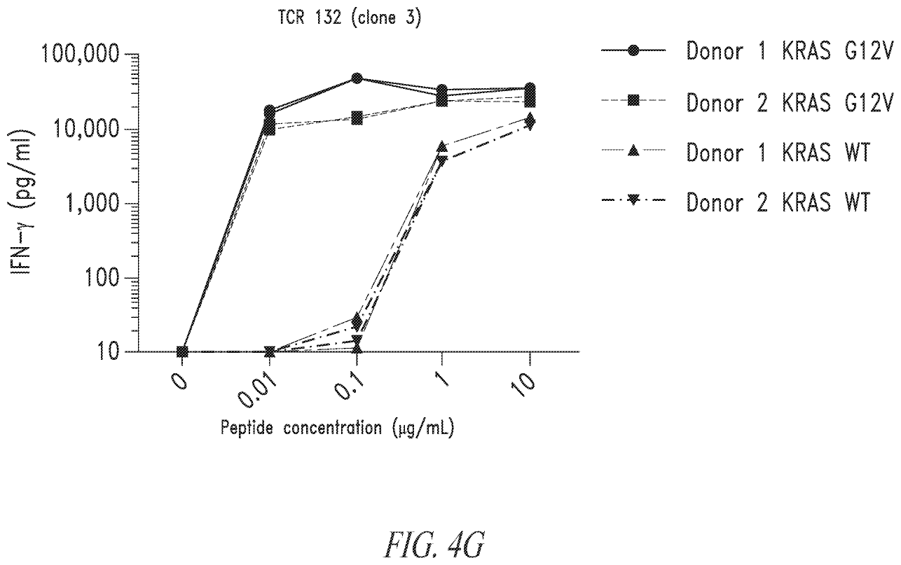

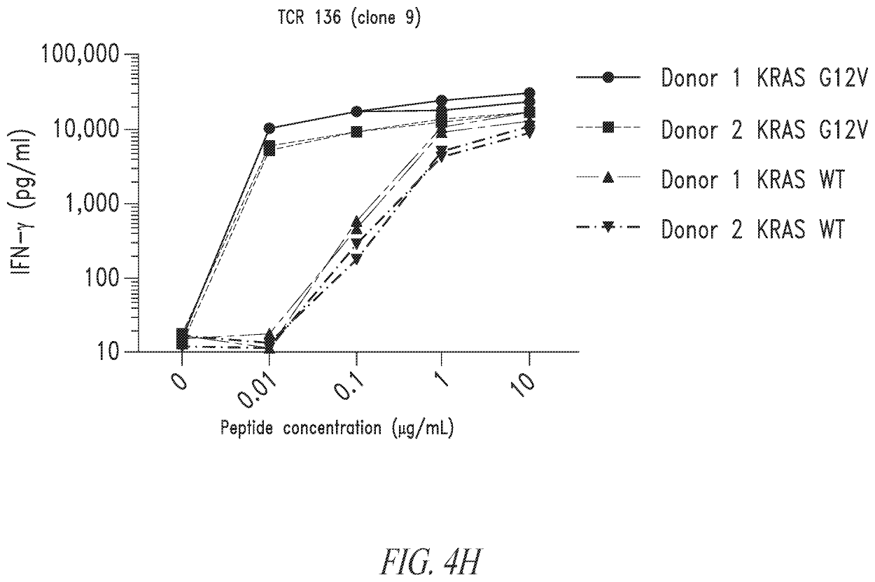

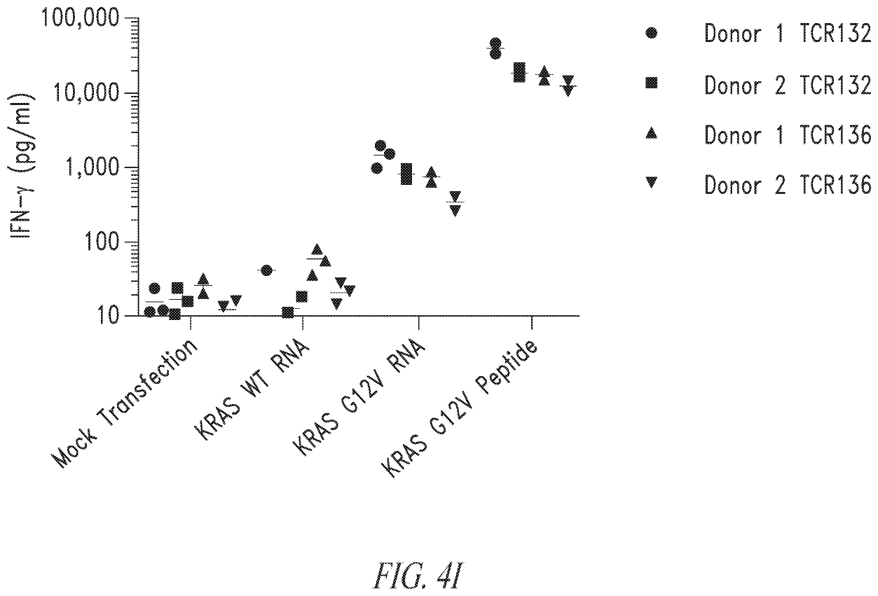

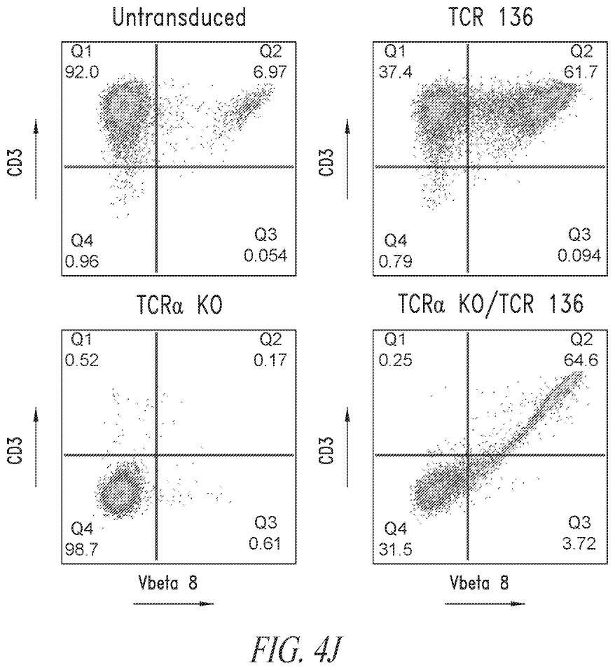

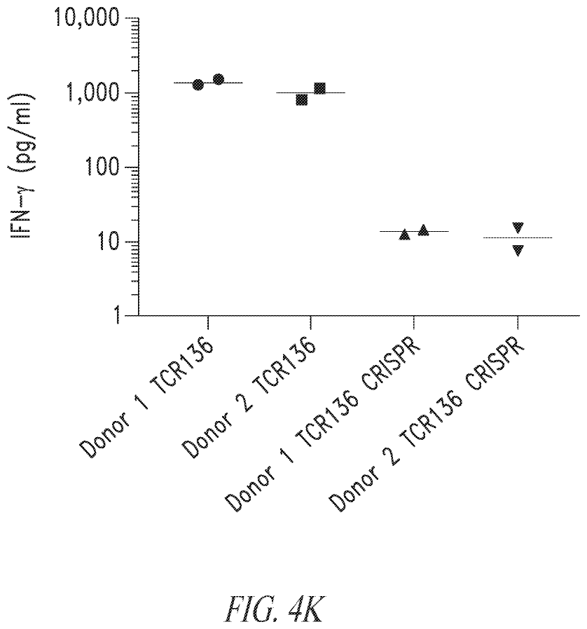

TCRVβ sequencing revealed that these T cells represent monoclonal populations with three distinct TCRVβ clonotypes, referred to as clone 3, 5, and 9 (FIG. 4A). IFN-γ production to KRASG12V was partially blocked by anti-HLA-DR but not anti-HLA-DQ, suggesting restriction by HLA-DR (FIG. 4B). The patient's HLA genotype was HLA-DRB1*11:04/13:01, HLADQB1*03:01/06:03. All three T-cell clones showed reactivity with LCL cell lines expressing HLA-DRB1*11:01 or 11:04 pulsed with KRASG12V peptide, but not peptide-pulsed LCL expressing DQB1*03:01 or DQB1*06:01 in the absence of HLA-DRB1*11, indicating HLA restriction by HLA-DRB1*11 (FIG. 4C). No KRASG12V-specific TCRVβ clonotypes were detected in the resection specimen or non-adjacent lung from the tumor, which were each sequenced to a depth of 10,000 TCRVβ templates.

Reactivity of the KRASG12V-specific T-cell clones to APCs pulsed with wild-type peptide at very high peptide concentrations was observed. Antigens are normally presented to CD4+ T cells after endogenous processing in the endosome (Kreiter et al. (2017)). Thus, to determine whether the KRASG12V-reactive T-cell clones recognized processed antigen, HLA-matched B-LCLs were transfected with minigene constructs encoding either KRASG12V or wild-type KRAS with endosomal targeting sequences. Each of the three clones specifically recognized cells expressing KRASG12V but not wild-type KRAS sequences (FIGS. 4D, 4E), indicating specificity for endogenously processed neoantigen. KRASG12V-specific TCRVβ and Vα sequences from T-cell clones were obtained by 5′ RACE, lentiviral vectors encoding these TCRs were constructed. Transduction of the TCRs from clones 3 and 9 into CD4+ T cells from two normal donors conferred specificity for target cells pulsed with peptides or those expressing KRASG12V but not wild-type KRAS sequences (FIGS. 4F-4I).

In these experiments, donor T cells underwent CRISPR-Cas9-mediated disruption of exon 1 of the endogenous TCRα constant region gene (TRAC) prior to gene transfer of the transgenic TCR (FIG. 4J) to minimize background activation of these cells with allogeneic antigen presenting cells (FIG. 4K). T cells engineered with the KRASG12V-specific TCRs exhibited recognition of target cells pulsed with low concentrations of mutant peptide that were >2 log 10 lower than the wild-type KRAS peptide.

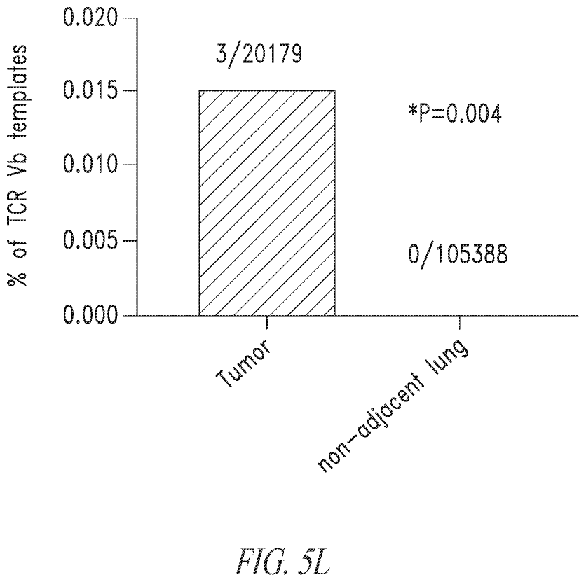

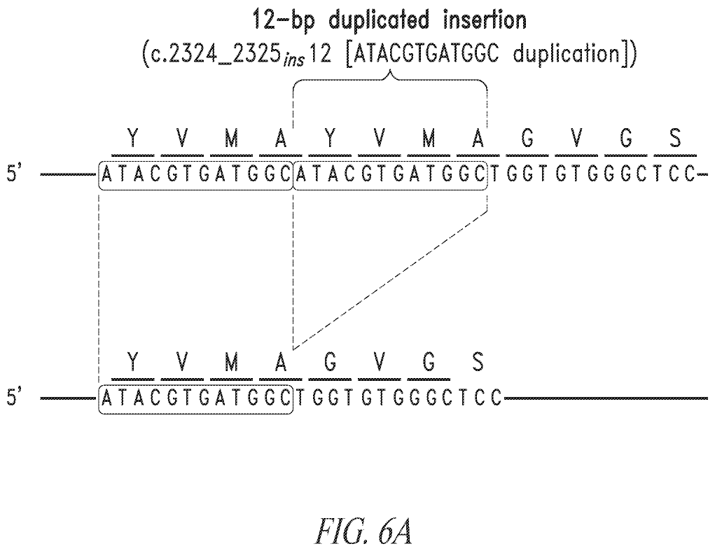

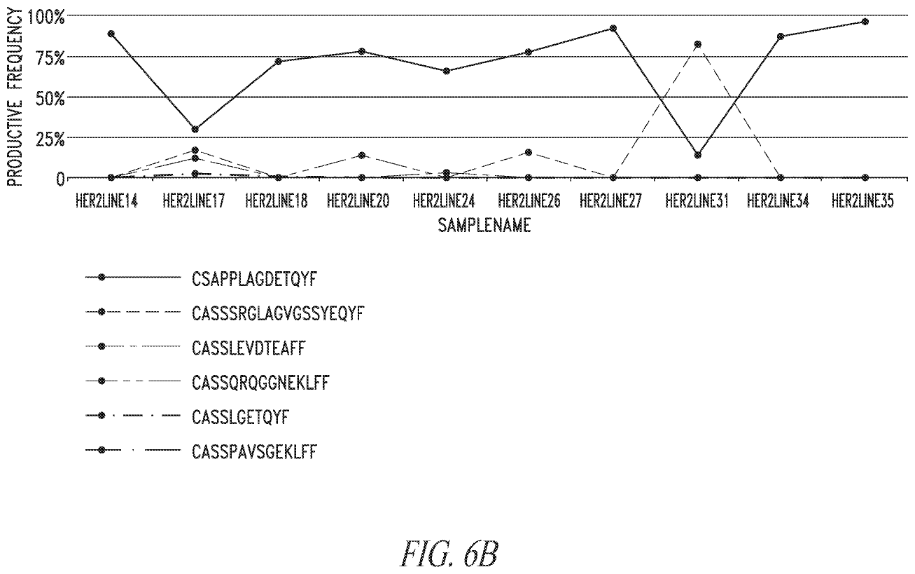

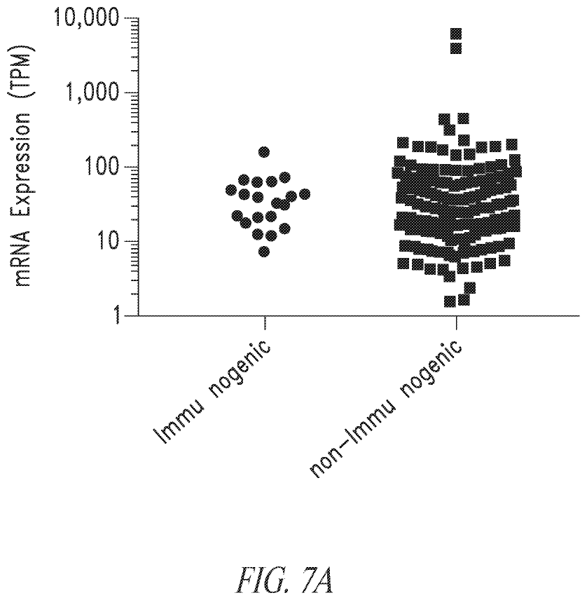

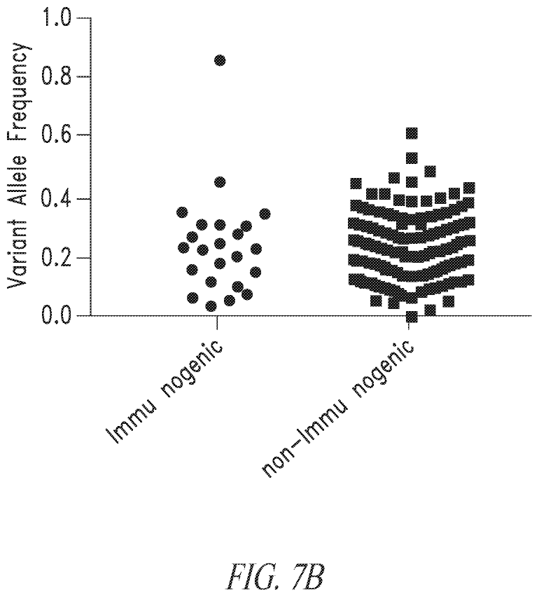

Patient 1238 exhibited a weak CD4+ T-cell response to the recurrent Her2 exon 20 insertion that creates an in-frame duplication of the amino acids YVMA (Her2-ITD) (FIGS. 6A and 1B). The same approach as described above to isolate KRASG12V-specific T cells was successfully used isolated Her2-ITD—specific CD4+ T-cell lines.

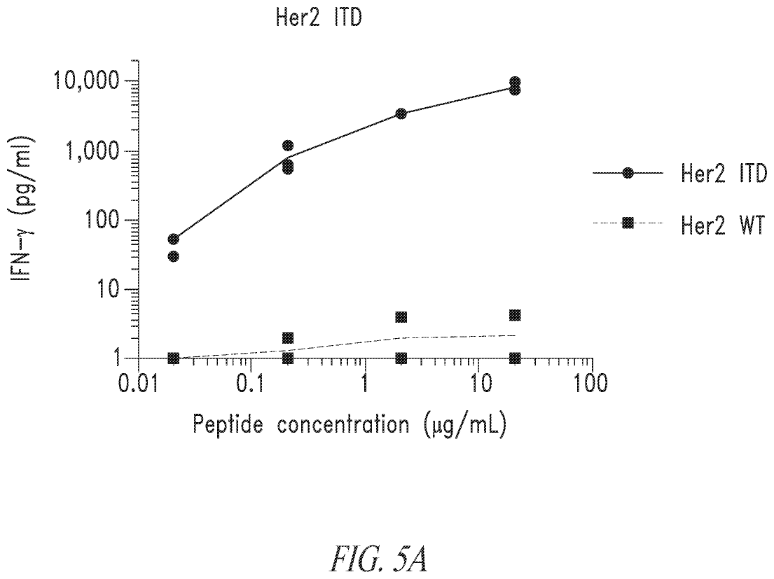

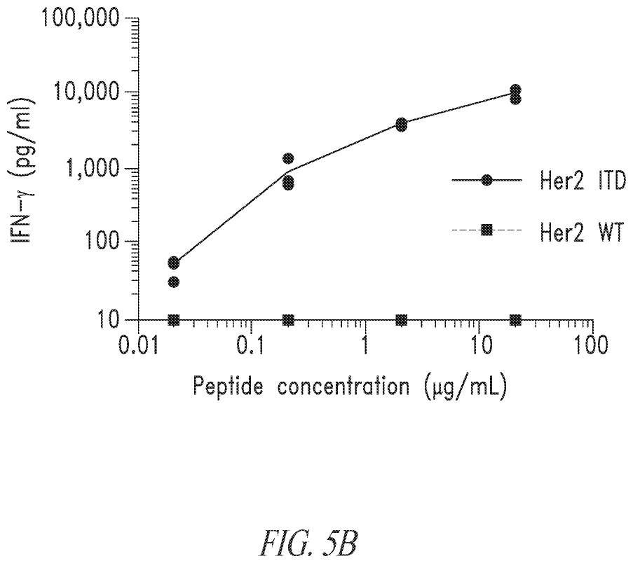

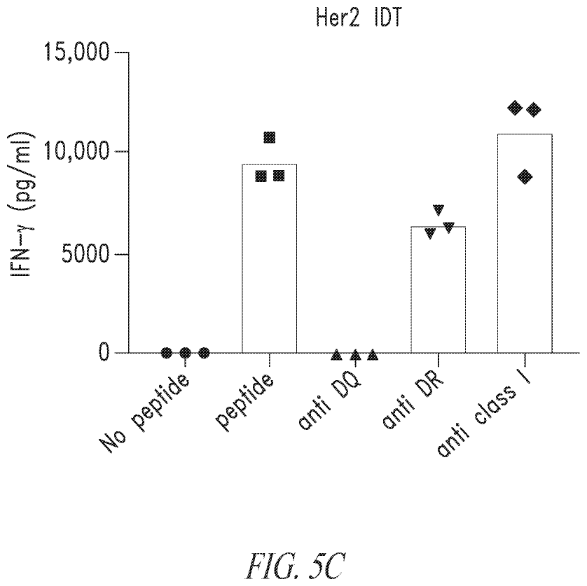

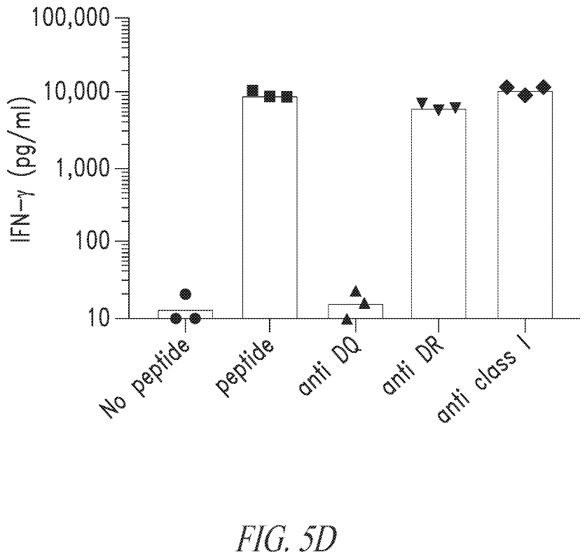

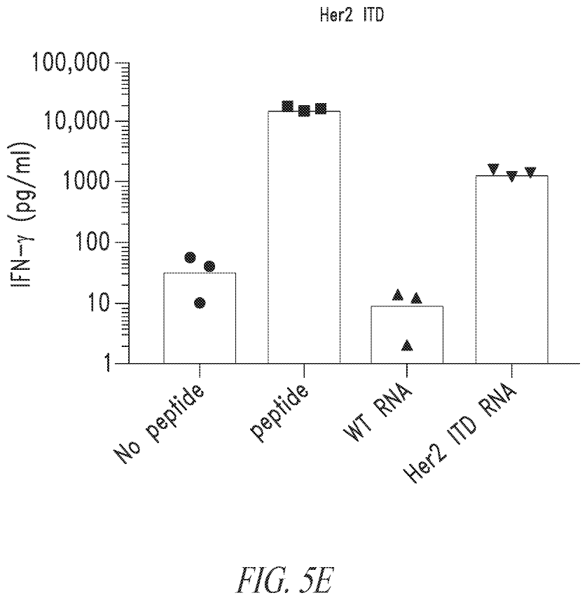

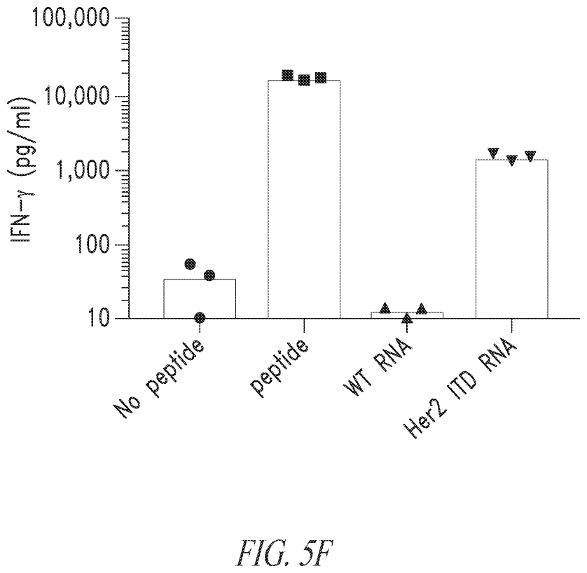

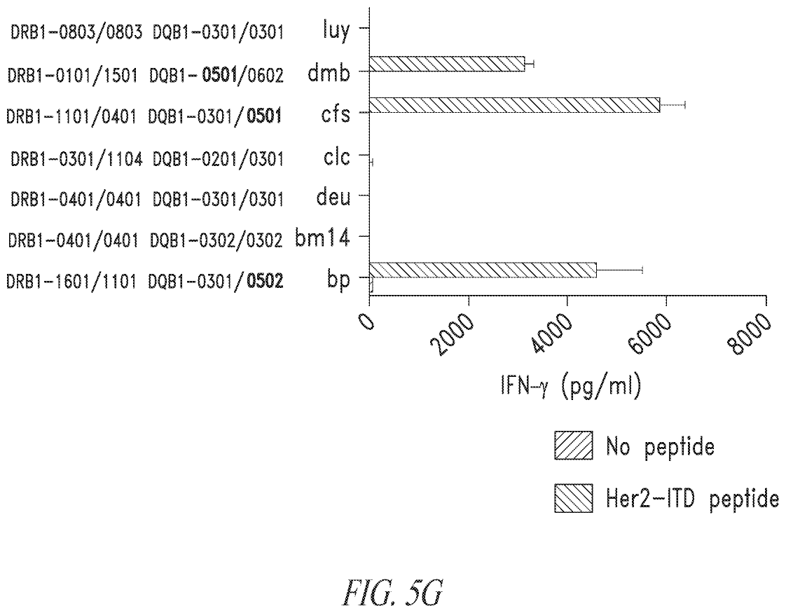

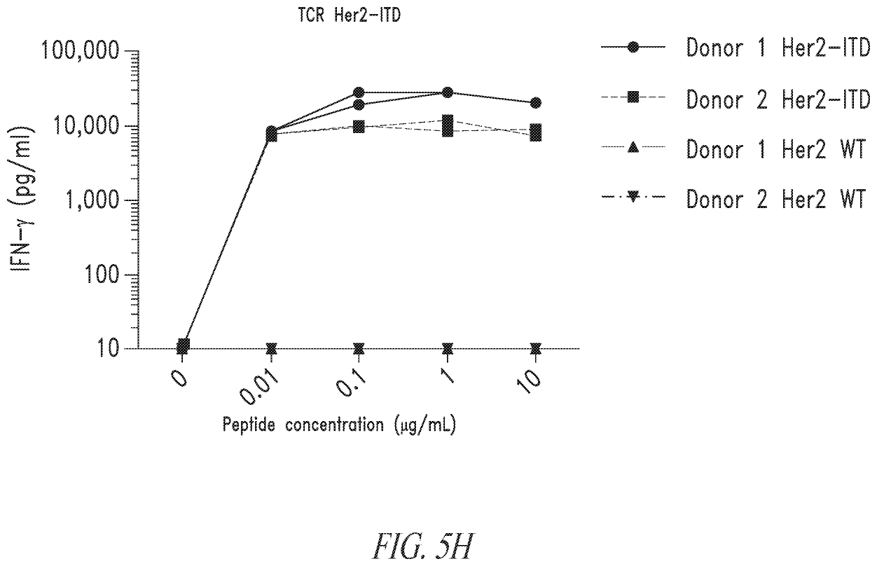

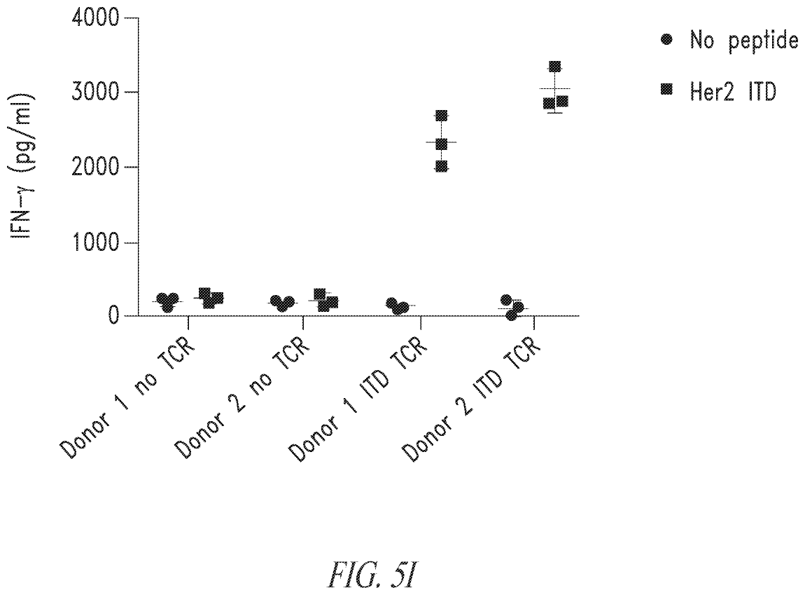

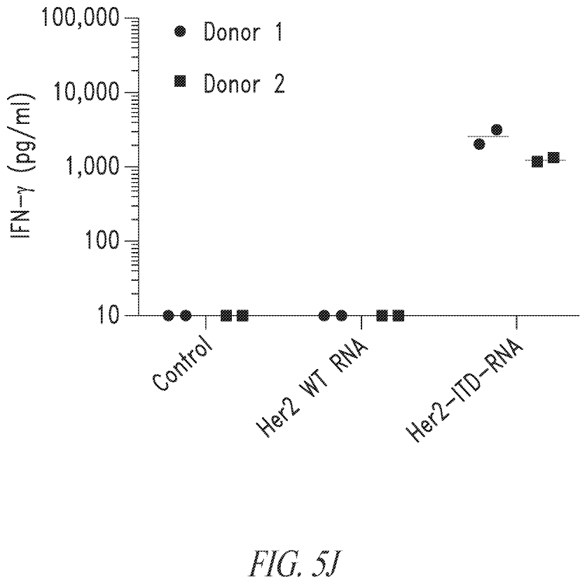

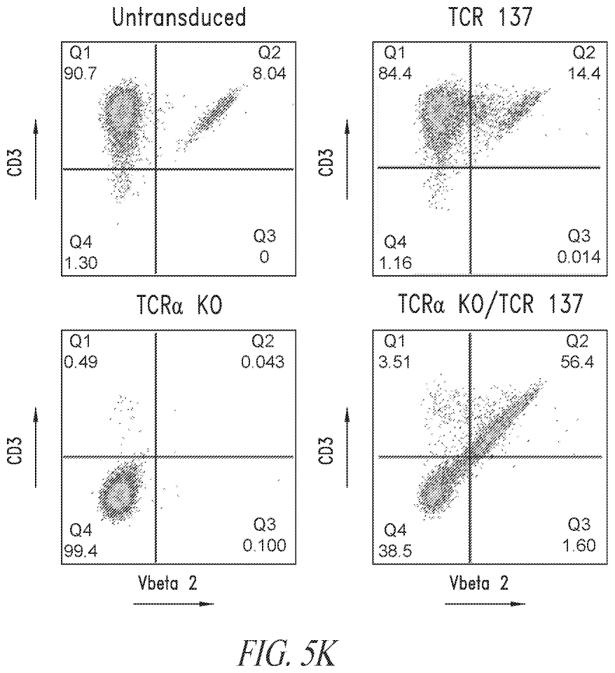

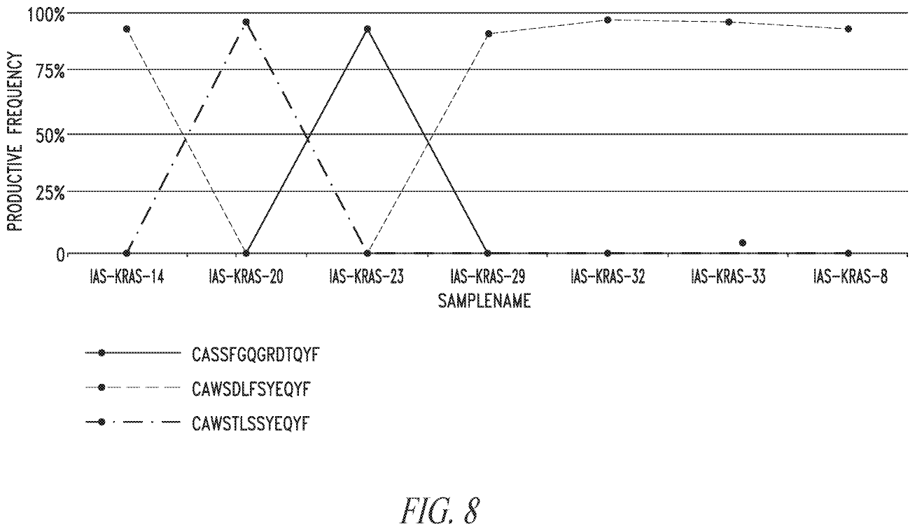

Analysis of multiple T-cell lines by TCRVβ sequencing revealed a single recurrent TCRVβ clonotype present in all ten T-cell lines (FIG. 6B; data for KRAS clonotypes shown in FIG. 8), which was nearly clonal in one T-cell line (#35). This line recognized the mutant Her2-ITD peptide at low peptide concentrations, but not the corresponding wild-type Her2 peptide (FIGS. 5A, 5B), and reactivity was completely blocked by anti-HLA-DQ, but not anti-HLA-DR or anti-class I (FIGS. 5C, 5D). Consistent with the blocking data, the T cells reacted only with Her2-ITD peptide—pulsed B-LCL lines expressing HLA-DQB1*05:01 and 05:02, suggesting HLA restriction by HLA DQB1-05 (FIG. 5G). These T cells also specifically recognized MHC class II+ cells transfected with mutant but not wild-type Her2 sequences targeted to the endosome (FIGS. 5E, 5F). TCRVβ and Vα sequences of the Her2-ITD-specific line were obtained by 5′ RACE. Lentiviral gene transfer of the TCR sequences, following the disruption of the endogenous TCRα by CRISPR-Cas9-mediated gene deletion, conferred specificity to the Her2-ITD peptide and MHC class 11+ cells transfected with the mutant, but not wild-type, Her2 sequences (FIG. 5H-5J). The expression of the transferred TCRs, measured by staining with a Vβ2-specific antibody, was improved by CRISPR-mediated deletion of the endogenous TCRα constant region gene TRAC (FIG. 5K).

TCRVβ deep-sequencing of the initial lung resection sample from patient 1238 identified the Her2-ITD-specific TCRVβ clonotype in 3 of 20179 templates in the tumor resection. Despite five-fold deeper sequencing of the non-adjacent lung tissue from the resection, no Her2-ITD-specific clonotype was observed, showing enrichment of Her2-reactive CD4+ T cells in the tumor (FIG. 5L, p=0.004 for enrichment). The presence of Her2-ITD-specific CD4+ T cells in the blood 2 years after tumor resection is consistent with these cells being part of a persistent memory T-cell response to the tumor.

The present disclosure also provides the following exemplary embodiments:

Embodiment 1. A binding protein comprising:

a T cell receptor (TCR) α-chain variable domain (Vα) comprising a CDR3 amino acid sequence that is at least about 85% identical to the amino acid sequence of SEQ ID NO:2 or SEQ ID NO:12; and

a TCR β-chain variable domain (Vβ) comprising a CDR3 amino acid sequence that is at least about 85% identical to the amino acid sequence of SEQ ID NO:3 or SEQ ID NO:13,

wherein the binding protein is capable of binding to a MTEYKLVVV GAVGVGKSALTIQLIQ (SEQ ID NO:1):human leukocyte antigen (HLA) complex, and/or to a peptide:HLA complex wherein the peptide comprises or consists of 7, 8, 9, 10, 11, 12, 13, 14, 15, 16, 27, 28, 19, 20, 21, 22, 23, or 24 contiguous amino acids of SEQ ID NO:1).

Embodiment 2. The binding protein of embodiment 1, wherein the Vα comprises the CDR3 amino acid sequence of SEQ ID NO:2 and the Vβ comprises the CDR3 amino acid sequence of SEQ ID NO:3.

Embodiment 3. The binding protein of embodiment 1, wherein the Vα comprises the CDR3 amino acid sequence of SEQ ID NO:12 and the Vβ comprises the CDR3 amino acid sequence of SEQ ID NO:13.

Embodiment 4. The binding protein of any one of embodiments 1-3, further comprising:

(i) a CDR1α amino acid sequence according to SEQ ID NO:48 or 54;

(ii) a CDR2α amino acid sequence according to SEQ ID NO:49 or 55;

(iii) a CDR1β amino acid sequence according to SEQ ID NO:51 or 57; and/or

(iv) a CDR2β amino acid sequence according to SEQ ID NO:52 or 58.

Embodiment 5. The binding protein of embodiment 4, comprising CDR1α, CDR2α, CDR3α, CDR1β, CDR2β, and CDR3β amino acid sequences as set forth in SEQ ID NOs:48, 49, 2, 51, 52, and 3, respectively.

Embodiment 6. The binding protein of embodiment 5, comprising CDR1α, CDR2α, CDR3α, CDR1β, CDR2β, and CDR3β amino acid sequences as set forth in SEQ ID NOs:54, 55, 12, 57, 58, and 13, respectively.

Embodiment 7. The binding protein of any one of embodiments 1-6, wherein the HLA comprises DRB1-1101 or DRB1-1104.

Embodiment 8. The binding protein of any one of embodiments 1-7, wherein the Vα comprises or consists of an amino acid sequence that is at least about 85% identical to the amino acid sequence of any one of SEQ ID NOs:6, 16, 66, or 70.

Embodiment 9. The binding protein of any one of embodiments 1-8, wherein the Vβ comprises or consists of an amino acid sequence that that is at least 85% identical to an amino acid sequence of any one of SEQ ID NOs:9,19, 68, or 72.

Embodiment 10. The binding protein of any one of embodiments 1-9, wherein at least three or four of the complementary determining regions (CDRs) of the Vα and/or the Vβ have no change in sequence, and wherein the CDRs that do have sequence changes have only up to two amino acid substitutions, up to a contiguous five amino acid deletion, or a combination thereof.

Embodiment 11. The binding protein of any one of embodiments 1-10, wherein the Vα comprises an amino acid sequence that is at least 85% identical to an amino acid sequence according to TRAV8-3 or TRAV8-1.

Embodiment 12. The binding protein of any one of embodiments 1-11, wherein the Vβ comprises an amino acid sequence that is at least about 85% identical to an amino acid sequence according to TRBV30 or TRBV12-4.

Embodiment 13. The binding protein of any one of embodiments 1-12, further comprising: an amino acid sequence that is at least 85% identical to an amino acid sequence according to TRAJ13 or TRAJ38; and

an amino acid sequence according to a TCR β-chain joining (Jβ) gene segment.

Embodiment 14. The binding protein of embodiment 13, comprising an amino acid sequence that is at least 85% identical to an amino acid sequence according to TRBJ2-4 or TRBJ2-3.

Embodiment 15. The binding protein of any one of embodiments 1-14, wherein the Vα comprises or consists of the amino acid sequence set forth in SEQ ID NO:6 or 66, and the Vβ comprises or consists of the amino acid sequence set forth in SEQ ID NO:9 or 68.

Embodiment 16. The binding protein of any one of embodiments 1-14, wherein the Vα comprises or consists of the amino acid sequence set forth in SEQ ID NO:16 or 70, and the Vβ comprises or consists of the amino acid sequence set forth in SEQ ID NO:19 or 72.

Embodiment 17. The binding protein of any one of embodiments 1-16, further comprising a TCR β chain constant domain (C13), a TCR α chain constant domain (Cα), or both.

Embodiment 18. The binding protein of embodiment 17, wherein:

(i) the Cα has at least about 85% identity to, comprises, or consists of the amino acid sequence set forth in SEQ ID NO:67 or 71; and/or

(ii) the Cβ has at least about 85% identity to, comprises, or consists of the amino acid sequence set forth in SEQ ID NO:69 or 73.

Embodiment 19. The binding protein of any one of embodiments 1-18, wherein the binding protein is capable binding to a (SEQ ID NO:1):HLA complex, and/or to peptide:HLA complex wherein the peptide comprises or consists of about 7, 8, 9, 10, 11, 12, 13, 14, 15, 16, 27, 28, 19, 20, 21, 22, 23, or 24 contiguous amino acids of SEQ ID NO:1, on a cell surface independent or in the absence of CD4.

Embodiment 20. A binding protein comprising:

a T cell receptor (TCR) α-chain variable (Vα) domain comprising a CDR3 amino acid sequence that is at least about 85% identical to the amino acid sequence of SEQ ID NO:23; and

a TCR β-chain variable domain(Vβ) comprising a CDR3 amino acid sequence that is at least about 85% identical to the amino acid sequence of

SEQ ID NO:24, wherein the binding protein is capable of binding to a SPKANKEILDEAYVMAYVMAGVGSPYVSRLLG (SEQ ID NO:22):human leukocyte antigen (HLA) complex and/or to a peptide:HLA complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO:22.

Embodiment 21. The binding protein of embodiment 20, wherein the Vα comprises the CDR3 amino acid sequence of SEQ ID NO:23 and the Vβ comprises the CDR3 amino acid sequence of SEQ ID NO:24.

Embodiment 22. The binding protein of any one of embodiments 20 or 21, further comprising a CDR1α according to SEQ ID NO:60, a CDR2α according to SEQ ID NO:61, a CDR1β according to SEQ ID NO:63, and/or a CDR2β according to SEQ ID NO:64.

Embodiment 23. The binding protein of embodiment 22, comprising CDR1α, CDR2α, CDR3α, CDR1β, CDR2β, and CDR3β amino acid sequences as set forth in SEQ ID NOs:60, 61, 23, 63, 64, and 24, respectively.

Embodiment 24. The binding protein of any one of embodiments 20-23, wherein the HLA comprises DQB1-05:01 or DQB1-05:02.

Embodiment 25. The binding protein of any one of embodiments 20-24, wherein the Vα comprises or consists of an amino acid sequence that is at least about 85% identical to the amino acid sequence of SEQ ID NO:27 or 74.

Embodiment 26. The binding protein of any one of embodiments 20-25, wherein the Vβ comprises or consists of an amino acid sequence that is at least about 85% identical to the amino acid sequence of SEQ ID NO:30 or 76.

Embodiment 27. The binding protein of any one of embodiments 20-26, wherein at least three or four of the complementary determining regions (CDRs) have no change in sequence, and wherein the CDRs that do have sequence changes have only up to two amino acid substitutions, up to a contiguous five amino acid deletion, or a combination thereof.

Embodiment 28. The binding protein of any one of embodiments 20-27, wherein the Vα comprises an amino acid sequence that is at least about 85% identical to an amino acid sequence according to TRAV8-6.

Embodiment 29. The binding protein of any one of embodiments 20-28, wherein the Vβ comprises an amino acid sequence that is at least about 85% identical to an amino acid sequence according to TRBV20.

Embodiment 30. The binding protein of any one of embodiments 20-29, further comprising:

amino acid sequence that is at least about 85% identical to an amino acid sequence according to TRAJ34; and

an amino acid sequence according to a TCR β-chain joining (Jβ) gene segment.

Embodiment 31. The binding protein of embodiment 30, comprising an amino acid sequence that is at least about 85% identical to an amino acid sequence according to TRBJ2-5.

Embodiment 32. The binding protein of any one of embodiments 1-31, wherein the Vα comprises or consists of the amino acid sequence set forth in SEQ ID NO:27 or 74, and the Vβ comprises or consists of the amino acid sequence set forth in SEQ ID NO:30 or 76.

Embodiment 33. The binding protein of any one of embodiments 20-32, further comprising a TCR β chain constant domain (Cβ), a TCR α chain constant domain (Cα), or both.

Embodiment 34. The binding protein of embodiment 33, wherein:

the Cα has at least about 85% identity to, comprises, or consists of the amino acid sequence set forth in SEQ ID NO:75; and/or

(ii) the Cβ has at least about 85% identity to, comprises, or consists of the amino acid sequence set forth in SEQ ID NO:77.

Embodiment 35. The binding protein of any one of embodiments 20-34, wherein the binding protein is capable binding to a (SEQ ID NO:22):HLA complex, and/or to a peptide:HLA complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO:22 on a cell surface independent or in the absence of CD4.

Embodiment 36. The binding protein of any one of embodiments 1-35, wherein the binding protein is a TCR, a chimeric antigen receptor, or an antigen-binding fragment of a TCR.

Embodiment 37. The binding protein of embodiment 36, wherein the TCR, the chimeric antigen receptor, or the antigen-binding fragment of the TCR is chimeric, humanized, or human.

Embodiment 38. The binding protein of embodiment 36 or embodiment 37, wherein the antigen-binding fragment of the TCR comprises a single chain TCR (scTCR).

Embodiment 39. A composition comprising the binding protein of any one of embodiments 1-38 and a pharmaceutically acceptable carrier, diluent, or excipient.

Embodiment 40. A polynucleotide encoding the binding protein of any one of embodiments 1-39.

Embodiment 41. The polynucleotide of embodiment 40, wherein the polynucleotide is codon optimized.

Embodiment 42. The polynucleotide of embodiment 40 or 41, wherein the polynucleotide comprises or consists of a nucleotide sequence having at least 70% identity to the nucleotide sequence set forth in any one of SEQ ID NOs: 4, 5, 7, 8, 10, 14, 15, 17, 18, 20, 25, 26, 28, 29, or 31.

Embodiment 43. The polynucleotide of any one of embodiments 40-42, wherein the encoded binding protein comprises a TCRα chain and a TCRβ chain, wherein the polynucleotide further comprises a polynucleotide encoding a self-cleaving peptide disposed between the α-chain-encoding polynucleotide and the p-chain-encoding polynucleotide.

Embodiment 44. An expression vector, comprising the polynucleotide of any one of embodiments 40-43 operably linked to an expression control sequence.

Embodiment 45. The expression vector of embodiment 44, wherein the expression vector is capable of delivering the polynucleotide to a host cell.

Embodiment 46. The expression vector of embodiment 45, wherein the host cell is a hematopoietic progenitor cell or a human immune system cell.

Embodiment 47. The expression vector of embodiment 46, wherein the immune system cell is a CD4+ T cell, a CD8+ T cell, a CD4− CD8− double negative T cell, a γδ T cell, a natural killer cell, a dendritic cell, or any combination thereof.

Embodiment 48. The expression vector of embodiment 47, wherein the T cell is a naïve T cell, a central memory T cell, an effector memory T cell, or any combination thereof.

Embodiment 49. The expression vector of any one of embodiments 44-48, wherein the expression vector is a viral vector.

Embodiment 50. The expression vector of embodiment 49, wherein the viral vector is a lentiviral vector or a γ-retroviral vector.

Embodiment 51. A recombinant host cell, comprising the polynucleotide of any one of embodiments 40-43 or the expression vector of any one of embodiments 44-50, wherein the recombinant host cell is capable of expressing on its cell surface the encoded binding protein, wherein the polynucleotide is heterologous to the host cell.

Embodiment 52. The recombinant host cell of embodiment 51, wherein the recombinant host cell is a hematopoietic progenitor cell or an immune system cell, optionally a human immune system cell.

Embodiment 53. The recombinant host cell of embodiment 52, wherein the immune system cell is a CD4+ T cell, a CD8+ T cell, a CD4− CD8− double negative T cell, a γδ T cell, a natural killer cell, a dendritic cell, or any combination thereof.

Embodiment 54. The recombinant host cell of embodiment 52 or 53, wherein the immune system cell is a T cell.

Embodiment 55. The recombinant host cell of embodiment 53 or 54, wherein the T cell is a naïve T cell, a central memory T cell, an effector memory T cell, a stem cell memory T cell, or any combination thereof.

Embodiment 56. The recombinant host cell of any one of embodiments 52-55, wherein the binding protein is capable of more efficiently associating with a CD3 protein as compared to an endogenous TCR.

Embodiment 57. The recombinant host cell of any one of embodiments 52-55, wherein the binding protein has a higher surface expression as compared to an endogenous TCR.

Embodiment 58. The recombinant host cell of any one of embodiments 52-57, which is capable of producing IFN-γ when in the presence of a peptide antigen:HLA complex, but produces a lesser amount of, or produces no detectable, IFN-γ when in the presence of a reference peptide: HLA complex,

wherein the peptide antigen is according to SEQ ID NO:1 or 22, or wherein the peptide antigen comprises or consists of about 7, 8, 9, 10, 11, 12, 13, 14, 15, 16, 27, 28, 19, 20, 21, 22, 23, or 24 contiguous amino acids of SEQ ID NO:1 or 22, respectively, and wherein the reference peptide is according to SEQ ID NO:33 or 34, respectively.

Embodiment 59. The recombinant host cell of embodiment 58, which is capable of producing IFN-γ when the peptide antigen is present at a concentration of 10, 1, 0.1, or about 0.01 μg/m L.

Embodiment 60. The recombinant host cell of any one of embodiments 58 or 59, which is capable of producing at least about 1,000, 2,000, 3,000, 4,000, 5,000, 6,000, 7,000, 8,000, 9,000, or 10,000 μg/mL IFN-γ when in the presence of the peptide antigen:HLA complex, wherein the peptide antigen is present at a concentration from 0.01 μg/mL to about 100 μg/mL.

Embodiment 61. The recombinant host cell of any one of embodiments 58-60, which is capable of producing IFNγ in the presence of:

(a) a KRAS G12V peptide:HLA complex; and

(b)(i) an anti-HLA-DQ antibody or (b)(ii) an anti-HLA-DR antibody.

Embodiment 62. The recombinant host cell of any one of embodiments 58-61, which is capable of producing IFNγ in the presence of (i) a KRAS G12V peptide antigen and/or a KRAS G12V peptide-encoding RNA and (ii) a cell that expresses HLA-DRB1-1101 or HLA DRB1-1104 and is capable of presenting a KRAS G12V antigen to the host immune cell.

Embodiment 63. The recombinant host cell of any one of embodiments 58-62, which:

(i) is capable of producing at least about 50 μg/mL IFN-γ when in the presence of the peptide antigen:HLA complex, wherein the peptide antigen is according to SEQ ID NO:22 and is present at about 0.01 μg/mL or about 0.05 μg/mL; and/or

(ii) is capable of producing at least about 100, 500, 1000, 5,000, or 10,000 μg/mL IFN-γ when in the presence of the peptide antigen:HLA complex, wherein the peptide antigen is according to SEQ ID NO:22 (or comprises or consists of about 7, 8, 9, 10, 11, 12, 13, 14, 15, 16, 17, 18, 19, 20, 21, 22, 23, 24, 25, 26, 27, 28, 29, 30, or 31 contiguous amino acids of SEQ ID NO:22) and is present at about 0.02, 0.2, 2, or 20pg/mL.

Embodiment 64. The recombinant host cell of any one of embodiments 58-63, which is capable of producing at least about 10,000 μg/mL IFN-γ when in the presence of a peptide antigen:HLA complex, wherein the peptide antigen is according to SEQ ID NO:22 and is present at at least about 0.01 μg/mL.

Embodiment 65. The recombinant host cell of any one of embodiments is 58-64, which is capable of producing IFN-γ when in the presence a peptide antigen:HLA complex and an anti-HLA-DR antibody and/or an anti-HLA Class I antibody, wherein the peptide antigen is according to SEQ ID NO:22.

Embodiment 66. The recombinant host cell of any one of embodiments 58-65, which is capable of producing IFN-γ when in the presence of (i) a Her2-ITD peptide antigen according to SEQ ID NO:22 and/or a polynucleotide that encodes SEQ ID NO:22 and (ii) a cell line that expresses HLA-DQB1-0501 or HLA-DQB1-0502 and is capable of presenting the Her2-ITD peptide antigen to the host immune cell.

Embodiment 67. The recombinant host cell of any one of embodiments 58-66, which is an immune cell and comprises a chromosomal gene knockout of an endogenous immune cell protein.

Embodiment 68. The recombinant host cell of embodiment 67, comprising a chromosomal gene knocout of a PD-1, a TIM3, a LAG3, a CTLA4, a TIGIT, an HLA component, a TCR component, or any combination thereof.

Embodiment 69. A method of treating a subject in need thereof, the method comprising:

administering an effective amount of a composition comprising the binding protein of any one of embodiments 1-38, or the recombinant host cell of any one of embodiments 58-68 to the subject, wherein the subject has non-small cell lung cancer (NSCLC), colorectal cancer, pancreas cancer, ovarian cancer, breast cancer, biliary tract cancer, an indication wherein a KRAS G12V neoantigen is a therapeutic target, or an indication wherein a Her2-ITD neoantigen is a therapeutic target.

Embodiment 70. The method of embodiment 69, wherein the composition is administered parenterally or intravenously.

Embodiment 71. The method of embodiment 69 or embodiment 70, wherein the method comprises administering a plurality of doses of the composition to the subject.

Embodiment 72. The method of embodiment 71, wherein the plurality of doses are administered at intervals between administrations of about two to about four weeks.

Embodiment 73. The method of any one of embodiments 69-72, wherein the method further comprises administering a cytokine to the subject.

Embodiment 74. The method of embodiment 73, wherein the cytokine comprises IL-2, IL-15, or IL-21.

Embodiment 75. The method of any one of embodiments 69-74, wherein the subject is further receiving immunosuppressive therapy.

Embodiment 76. The method of any one of embodiments 69-75, further comprising administering an immune suppression agent inhibitor, optionally a PD-1 inhibitor, to the subject.

Embodiment 77. The method of embodiment 76, wherein the PD-1 inhibitor comprises nivolumab (OPDIVO®); pembrolizumab (KEYTRUDA®); ipilimumab+nivolumab (YERVOY®+OPDIVO®); cemiplimab; IBI-308; nivolumab+relatlimab; BCD-100; camrelizumab; JS-001; spartalizumab; tislelizumab; AGEN-2034; BGBA-333+tislelizumab; CBT-501; dostarlimab; durvalumab+MEDI-0680; JNJ-3283; pazopanib hydrochloride+pembrolizumab; pidilizumab; REGN-1979+cemiplimab; ABBV-181; ADUS-100+spartalizumab; AK-104; AK-105; AMP-224; BAT-1306; BI-754091; CC-90006; cemiplimab+REGN-3767; CS-1003; GLS-010; LZM-009; MEDI-5752; MGD-013; PF-06801591; Sym-021; tislelizumab+pamiparib; XmAb-20717; AK-112; ALPN-202; AM-0001; an antibody to antagonize PD-1 for Alzheimer's disease; BH-2922; BH-2941; BH-2950; BH-2954; a biologic to antagonize CTLA-4 and PD-1 for solid tumor; a bispecific monoclonal antibody to target PD-1 and LAG-3 for oncology; BLSM-101; CB-201; CB-213; CBT-103; CBT-107; a cellular immunotherapy+PD-1 inhibitor; CX-188; HAB-21; HEISCOIII-003; IKT-202; JTX-4014; MCLA-134; MD-402; mDX-400; MGD-019; a monoclonal antibody to antagonize PDCD1 for oncology; a monoclonal antibody to antagonize PD-1 for oncology; an oncolytic virus to inhibit PD-1 for oncology; OT-2; PD-1 antagonist+ropeginterferon alfa-2b; PEGMP-7; PRS-332; RXI-762; STIA-1110; TSR-075; a vaccine to target HER2 and PD-1 for oncology; a vaccine to target PD-1 for oncology and autoimmune disorders; XmAb-23104; an antisense oligonucleotide to inhibit PD-1 for oncology; AT-16201; a bispecific monoclonal antibody to inhibit PD-1 for oncology; IMM-1802; monoclonal antibodies to antagonize PD-1 and CTLA-4 for solid tumor and hematological tumor; nivolumab biosimilar; a recombinant protein to agonize CD278 and CD28 and antagonize PD-1 for oncology; a recombinant protein to agonize PD-1 for autoimmune disorders and inflammatory disorders; SNA-01; SSI-361; YBL-006; AK-103; JY-034; AUR-012; BGB-108; drug to inhibit PD-1, Gal-9, and TIM-3 for solid tumor; ENUM-244C8; ENUM-388D4; MEDI-0680; monoclonal antibodies to antagonize PD-1 for metastatic melanoma and metastatic lung cancer; a monoclonal antibody to inhibit PD-1 for oncology; monoclonal antibodies to target CTLA-4 and PD-1 for oncology; a monoclonal antibody to antagonize PD-1 for NSCLC; monoclonal antibodies to inhibit PD-1 and TIM-3 for oncology; a monoclonal antibody to inhibit PD-1 for oncology; a recombinant protein to inhibit PD-1 and VEGF-A for hematological malignancies and solid tumor; a small molecule to antagonize PD-1 for oncology; Sym-016; inebilizumab+MEDI-0680; a vaccine to target PDL-1 and IDO for metastatic melanoma; an anti-PD-1 monoclonal antibody+a cellular immunotherapy for glioblastoma; an antibody to antagonize PD-1 for oncology; monoclonal antibodies to inhibit PD-1/PD-L1 for hematological malignancies and bacterial infections; a monoclonal antibody to inhibit PD-1 for HIV; or a small molecule to inhibit PD-1 for solid tumor.

Embodiment 78. The method of any one of embodiments 69-77, wherein the composition comprises a recombinant CD4+ T cell, a recombinant CD8+ T cell, or both.

Embodiment 79. The method of any one of embodiments 69-78, wherein the recombinant host cell is allogeneic, autologous, or syngeneic.

Embodiment 80. The binding protein of any one of embodiments 1-38, the composition of embodiment 39, the polynucleotide of any one of embodiments 40-43, the expression vector of any one of embodiments 44-50, or the recombinant host cell of any one of embodiments 51-68 for use in the treatment of non-small cell lung cancer (NSCLC), colorectal cancer, pancreas cancer, ovarian cancer, breast cancer, biliary tract cancer, an indication wherein a KRAS G12V neoantigen is a therapeutic target, or an indication wherein a Her2-ITD neoantigen is a therapeutic target.

Embodiment 81. The recombinant host cell of any one of embodiments 51-68 for use in adoptive immunotherapy of non-small cell lung cancer (NSCLC), colorectal cancer, pancreas cancer, ovarian cancer, breast cancer, biliary tract cancer, an indication wherein a KRAS G12V neoantigen is a therapeutic target, or an indication wherein a Her2-ITD neoantigen is a therapeutic target.

Embodiment 82. The binding protein of any one of embodiments 1-38, the composition of embodiment 39, the polynucleotide of any one of embodiments 40-43, the expression vector of any one of embodiments 44-50, or the recombinant host cell of any one of embodiments 51-68 for use in the manufacture of a medicament for the treatment of non-small cell lung cancer (NSCLC), colorectal cancer, pancreas cancer, ovarian cancer, breast cancer, biliary tract cancer, an indication wherein a KRAS G12V neoantigen is a therapeutic target.

Embodiment 83. An immunogenic composition comprising:

(i) a peptide having an amino acid sequence that is at least 80% identical to MTE YKL VVV GAV GVG KSA LTI QLI Q (SEQ ID NO:1) or SPK ANK EIL DEA YVM AYV MAG VGS PYV SRL LG (SEQ ID NO:22); and

(ii) a non-naturally occurring pharmaceutically acceptable carrier.

Embodiment 84. The immunogenic composition of embodiment 83, wherein the non-naturally occurring pharmaceutically acceptable carrier comprises a cream, emulsion, gel, liposome, nanoparticle, or ointment.

Embodiment 85. An immunogenic composition comprising:

(i) a peptide having an amino acid sequence that is at least 80% identical to MTE YKL WV GAV GVG KSA LTI QLI Q (SEQ ID NO:1) or SPK ANK EIL DEA YVM AYV MAG VGS PYV SRL LG (SEQ ID NO:22); and

(ii) an immuno-effective amount of an adjuvant.

Embodiment 86. The immunogenic composition of embodiment 84, wherein the adjuvant comprises poly-ICLC, CpG, GM-CSF, or alum.

Embodiment 87. A method of treating a subject in need thereof, or of inducing an immune response in a subject, the method comprising administering the immunogenic composition of any one of embodiments 83-86 to the subject,

wherein the subject has, or is suspected of having, non-small cell lung cancer (NSCLC), colorectal cancer, pancreas cancer, ovarian cancer, breast cancer, biliary tract cancer, an indication wherein a KRAS G12V neoantigen is a therapeutic target, or an indication wherein a Her2-ITD neoantigen is a therapeutic target.

Embodiment 88. The method of embodiment 87, wherein the immunogenic composition is administered two or more times to the subject.

Embodiment 89. The method of embodiment 87 or embodiment 88, further comprising administering an adoptive cell therapy to the subject.

Embodiment 90. The method of any one of embodiments 86-88, further comprising administering at least one of an adjuvant or a checkpoint inhibitor to the subject, wherein the adjuvant or the checkpoint inhibitor optionally comprises at least one of IL-2, a PD-1 inhibitor, a PD-L1 inhibitor, or a CTLA-4 inhibitor.

Embodiment 91. An isolated peptide capable of eliciting an antigen-specific T-cell response to KRAS G12V, comprising a polypeptide of no more than 25, 24, 23, 22, 21, 20, 19, 18, 17, 16, 15, 14, 13, 12, 11, 10, 9, 8, or 7 amino acids wherein the polypeptide comprises a sequence of at least 7, 8, 9, 10, 11, 12, 13, 14, 15, 16, 17, 18, 19, 20, 21, 22, 23, 24, or 25 contiguous amino acids from the KRAS G12V amino acid sequence set forth in SEQ ID NO:1.

Embodiment 92. An isolated peptide capable of eliciting an antigen-specific T-cell response to Her2-ITD, comprising a polypeptide of no more than 32, 31, 30, 29, 28, 27, 26, 25, 24, 23, 22, 21, 20, 19, 18, 17, 16, 15, 14, 13, 12, 11, 10, 9, 8, or 7 amino acids wherein the polypeptide comprises a sequence of at least 7, 8, 9, 10, 11, 12, 13, 14, 15, 16, 17, 18, 19, 20, 21, 22, 23, 24, 25, 26, 27, 28, 29, 30, 31, or 32 contiguous amino acids from the Her2-ITD amino acid sequence set forth in SEQ ID NO:22.

Embodiment 93. A method for preparing antigen-pulsed antigen-presenting cells, the method comprising:

contacting in vitro, under conditions and for a time sufficient for antigen processing and presentation by antigen-presenting cells to take place, (i) a population of antigen-presenting cells, and (ii) a polynucleotide of any one of embodiments 40-43 or an expression vector of any one of embodiments 44-50, thereby obtaining antigen-pulsed antigen-presenting cells capable of eliciting an antigen-specific T-cell response to KRAS G12V or Her2-ITD.

Embodiment 94. The method of embodiment 93, further comprising contacting the antigen-pulsed antigen-presenting cells with one or a plurality of immunocompatible T cells under conditions and for a time sufficient to generate KRAS G12V-specific T cells or Her2-ITD-specific T cells.

Embodiment 95. A method comprising expanding in vitro the KRAS G12V-specific T cells or the Her2-ITD-specific T cells of embodiment 93 to thereby obtain one or more clones of the KRAS G12V-specific T cells or the Her2-ITD-specific T cells, respectively, and determining a T cell receptor polypeptide encoding nucleic acid sequence for one or more of the one or more clones.

Embodiment 96. The method of embodiment 95, further comprising transfecting or transducing a T cell population in vitro with a polynucleotide having the T-cell receptor polypeptide-encoding nucleic acid sequence so-determined, thereby obtaining a population of engineered KRAS G12V-specific T cells or engineered Her2-ITD-specific T cells in an amount effective to adoptively transfer an antigen-specific T-cell response.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/721,439, filed on Aug. 22, 2018, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A binding protein comprising:
a T cell receptor (TCR) α-chain variable domain (Vα) comprising a CDR3 amino acid sequence that is at least about 85% identical to the amino acid sequence of SEQ ID NO.:2 or SEQ ID NO.:12; and
a TCR β-chain variable domain (Vβ) comprising a CDR3 amino acid sequence that is at least about 85% identical to the amino acid sequence of SEQ ID NO.:3 or SEQ ID NO.:13,
wherein the binding protein is capable of binding to a MTEYKLVVV GAVGVGKSALTIQLIQ (SEQ ID NO.:1): human leukocyte antigen (HLA) complex, and/or to a peptide:HLA complex wherein the peptide comprises or consists of 7, 8, 9, 10, 11, 12, 13, 14, 15, 16, 27, 28, 19, 20, 21, 22, 23, or 24 contiguous amino acids of SEQ ID NO.:1).

2. The binding protein of claim 1, wherein the Vα comprises the CDR3 amino acid sequence of SEQ ID NO.:2 and the Vβ comprises the CDR3 amino acid sequence of SEQ ID NO.:3, or wherein the Vα comprises the CDR3 amino acid sequence of SEQ ID NO.:12 and the Vβ comprises the CDR3 amino acid sequence of SEQ ID NO.:13.

3. (canceled)

4. The binding protein of claim 1, further comprising:
(i) a CDR1α amino acid sequence according to SEQ ID NO.:48 or 54;
(ii) a CDR2α amino acid sequence according to SEQ ID NO.:49 or 55;
(iii) a CDR1β amino acid sequence according to SEQ ID NQ:51 or 57; and/or
(iv) a CDR2β amino acid sequence according to SEQ ID NO.:52 or 58.

5. The binding protein of claim 4, comprising CDR1α, CDR2α, CDR3α, CDR1β, CDR2β, and CDR3β amino acid sequences as set forth in:
(i) SEQ ID NOs:48, 49, 2, 51, 52, and 3, respectively; or
(ii) SEQ ID NOs.:54, 55, 12, 57, 58, and 13, respectively.

6. (canceled)

7. The binding protein of claim 1, wherein the HLA comprises DRB1-1101 or DRB1-1104.

8. The binding protein of claim 1, wherein:
(i) the Vα comprises or consists of an amino acid sequence that is at least about 85% identical to the amino acid sequence of SEQ ID NO.:66 or SEQ ID NO.:70; and/or
(ii) the Vβ comprises or consists of an amino acid sequence that that is at least 85% identical to the amino acid sequence of SEQ ID NO.:68 or SEQ ID NO.:72.

9. (canceled)

10. The binding protein of claim 8, wherein at least three or four of the complementary determining regions (CDRs) of the Vα and/or the Vβ have no change in sequence, and wherein the CDRs that do have sequence changes have only up to two amino acid substitutions, up to a contiguous five amino acid deletion, or a combination thereof.

11. The binding protein of claim 1, wherein:
(i) the Vα comprises an amino acid sequence that is at least 85% identical to an amino acid sequence according to TRAV8-3 or TRAV8-1; and/or
(ii) the Vβ comprises an amino acid sequence that is at least about 85% identical to an amino acid sequence according to TRBV30 or TRBV12-4; and/or
(iii) the binding protein further comprises an amino acid sequence that is at least 85% identical to an amino acid sequence according to TRAJ13 or TRAJ38, and an amino acid sequence according to a TCR β-chain joining (Jβ) gene segment; or
(iv) the binding protein further comprises an amino acid sequence that is at least 85% identical to an amino acid sequence according to TRAJ13 or TRAJ38, and an amino acid sequence that is at least 85% identical to an amino acid sequence according to TRBJ2-4 or TRBJ2-3.

12.-14. (canceled)

15. The binding protein of claim 1, wherein:
(i) the Vα comprises or consists of the amino acid sequence set forth in SEQ ID NO.: 66, and the Vβ comprises or consists of the amino acid sequence set forth in SEQ ID NO.: 68; or
(ii) the Vα comprises or consists of the amino acid sequence set forth in SEQ ID NO.: 70, and the Vβ comprises or consists of the amino acid sequence set forth in SEQ ID NO.:72.

16. The binding protein of claim 1, further comprising a TCR β chain constant domain (Cβ), a TCR α chain constant domain (Cα), or both.

17. The binding protein of claim 16, comprising:
(i) a TCR β chain that comprises of the amino acid sequence set forth in SEQ ID NO.:6 and a TCR α that comprises or consists of the amino acid sequence set forth in SEQ ID NO.:9; or
(ii) a TCR β chain that comprises of the amino acid sequence set forth in SEQ ID NO.:16 and a TCR α that comprises or consists of the amino acid sequence set forth in SEQ ID NO.:19.

18. The binding protein of claim 16, wherein:
(i) the Cα has at least about 85% identity to, comprises, or consists of the amino acid sequence set forth in SEQ ID NO.:67 or 71; and/or
(ii) the Cβ has at least about 85% identity to, comprises, or consists of the amino acid sequence set forth in SEQ ID NO.:69 or 73.

19. The binding protein of claim 1, wherein the binding protein is capable binding to a (SEQ ID NO.:1):HLA complex, and/or to peptide:HLA complex wherein the peptide comprises or consists of about 7, 8, 9, 10, 11, 12, 13, 14, 15, 16, 27, 28, 19, 20, 21, 22, 23, or 24 contiguous amino acids of SEQ ID NO.:1, on a cell surface independent or in the absence of CD4.

20. A binding protein comprising:
a T cell receptor (TCR) α-chain variable (Vα) domain comprising a CDR3 amino acid sequence that is at least about 85% identical to the amino acid sequence of SEQ ID NO.:23; and
a TCR β-chain variable domain (Vβ) comprising a CDR3 amino acid sequence that is at least about 85% identical to the amino acid sequence of SEQ ID NO.:24,
wherein the binding protein is capable of binding to a SPKANKEILDEAYVMAYVMAGVGSPYVSRLLG (SEQ ID NO.:22):human leukocyte antigen (HLA) complex and/or to a peptide:HLA complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22.

21. The binding protein of claim 20, wherein the Vα comprises the CDR3 amino acid sequence of SEQ ID NO.:23 and the Vβ comprises the CDR3 amino acid sequence of SEQ ID NO.:24.

22. The binding protein of claim 21, further comprising a CDR1α according to SEQ ID NO.:60, a CDR2α-according to SEQ ID NO.:61, a CDR1β according to SEQ ID NO.:63, and/or a CDR2β according to SEQ ID NO.:64.

23. The binding protein of claim 21, comprising CDR1α, CDR2α, CDR3α, CDR1β, CDR2β, and CDR3β amino acid sequences as set forth in SEQ ID NOs.:60, 61, 23, 63, 64, and 24, respectively.

24. The binding protein of claim 20, wherein the HLA comprises DQB1-05:01 or DQB1-05:02.

25. The binding protein of claim 20, wherein:
(i) the Vα comprises or consists of an amino acid sequence that is at least about 85% identical to the amino acid sequence of SEQ ID NO.:74; and/or
(ii) the Vβ comprises or consists of an amino acid sequence that is at least about 85% identical to the amino acid sequence of SEQ ID NO.:30 or 76.

26. (canceled)

27. The binding protein of claim 25, wherein at least three or four of the complementary determining regions (CDRs) have no change in sequence, and wherein the CDRs that do have sequence changes have only up to two amino acid substitutions, up to a contiguous five amino acid deletion, or a combination thereof.

28. The binding protein of claim 20, wherein:
(i) the Vα comprises an amino acid sequence that is at least about 85% identical to an amino acid sequence according to TRAV8-6; and/or
(ii) the VP comprises an amino acid sequence that is at least about 85% identical to an amino acid sequence according to TRBV20; and/or
(iii) the binding protein further comprises an amino acid sequence that is at least 85% identical to an amino acid sequence according to TRAJ34, and an amino acid sequence according to a TCR β-chain joining (Jβ) gene segment; or
(iv) the binding protein further comprises an amino acid sequence that is at least 85% identical to an amino acid sequence according to TRAJ34, and an amino acid sequence that is at least 85% identical to an amino acid sequence according to TRBJ2-5.

29.-31. (canceled)

32. The binding protein of claim 20, wherein the Vα comprises or consists of the amino acid sequence set forth in SEQ ID NO.: 74, and the Vβ comprises or consists of the amino acid sequence set forth in SEQ ID NO.: 76.

33. The binding protein of claim 20, further comprising a TCR β chain constant domain (Cβ), a TCR α chain constant domain (Cα), or both.

34. The binding protein of claim 33, wherein:
(i) the Cα has at least about 85% identity to, comprises, or consists of the amino acid sequence set forth in SEQ ID NO.:75; and/or
(ii) the Cβ has at least about 85% identity to, comprises, or consists of the amino acid sequence set forth in SEQ ID NO.:77.

35. The binding protein of claim 20, wherein the binding protein is capable binding to a (SEQ ID NO.:22):HLA complex, and/or to a peptide:HLA complex wherein the peptide comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22 on a cell surface independent or in the absence of CD4.

36. The binding protein of claim 1, wherein the binding protein is a TCR, a chimeric antigen receptor, or an antigen-binding fragment of a TCR.

37. The binding protein of claim 20, wherein the binding protein is a TCR, a chimeric antigen receptor, or an antigen-binding fragment of a TCR.

38. The binding protein of claim 33, comprising:
(i) a TCR β chain that comprises or consists of the amino acid sequence set forth in SEQ ID NO.:30; and
(ii) a TCR α chain that comprises or consists of the amino acid sequence set forth in SEQ ID NO.:27.

39. A composition comprising the binding protein of claim 1 and a pharmaceutically acceptable carrier, diluent, or excipient.

40. A polynucleotide encoding the binding protein of claim 1.

41. A polynucleotide encoding the binding protein of claim 20.

42. A composition comprising the binding protein of claim 20 and a pharmaceutically acceptable carrier, diluent, or excipient.

43. (canceled)

44. An expression vector, comprising the polynucleotide of claim 40 operably linked to an expression control sequence.

45. An expression vector, comprising the polynucleotide of claim 41 operably linked to an expression control sequence.

46.-50. (canceled)

51. A recombinant host cell, comprising the polynucleotide of claim 40, wherein the recombinant host cell is capable of expressing on its cell surface the encoded binding protein, wherein the polynucleotide is heterologous to the host cell.

52. The recombinant host cell of claim 51, wherein the recombinant host cell:
(i) is a hematopoietic progenitor cell or an immune system cell;
(ii) is a human immune system cell;
(iii) is an immune system cell, wherein the immune system cell is a CD4+ T cell, a CD8+ T cell, a CD4− CD8− double negative T cell, a γδ T cell, a natural killer cell, a dendritic cell, or any combination thereof;
(iv) is an immune system cell, wherein the immune system cell is a T cell; or
(v) is an immune system cell, wherein the immune system cell is a naïve T cell, a central memory T cell, an effector memory T cell, a stem cell memory T cell, or any combination thereof.

53. A recombinant host cell, comprising the polynucleotide of claim 41, wherein the recombinant host cell is capable of expressing on its cell surface the encoded binding protein, wherein the polynucleotide is heterologous to the host cell.

54. The recombinant host cell of claim 53, wherein the recombinant host cell:
(i) is a hematopoietic progenitor cell or an immune system cell;
(ii) is a human immune system cell;
(iii) is an immune system cell, wherein the immune system cell is a CD4+ T cell, a CD8+ T cell, a CD4− CD8− double negative T cell, a γδ T cell, a natural killer cell, a dendritic cell, or any combination thereof;
(iv) is an immune system cell, wherein the immune system cell is a T cell; or
(v) is an immune system cell, wherein the immune system cell is a naïve T cell, a central memory T cell, an effector memory T cell, a stem cell memory T cell, or any combination thereof.

55.-56. (canceled)

57. The recombinant host cell of claim 52:
(i) which is capable of producing IFN-γ when in the presence of a peptide antigen:HLA complex, but produces a lesser amount of, or produces no detectable, IFN-γ when in the presence of a reference peptide:HLA complex, wherein the peptide antigen is according to SEQ ID NO.:1, or wherein the peptide antigen comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:1, respectively, and wherein the reference peptide is according to SEQ ID NO.:33;
(ii) which is capable of producing IFN-γ when the peptide antigen is present at a concentration of 10, 1, 0.1, or about 0.01 μg/mL; and/or
(iii) which is capable of producing at least about 1,000, 2,000, 3,000, 4,000, 5,000, 6,000, 7,000, 8,000, 9,000, or 10,000 μg/mL IFN-γ when in the presence of the peptide antigen:HLA complex, wherein the peptide antigen is present at a concentration from 0.01 μg/mL to about 100 μg/mL; and/or
(iv) which is capable of producing IFNγ in the presence of:
(a) a KRAS G12V peptide:HLA complex; and
(b)(i) an anti-HLA-DQ antibody or (b)(ii) an anti-HLA-DR antibody;
(v) which is capable of producing IFNγ in the presence of (a) a KRAS G12V peptide antigen and/or a KRAS G12V peptide-encoding RNA and (b) a cell that expresses HLA-DRB1-1101 or HLA DRB1-1104 and is capable of presenting a KRAS G12V antigen to the host immune cell; and/or
(vi) wherein the binding protein has a higher surface expression as compared to an endogenous TCR; and/or
(vii) wherein the binding protein is capable of more efficiently associating with a CD3 protein as compared to an endogenous TCR.

58. The recombinant host cell of claim 54:
(i) which is capable of producing IFN-γ when in the presence of a peptide antigen:HLA complex, but produces a lesser amount of, or produces no detectable, IFN-γ when in the presence of a reference peptide:HLA complex, wherein the peptide antigen is according to SEQ ID NO.:22, or wherein the peptide antigen comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22, and wherein the reference peptide is according to SEQ ID NO.:34; and/or
(ii) which is capable of producing IFN-γ when the peptide antigen is present at a concentration of 10, 1, 0.1, or about 0.01 μm/mL; and/or
(iii) which is capable of producing at least about 1,000, 2,000, 3,000, 4,000, 5,000, 6,000, 7,000, 8,000, 9,000, or 10,000 μg/mL IFN-γ when in the presence of the peptide antigen:HLA complex, wherein the peptide antigen is present at a concentration from 0.01 μg/mL to about 100 μg/mL, and/or
(iv) which is capable of producing:
(a) at least about 50 μg/mL IFN-γ when in the presence of the peptide antigen:HLA complex, wherein the peptide antigen is according to SEQ ID NO.:22 or comprises or consists of about 7, 8, 9, 10, 11, 12, 13, 14, 15, 16, 27, 28, 19, 20, 21, 22, 23, 24, 25, 26, 27, 28, 29, 30, or 31 continuous amino acids of SEQ ID NO.:22 and is present at about 0.01 μg/mL or about 0.05 μg/mL, and/or
(b) at least about 100, 500, 1000, 5,000, or 10,000 μg/mL IFN-γ when in the presence of the peptide antigen:HLA complex, wherein the peptide antigen is according to SEQ ID NO.:22 or comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22 and is present at about 0.02, 0.2, 2, or 20 μg/mL;
(v) which is capable of producing at least about 10,000 μg/mL IFN-γ when in the presence of a peptide antigen:HLA complex, wherein the peptide antigen is according to SEQ ID NO.:22 or comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22 and is present at at least about 0.01 μm/mL; and/or
(vi) which is capable of producing IFN-γ when in the presence a peptide antigen:HLA complex and an anti-HLA-DR antibody and/or an anti-HLA Class I antibody, wherein the peptide antigen is according to SEQ ID NO.:22 or comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22; and/or
(vii) which is capable of producing IFN-γ when in the presence of (a) a Her2-ITD peptide antigen according to SEQ ID NO.:22 or comprising or consisting of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22 and/or a polynucleotide that encodes SEQ ID NO.:22 or a peptide that comprises or consists of about 7, 8, 9, 10, 11, 12, 13, 14, 15, 16, 27, 28, 19, 20, 21, 22, 23, 24, 25, 26, 27, 28, 29, 30, or 31 contiguous amino acids of SEQ ID NO.:22 and (b) a cell line that expresses HLA-DQB1-0501 or HLA-DQB1-0502 and is capable of presenting the Her2-ITD peptide antigen to the host immune cell; and/or
(viii) wherein the binding protein has a higher surface expression as compared to an endogenous TCR; and/or
(ix) wherein the binding protein is capable of more efficiently associating with a CD3 protein as compared to an endogenous TCR.

59.-66. (canceled)

67. The recombinant host cell of claim 51, which is an immune cell and comprises a chromosomal gene knockout of an endogenous immune cell protein.

68. The recombinant host cell of claim 53, which is an immune cell and comprises a chromosomal gene knockout of an endogenous immune cell protein.

69. A method of treating a subject in need thereof, the method comprising:
administering an effective amount of a composition comprising the recombinant host cell of claim 51 to the subject, wherein the subject has non-small cell lung cancer (NSCLC), colorectal cancer, pancreas cancer, ovarian cancer, breast cancer, biliary tract cancer, or an indication wherein a KRAS G12V neoantigen is a therapeutic target.

70. A method of treating a subject in need thereof, the method comprising:
administering an effective amount of a composition comprising the recombinant host cell of claim 53 to the subject, wherein the subject has non-small cell lung cancer (NSCLC), colorectal cancer, pancreas cancer, ovarian cancer, breast cancer, biliary tract cancer, or an indication wherein a Her2-ITD neoantigen is a therapeutic target.

71.-96. (canceled)
